(19) United States
(12) Patent Application Publication
(10) Pub. No.: US 2024/0156808 A1
(43) Pub. Date: May 16, 2024

(54) Compositions and methods to improve the therapeutic benefit of suboptimally chemical compounds and biological therapies including substituted camptothecins such as irinotecan and topotecan for the treatment of benign and neoplastic hyperproliferative disease conditions, infections, inflammatory and immunological diseases

(71) Applicant: Edison Oncology, Menlo Park, CA (US)

(72) Inventor: DENNIS BROWN

(21) Appl. No.: 18/278,474

(22) PCT Filed: Feb. 22, 2022
(86) PCT No.: PCT/US22/17308

§ 371 Date: Aug. 23, 2023

Related U.S. Application Data
(60) Provisional application No. 63/152,782, filed on Feb. 23, 2021.

(30) Foreign Application Priority Data
Feb. 23, 2021 (US) 63152782

(51) Int. Cl. A61K 31/4745; A61K 45/06

(52) U.S. Cl. CPC A61K 31/4745; A61K 45/06

(57) ABSTRACT

The present invention is directed to improved methods, formulations, and compositions employing substituted camptothecins such as, but not limited to, irinotecan and topotecan as well as analogs, derivatives, and prodrugs thereof. These methods, formulations, and compositions can be used to treat malignancies and other diseases and conditions including, but not limited to, non-malignant proliferative disorders, infections, inflammatory, and immunological diseases.

### CROSS-REFERENCE TO RELATED APPLICATION

This application claims the benefit of U.S. Provisional Patent Application Ser. No. 63/152,782 by Dennis Brown, entitled “Compositions and Methods to Improve the Therapeutic Benefit of Suboptimally Administered Chemical Compounds and Biologic Therapies Including Substituted Camptothecins Such as Irinotecan and Topotecan for the Treatment of Benign and Neoplastic Hyperproliferative Disease Conditions, Infections, Inflammatory and Immunological Diseases,” filed on Feb. 23, 2001, the contents of which are incorporated herein in their entirety by this reference.

### FIELD OF THE INVENTION

This invention is directed to compositions and methods employing topotecan, irinotecan, or derivatives or analogs of these agents or related topoisomerase inhibitors for treatment of benign and neoplastic hyperproliferative diseases, infections, inflammatory, and immunological diseases.

### BACKGROUND OF THE INVENTION

The search for and identification of cures for many life-threatening diseases that plague humans still remains an empirical and sometimes serendipitous process. While many advances have been made from basic scientific research to improvements in practical patient management, there still remains tremendous frustration in the rational and successful discovery of useful therapies particularly for life-threatening diseases such as cancer, immune-mediated diseases, inflammatory conditions, infection, as well as other diseases and conditions.

Since the “War on Cancer” began in the early 1970's by the United States National Cancer Institute (NCI) of the National Institutes of Health (NIH), a wide variety of strategies and programs have been created and implemented to prevent, diagnose, treat and cure cancer and other life-threatening disease conditions. One of the oldest and arguably most successful programs has been the synthesis and screening of small chemical entities (<1500 MW) for biological activity against cancer. These programs were organized to improve and streamline the progression of discovery and development events from chemical synthesis and molecular biology and biological screening to preclinical studies for the logical progression into human clinical trials with the hope of finding cures for the many types of life-threatening diseases including cancer. The synthesis and screening of hundreds of thousands of chemical compounds from academic and industrial sources, in addition to the screening of natural products and extracts from prokaryotes, invertebrate animals, plants collections, and products or extracts from other sources from all over the world as well as novel products exploited by molecular and synthetic biology methodologies has been and continues to be a major approach for the identification of novel lead structures as potential new and useful medicines. This is in addition to other programs including biotherapeutics designed to stimulate the human immune system with adaptive (e.g. NK cells) and adoptive immune cell transfers (e.g., CAR-T), vaccines, therapeutic antibodies, drug-antibody conjugates, cytokines, lymphokines, cytokine peptides, immune check point inhibitors (PD1/PD-L1), inhibitors of tumor blood vessel development (angiogenesis) or gene and antisense therapies to alter the genetic make-up of cancer cells or alter the functioning of the immune system in order to stimulate it to attack non-self antigens such as those associated with tumors or infectious agents or to repress to treat diseases or conditions characterized by an autoimmune response.

The work supported by the NCI, other governmental agencies both domestic and foreign in academic or industrial research and development laboratories has resulted in an extraordinary body of biological, genomic, pharmacologic, biochemical, chemical and clinical information. In addition, large chemical and biological libraries have been created, as well as highly characterized in silico, in vitro, and in vivo biological screening systems that have been successfully used. However, from the tens of billions of dollars spent over the past fifty years supporting these programs both preclinically and clinically, only a limited number of therapeutics have been identified or discovered that have resulted in the successful development of useful pharmaceutical products. Nevertheless, the biological systems both in vitro and in vivo and the “decision trees” used to warrant further preclinical studies leading to Phase I-III clinical trials have been validated. These drug screening programs, biological models, clinical trial protocols, and other research tools remain critical for the discovery and development of any new therapeutic agent.

Unfortunately, many of the compounds that have successfully met the preclinical testing and federal regulatory requirements for clinical evaluation were either unsuccessful or disappointing in human clinical trials. Many compounds were found to have untoward or idiosyncratic side effects that were discovered during human clinical Phase I dose-escalation studies used to determine the maximum tolerated dose (MTD) and side-effect profile. In some cases, these toxicities or the magnitude of their toxicity were not identified or predicted in preclinical toxicology studies. In other cases, therapeutic agents where in vitro and in vivo studies suggested a potentially unique activity against a particular tumor type, molecular target or biological pathway were not successful in human Phase II clinical trials where specific examination of particular disease indications/types were evaluated in government sanctioned (e.g., United States FDA), IRB approved clinical trials. In addition, there are those cases where potential new agents were evaluated in randomized Phase III clinical trials where a significant clinical benefit could not be demonstrated; such cases have also been the cause of great frustration and disappointment. Finally, a number of compounds have reached regulatory approved commercialization but their ultimate clinical utility has been limited by poor efficacy as monotherapy (e.g., <25% response rates) and for untoward Grade III or IV dose-limiting side effects (e.g., myelosuppression, cardiotoxicity, gastrointestinal toxicities, cytokine storm effects, or other significant dose-limiting side effects) not clearly identified through regulatory clinical trials.

In many cases, after the great time and expense of developing and moving an investigational compound into human clinical trials and where clinical failure has occurred, the tendency has been to return to the laboratory to create a better analog, look for agents with different structures but potentially related mechanisms of action, or attempt other modifications to improve the therapeutic efficacy or reduce the occurrence or severity of side effects. In some cases, efforts have been made to try additional Phase I or II clinical trials in an attempt to make some improvement with the side-effect profile or the therapeutic effect in selected patients or for other disease indications. In many of those cases, the results did not realize a significant enough improvement to warrant further clinical development toward product registration. Even for commercialized products, their ultimate use can still be limited by suboptimal performance.

For example, in oncology, with so few therapeutics approved for cancer patients and the realization that cancer is a collection of diseases with a multitude of etiologies, biological phenotypes or genotype with high rise for drug resistance and susceptible genomic mutations and that a patient's response and survival from therapeutic intervention is complex with many factors playing a role in the success or failure of treatment including disease indication, pathology stage related to invasion and metastatic spread, patient gender, age, health conditions, previous therapies or other illnesses, the genetic background of both the patient and the malignancy, and other relevant factors, the opportunity for significant cure rates without treatment morbidity in the near term remains elusive. Moreover, the incidence of cancer continues to rise such that over 1.6 million new cancer cases are estimated for 2015 in the United States by the American Cancer Society. In addition, with advances in diagnosis such as BRCA genetic testing and mammography for breast cancer and PSA tests for prostate cancer, as well as additional tests based on molecular markers, more patients are being diagnosed at a younger age. For difficult to treat cancers, a patient's treatment options are often exhausted quickly resulting in a desperate need for additional treatment regimens. Even for the most limited of patient populations, any additional treatment opportunities would be of considerable value. This invention focuses on inventive compositions and methods for improving the therapeutic benefit of suboptimally administered therapeutic agents including substituted camptothecins such as irinotecan and topotecan.

Therefore, there is a substantial need for improved methods, formulations, and compositions employing substituted camptothecins such as, but not limited to, irinotecan and topotecan for the treatment of malignancies and other diseases and conditions including, but not limited to, non-malignant proliferative disorders, infections, inflammatory, and immunological diseases.

### SUMMARY OF THE INVENTION

The present invention meets the needs described above by providing improved methods, formulations, and compositions employing substituted camptothecins such as, but not limited to, irinotecan and topotecan. These methods, formulations, and compositions can be used to treat malignancies and other diseases and conditions including, but not limited to, non-malignant proliferative disorders, infections, inflammatory, and immunological diseases.

One aspect of the invention is a method to improve the efficacy and/or reduce the side effects of the administration of irinotecan, topotecan, or a derivative or analog of irinotecan or topotecan for treatment of benign or neoplastic hyperproliferative diseases, infections, inflammatory disease or conditions, or immunological diseases or conditions comprising the steps of:
(1) identifying at least one factor or parameter associated with the efficacy and/or occurrence of side effects of the administration of the irinotecan, topotecan, or the derivative or analog of irinotecan or topotecan for the treatment of benign or neoplastic hyperproliferative diseases, infections, inflammatory disease or conditions, or immunological diseases; and
(2) modifying the factor or parameter to improve the efficacy and/or reduce the side effects of the administration of the irinotecan, topotecan, or the derivative or analog of irinotecan or topotecan for the treatment of benign or neoplastic hyperproliferative diseases, infections, inflammatory diseases or conditions, or immunological diseases.

Typically, the factor or parameter is selected from the group consisting of:
(1) dose modification;
(2) route of administration;
(3) schedule of administration;
(4) indications for use;
(5) disease stages;
(6) other indications;
(7) patient selection;
(8) patient or disease phenotype;
(9) patient or disease genotype;
(10) pre-post/treatment preparation
(11) toxicity management;
(12) pharmacokinetic/pharmacodynamic monitoring;
(13) drug combinations;
(14) chemosensitization;
(15) chemopotentiation;
(16) post-treatment management;
(17) alternative medicine/therapeutic support;
(18) bulk drug product improvements;
(19) diluent systems;
(20) solvent systems;
(21) excipients;
(22) dosage forms;
(23) dosage kits and packaging;
(24) drug delivery systems;
(25) drug conjugate forms;
(26) compound analogs;
(27) prodrug systems;
(28) multiple drug systems;
(29) biotherapeutic enhancement;
(30) biotherapeutic resistance modulation;
(31) radiation therapy enhancement;
(32) novel mechanisms of action;
(33) selective target cell population therapeutics;
(34) use of liposomes for drug delivery;
(35) use of crystalline polymorphisms; and
(36) use of stereoisomers.

Typically, the topotecan, or the derivative or analog of irinotecan or topotecan is irinotecan or topotecan.

Typically, the method treats a neoplastic hyperproliferative disease. Typically, the neoplastic hyperproliferative disease is selected from the group consisting of colorectal cancer, pancreatic cancer, lung cancer, breast cancer, gastric cancer, locally advanced or metastatic breast cancer, ovarian cancer, rhabdomyosarcoma, cervical cancer, neuroblastoma, glioblastoma multiforme, Ewing's sarcoma, non-Hodgkin's lymphoma, endometrial cancer, and oligodendroglioma. As stated above, methods according to the present invention can also be used to treat other non-malignant conditions, such as, but not limited to, benign hyperproliferative diseases, infections, inflammatory diseases or conditions, or immunological diseases.

Another aspect of the invention is a composition to improve the efficacy or reduce the side effects of treatment with irinotecan, topotecan, or a derivative, analog, salt, solvate or prodrug of irinotecan or topotecan wherein the composition comprises:
(a) an alternative selected from the group consisting of:
   (i) a therapeutically effective quantity of irinotecan, topotecan, or a derivative, analog, prodrug, salt, or solvate of irinotecan or topotecan;
   (ii) two or more therapeutically active ingredients comprising:
      (A) a therapeutically effective quantity of irinotecan, topotecan, or a derivative, analog, salt, or solvate of irinotecan or topotecan; and
      (B) at least one additional therapeutic agent, therapeutic agent subject to chemosensitization, therapeutic agent subject to chemopotentiation, or component of a multiple drug system;
   (iii) a therapeutically effective quantity of irinotecan, topotecan, or a derivative, analog, prodrug, salt, or solvate of irinotecan or topotecan that is incorporated into a dosage form;
   (iv) a therapeutically effective quantity of irinotecan, topotecan, or a derivative, analog, prodrug, salt, or solvate of irinotecan or topotecan that is incorporated into a dosage kit and packaging;
   (v) a therapeutically effective quantity of irinotecan, topotecan, or a derivative, analog, prodrug, salt, or solvate of irinotecan or topotecan that is subjected to a bulk drug product improvement;
   (vi) a therapeutically effective quantity of irinotecan, topotecan, or a derivative, analog, prodrug, salt, or solvate of irinotecan or topotecan that is incorporated into a drug delivery system;
   (vii) a therapeutically effective quantity of a prodrug of irinotecan or topotecan or a derivative or analog of irinotecan or topotecan; and
   (viii) a therapeutically effective quantity of irinotecan, topotecan, or a derivative, analog, prodrug, salt, or solvate of irinotecan or topotecan that is incorporated into a liposomal formulation; and
(b) at least one pharmaceutically acceptable diluent, solvent or excipient.

### Definitions

Although any methods and materials similar to or equivalent to those described herein can be used in the practice or testing of embodiments described herein or other embodiments within the scope of the invention, some preferred methods, compositions, materials, and devices are described herein. However, in this context, it must be understood that this invention is not limited to the particular molecules, compositions, methodologies, or protocols described herein, as these aspects of the invention may vary in accordance with routine experimentation and optimization as is generally known in the art. It is also to be understood that the terminology used in the description and the claims is for the purpose of describing the particular versions or embodiments only, and is not intended to limit the scope of the embodiments as described herein as understood by one of skill in the art.

Unless otherwise defined, all technical and scientific terms used herein have the same meanings as commonly understood by one of ordinary skill in the art to which this invention belongs. However, in case of any conflict of meanings, the present specification and claims, including definitions therein, shall control. Accordingly, in the context of the embodiments described herein, the following definitions apply.

As used herein and in the appended claims, the singular forms “a,” “an,” and “the” include references to the plural unless the context clearly dictates otherwise. Thus, for example, a reference to “a PARP inhibitor” is a reference to one or more PARP inhibitors or equivalents thereof known to those skilled in the art.

As used herein, the terms “comprise,” “include,” and linguistic variations thereof denote the presence of recited features, elements, method steps, or other components of the invention without the exclusion of the presence of additional/recited features, elements, method steps, or other components. Conversely, the terms “consisting of” and linguistic variations thereof denote the presence of recited features, elements, method steps, or other components of the invention and exclude any unrecited recited features, elements, method steps, or other components of the invention except for ordinarily-associated impurities. The phrase “consisting essentially of” and linguistic variations thereof denote the presence of recited features, elements, method steps, or other components of the invention and any additional features, elements, method steps, or other components of the invention that do not materially affect the basic nature of the composition, system, or method. Many embodiments herein are described using open “comprising” language; such embodiments also encompass embodiments described in terms of “consisting essentially of” or “consisting of” language, which may be alternatively claimed or described using such language, unless the context clearly excludes “consisting essentially of” or “consisting of” language.

All chemical names used herein, including names of substituents, should be interpreted in light of the chemical nomenclature conventions of IUPAC and/or a modified format in which functional groups within a substituent are read in the order in which they branch from the scaffold or main structure. For example, in the modified nomenclature, methylsulfonylpropanol refers to CH₂SO₂CH₂CH₂CH₂OH or

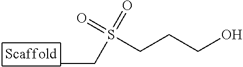

As another example, according to the modified nomenclature, a methylamine substituent is

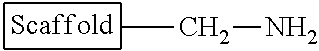

while an aminomethyl substituent is

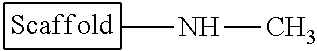

As used herein, the term “subject” broadly refers to any animal, including, but not limited to, humans and non-human mammals. The reference to non-human mammals includes, but is not limited to, socially or economically important animals or animals used for research including cattle, sheep, goats, horses, pigs, llamas, alpacas, dogs, cats, rabbits, guinea pigs, rats, and mice. Unless specified, methods and compositions according to the present invention are not limited to treatment of humans. In general, when treatment of humans is intended, the term “patient” can used in place of “subject.”

As used herein, the terms “effective amount,” “therapeutically effective amount,” or other equivalent terminology refer to the amount of a compound or compounds or to the amount of a composition sufficient to effect beneficial or desired results. The beneficial or desired results are typically a reduction in severity, symptoms, or duration of a disease or condition being treated and can generally be characterized as an amount of a therapeutic agent or composition effective to treat, ameliorate, or prevent a desired disease or condition, or to exhibit a detectable therapeutic or preventative effect. The use of such terminology cannot, unless specifically indicated, be interpreted as implying a complete cure for any disease or condition as recited herein. An effective amount can be administered in one or more administrations, applications, or dosages, and is not intended to be limited to a particular formulation or administration route unless a particular formulation or administration route is specified. The effect induced by the administration of a therapeutically effective amount can be detected by, for example, chemical markers, antigen levels, or changes in pathological indicators such as tumor burden. Therapeutic effects also can include subjective improvements in well-being, reduction of fatigue, or increased energy noted by the subjects or their caregivers. For example, when the therapeutic agent is administered to treat a malignancy, a “beneficial clinical outcome” can include, but is not necessarily limited to: a reduction in tumor mass or tumor burden; a reduction in tumor spread or metastasis; a reduction in pain; a reduction of symptoms associated with the malignancy such as seizures for central nervous system malignancies; a reduction of fatigue; a reduction of malaise; an increase in longevity; or an improved Karnofsky performance score. The precise therapeutically effective amount for a subject will depend upon the subject's size, weight, and health, the nature and extent of the condition affecting the subject, the administration of other therapeutics administered to treat the particular disease or condition being treated or other diseases or conditions affecting the subject, as well as variables such as liver and kidney function that affect the pharmacokinetics of administered therapeutics. Thus, it is not useful to specify an exact effective amount in advance. However, the therapeutically effective amount for a given situation can be determined by routine experimentation and is within the judgment of the clinician.

As used herein, the terms “administration,” “administering,” or other equivalent terminology, refer to the act of giving a drug, prodrug, pharmaceutical composition, or other agent intended to provide therapeutic treatment to a subject or in vivo, in vitro, or ex vivo to cells, tissues, or organs. Exemplary routes of administration to the human body can be through space under the arachnoid membrane of the brain or spinal cord (intrathecal), the eyes (ophthalmic), mouth (oral), skin (topical or transdermal), nose (nasal), lungs or other portions of the respiratory tract (inhalant), oral mucosa (buccal), ear, rectal, vaginal, by injection (such as, but not limited to, intravenously, subcutaneously, intraperitoneally, or by other injection routes as known in the art).

As used herein, the terms “co-administration,” “co-administering,” or equivalent terminology refer to the administration of at least two agents, such as, for example, irinotecan, topotecan, or a derivative or analog thereof and a PARP inhibitor, or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful agent or agent, and/or when co-administration of two or more agents results in sensitization of a subject to beneficial effects of one of the agents via co-administration of the other agent. As used herein, the term “concurrent administration” refers to the administration of two or more active agents sufficiently close in time to achieve a combined therapeutic effect that is preferably greater than that which would be achieved by the administration of either agent alone. Such concurrent administration can be carried out simultaneously, e.g., by administering the active agents together in a common pharmaceutically acceptable carrier, thereby forming a pharmaceutical composition with two or more active agents, in one or more doses of the pharmaceutical composition.

As used herein, the term “pharmaceutical composition” refers to the combination of one or more therapeutically active agents with at least one carrier, inert or active, making the composition especially suitable for diagnostic or therapeutic use in vitro, in vivo or ex vivo. Pharmaceutical compositions can be prepared in unit dose form.

As used herein, the terms “pharmaceutically acceptable” or “pharmacologically acceptable,” as used herein, refer to compositions, or components within compositions, that do not substantially produce adverse reactions, such as, but not limited to, toxic, allergic, or unwanted immunological reactions, when administered to a subject.

As used herein, the term “pharmaceutically acceptable carrier” refers to any of the standard pharmaceutical carriers including, but not limited to, phosphate buffered saline solution, water, emulsions, such as oil/water or water/oil emulsions), and various types of wetting agents, any and all solvents, dispersion media, coatings, sodium lauryl sulfate, isotonic and absorption delaying agents, disintegrants such as potato starch or sodium starch glycolate), and the like. The carriers also can include stabilizers and preservatives.

As used herein, the term “pharmaceutically acceptable salt” refers to any pharmaceutically acceptable salt (e.g., acid or base) of a compound that is used in a method of the present invention or is a component of a composition of the present invention, which, upon administration to a subject, is capable of providing a compound of the present invention or an active metabolite or residue thereof. As is known to those of skill in the art, salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and other acids known in the art as suitable for formation of pharmaceutically acceptable salts. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (such as sodium or potassium) hydroxides, alkaline earth metals (such as calcium or magnesium), hydroxides, ammonia, and compounds of formula NW₄⁺, wherein W is C₁-C₄ alkyl, and the like. Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺, wherein W is a C₁-C₄ alkyl group), and the like. For therapeutic use, salts of the compounds herein are contemplated as being pharmaceutically acceptable. However, salts of acids and bases that are non-pharmaceutically acceptable may also find use, for example, in the preparation or purification of a pharmaceutically acceptable compound.

As used herein, the term “instructions for administering a compound to a subject,” and grammatical equivalents thereof, includes instructions for using the compositions contained in a kit for the treatment of conditions. Such instructions, for example, provide dosing, routes of administration, or decision trees for treating physicians for correlating patient-specific characteristics with therapeutic courses of action. Such instructions may be part of a kit according to the present invention.

The following applies to analogs and derivatives of the compounds described in further detail below, including irinotecan, topotecan, and other therapeutically active agents described herein. As used herein, “analog” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or hydrophobic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers. As used herein, “derivative” refers to a chemically or biologically modified version of a chemical compound that is structurally similar to a parent compound and (actually or theoretically) derivable from that parent compound. A “derivative” differs from an “analog” in that a parent compound may be the starting material to generate a “derivative,” whereas the parent compound may not necessarily be used as the starting material to generate an “analog.” A derivative may or may not have different chemical or physical properties than the parent compound. For example, the derivative may be more hydrophilic or hydrophobic or it may have altered reactivity as compared to the parent compound. Derivatization (i.e., modification) may involve substitution of one or more moieties within the molecule (e.g., a change in functional group). The term “derivative” also includes conjugates and prodrugs of a parent compound (i.e., chemically modified derivatives which can be converted into the original compound under physiological conditions).

As used herein, the term “alkyl” refers to an unbranched, branched, or cyclic saturated hydrocarbyl residue, or a combination thereof, of from 1 to 12 carbon atoms, or in some cases up to 50 or more carbon atoms, that can be optionally substituted; the alkyl residues contain only C and H when unsubstituted. Typically, the unbranched or branched saturated hydrocarbyl residue is from 1 to 6 carbon atoms, preferably 1 to 4 carbon atoms, more preferably 1 to 3 carbon atoms, which is referred to herein as “lower alkyl.” When the alkyl residue is cyclic and includes a ring, it is understood that the hydrocarbyl residue includes at least three carbon atoms, which is the minimum number to form a ring. An alkyl group can be linear, branched, cyclic, or a combination thereof, and may contain from 1 to 50 or more carbon atoms, such as a straight chain or branched C₁-C₂₀ alkane. Examples of alkyl groups include but are not limited to methyl, ethyl, propyl, isopropyl, cyclopropyl, butyl isomers (e.g. n-butyl, isobutyl, and tert-butyl), cyclobutyl isomers (e.g. cyclobutyl, methylcyclopropyl, etc.), pentyl isomers, cyclopentane isomers, hexyl isomers, cyclohexane isomers, and the like. Unless specified otherwise (e.g., substituted alkyl group, heteroalkyl, alkoxy group, haloalkyl, alkylamine, thioalkyl, etc.), an alkyl group contains carbon and hydrogen atoms only. As used herein, the term “linear alkyl” refers to a chain of carbon and hydrogen atoms (e.g., ethane, propane, butane, pentane, hexane, or other examples). A linear alkyl group may be referred to by the designation —(CH₂)_{q}CH₃, where q is 0-49. The designation “C₁-C₁₂ alkyl” or a similar designation refers to alkyl having from 1 to 12 carbon atoms such as methyl, ethyl, propyl isomers (e.g. n-propyl or isopropyl), butyl isomers, cyclobutyl isomers (e.g. cyclobutyl or methylcyclopropyl), pentyl isomers, cyclopentyl isomers, hexyl isomers, cyclohexyl isomers, heptyl isomers, cycloheptyl isomers, octyl isomers, cyclooctyl isomers, nonyl isomers, cyclononyl isomers, decyl isomers, cyclodecyl isomers, or other alternatives known in the art. Similar designations refer to alkyl with a number of carbon atoms in a different range. As used herein, the term “Cₓ-C_{y}” when used in conjunction with a chemical moiety, such as alkyl, alkenyl, alkynyl, or carbocycle is meant to include groups that contain from x to y carbons in the chain or ring. For example, the term “Cₓ-C_{y} alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, or other alternatives. The terms “Cₓ-C_{y} alkenyl” and “Cₓ-C_{y} alkynyl” refer to substituted or unsubstituted unsaturated aliphatic groups analogous in length and possible substitution to the alkyls described above, but that contain at least one double or triple bond respectively. The term “Cₓ-C_{y} carbocycle” refers to a substituted or unsubstituted carbocycle, that contain from x to y ring carbons. As used herein, the term “branched alkyl” refers to a chain of carbon and hydrogen atoms, without double or triple bonds, that contains a fork, branch, and/or split in the chain (e.g., 3,5-dimethyl-2-ethylhexane, 2-methyl-pentane, 1-methyl-cyclobutane, ortho-diethyl-cyclohexane, or other alternatives). “Branching” refers to the divergence of a carbon chain, whereas “substitution” refers to the presence of non-carbon/non-hydrogen atoms in a moiety. Unless specified otherwise (e.g., substituted branched alkyl group, branched heteroalkyl, branched alkoxy group, branched haloalkyl, branched alkylamine, branched thioalkyl, or other alternatives), a branched alkyl group contains carbon and hydrogen atoms only.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocycle can be fully saturated or partially saturated, but non-aromatic. For example, the general term “carbocyclyl” encompasses cycloalkyl. The carbocyclic and heterocyclic structures encompass compounds having monocyclic, bicyclic or multiple (polycyclic) ring systems; and such systems may mix aromatic, heterocyclic, and carbocyclic rings. Mixed ring systems are described according to the ring that is attached to the rest of the compound being described. Bicyclic or polycyclic rings may include fused or spiro rings. Carbocycles may include 3- to 10-membered monocyclic rings, 6- to 12-membered bicyclic rings, and 6- to 12-membered bridged rings. Each ring of a bicyclic or polycyclic carbocycle may be selected from saturated, unsaturated, and aromatic rings. In an exemplary embodiment, an aromatic carbocycle, e.g., phenyl, may be fused to a saturated or unsaturated ring, e.g., cyclohexane, cyclopentane, or cyclohexene. In some embodiments, the carbocycle is an aromatic carbocycle. In some embodiments, the carbocycle is a cycloalkyl. In some embodiments, the carbocycle is a cycloalkenyl. Exemplary carbocycles include cyclopentyl, cyclohexyl, cyclohexenyl, adamantyl, phenyl, indanyl, and naphthyl. An alkenyl group can be optionally substituted by one or more substituents such as those substituents described herein. A “non-aromatic carbocycle” includes rings and ring systems that are saturated, unsaturated, substituted or unsubstituted, but not aromatic or aryl rings or ring systems.

As used herein, the term “cycloalkyl” refers to a completely saturated mono- or multi-cyclic hydrocarbon ring system. When composed of two or more rings, the rings may be joined together in a fused, bridged or spiro-connected fashion. Cycloalkyl groups of the present application may range from three to ten carbons (C₃ to C₁₀). A cycloalkyl group may be unsubstituted, substituted, branched, and/or unbranched. Typical cycloalkyl groups include, but are not limited to, cyclopropyl, cyclobutyl, cyclopentyl, cyclohexyl, and the like. If substituted, the substituent(s) may be an alkyl or can be selected from those indicated above with regard to substitution of an alkyl group unless otherwise indicated. While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

As used herein, the term “heteroalkyl” refers to an alkyl group, as defined herein, wherein one or more carbon atoms are independently replaced by one or more heteroatoms (e.g., oxygen, sulfur, nitrogen, phosphorus, selenium, silicon, or combinations thereof). The alkyl group containing the non-carbon substitution(s) may be a linear alkyl, branched alkyl, cycloalkyl (e.g., cycloheteroalkyl), or combinations thereof. Non-carbons may be at terminal locations (e.g., 2-hexanol) or integral to an alkyl group (e.g., diethyl ether). In general, the “hetero” terms refer to groups that typically contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. In some cases, more than three heteroatoms may be present. Unless stated otherwise specifically in the specification, the heteroalkyl group may be optionally substituted as described herein. Representative heteroalkyl groups include, but are not limited to —OCH₂OMe, —OCH₂CH₂OMe, or —OCH₂CH₂OCH₂CH₂NH₂. For reasons of chemical stability, it is also understood that, unless otherwise specified, such groups do not include more than two contiguous heteroatoms except where an oxo group is present on N or S as in a nitro or sulfonyl group.

As used herein, the term “heteroalkylene” refers to an alkyl radical as described above where one or more carbon atoms of the alkyl is replaced with a heteroatom, e.g., O, N or S, or another heteroatom as described above. “Heteroalkylene” or “heteroalkylene chain” refers to a straight or branched divalent heteroalkyl chain linking the rest of the molecule to a radical group. Unless stated otherwise specifically in the specification, the heteroalkylene group may be optionally substituted as described herein. Representative heteroalkylene groups include, but are not limited to —OCH₂CH₂O—, —OCH₂CH₂OCH₂CH₂O—, or —OCH₂CH₂OCH₂CH₂OCH₂CH₂O—.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule and such that a stable compound is formed thereby, i.e., a compound that does not spontaneously undergo transformation such as by rearrangement, cyclization, elimination, hydrolysis, lactone or lactam formation, or other reaction. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents. Substitution of a structure depicted herein may result in removal or moving of a double bond or other bond, as will be understood by one in the field. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds that do not significantly alter the pharmacological activity of the compound in the context of the present invention. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. The heteroatoms such as nitrogen may have hydrogen substituents and/or any permissible substituents of organic compounds described herein which satisfy the valences of the heteroatoms.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, or I). When an alkyl group is substituted with more than one halogen radical, each halogen may be independently selected e.g., 1-chloro, 2-fluoroethane.

As used herein, the term “aryl” refers to a monocyclic or fused bicyclic moiety having the well-known characteristics of aromaticity; examples include phenyl and naphthyl, which can be optionally substituted. Additional examples of aromatic rings include furan, benzofuran, isobenzofuran, pyrrole, indole, isoindole, thiophene, benzothiophene, benzo(c)thiophene, imidazole, benzimidazole, purine, pyrazole, indazole, oxazole, benzooxazole, isoxazole, benzisoxazole, thiazole, benzothiazole, benzene, naphthalene, pyridine, quinolone, isoquinoline, pyrazine, quinoxaline, pyrimidine, quinazoline, pyridazine, cinnoline, phthalazine, triazine (e.g., 1,2,3-triazine; 1,2,4-triazine; 1,3,5 triazine), and thiadiazole. The term “aromatic carbocycle” refers to an aromatic ring without heteroatoms present within the ring structure, such as, but not limited to benzene or naphthalene. Other terms that can be used include “aromatic ring,” “aryl group,” or “aryl ring.”

As used herein, the term “heterocycle,” “heterocyclyl,” “heterocyclic ring” or “heterocyclic group” is intended to mean a stable 4-, 5-, 6-, or 7-membered monocyclic or 7-, 8-, 9-, 10-, 11-, 12-, 13-, or 14-membered bicyclic heterocyclic ring which is saturated, partially unsaturated, or fully unsaturated or aromatic, and which consists of carbon atoms and 1, 2, 3 or 4 heteroatoms independently selected from N, O, and S; and including any bicyclic group in which any of the above-defined heterocyclic rings is fused to a benzene ring. Other heteroatoms, such as P, Se, B, or Si, can be included in some alternatives. The nitrogen and sulfur heteroatoms may optionally be oxidized. The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. When the term “heterocycle,” “heterocyclyl,” “heterocyclic ring” or “heterocyclic group” is used, it is intended to include heteroaryl unless heteroaryl is excluded. Examples of heterocycles include, but are not limited to, acridinyl, azocinyl, benzimidazolyl, benzofuranyl, benzothiofuranyl, benzoxazolyl, benzoxazolinyl, benzthiazolyl, benztriazolyl, benztetrazolyl, benzisoxazolyl, benzisothiazolyl, benzimidazolinyl, carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

As used herein, the term “non-aromatic heterocycle” refers to a cycloalkyl or cycloalkenyl, as defined herein, wherein one or more of the ring carbons are replaced by a moiety selected from —O—, —N═, —NR—, —C(O)—, —S—, —S(O)— or —S(O)₂—, wherein R is hydrogen, C₁-C₈ alkyl or a nitrogen protecting group, with the proviso that the ring of such a group does not contain two adjacent O or S atoms. In some alternatives, other heteroatoms including P, Se, B, or Si can be included. Non-limiting examples of non-aromatic heterocycles, as used herein, include morpholino, pyrrolidinyl, pyrrolidinyl-2-one, piperazinyl, piperidinyl, piperidinylone, 1,4-dioxa-8-aza-spiro(4.5)dec-8-yl, 2H-pyrrolyl, 2-pyrrolinyl, 3-pyrrolinyl, 1,3-dioxolanyl, 2-imidazolinyl, imidazolidinyl, 2-pyrazolinyl, pyrazolidinyl, 1,4-dioxanyl, 1,4-dithianyl, thiomorpholinyl, azepanyl, hexahydro-1,4-diazepinyl, tetrahydrofuranyl, dihydrofuranyl, tetrahydrothienyl, tetrahydropyranyl, dihydropyranyl, tetrahydrothiopyranyl, thioxanyl, azetidinyl, oxetanyl, thietanyl, oxepanyl, thiepanyl, 1,2,3,6-tetrahydropyridinyl, 2H-pyranyl, 4H-pyranyl, dioxanyl, 1,3-dioxolanyl, dithianyl, dithiolanyl, dihydropyranyl, dihydrothienyl, dihydrofuranyl, imidazolinyl, imidazolidinyl, 3-azabicyclo(3.1.0)hexanyl, and 3-azabicyclo(4.1.0)heptanyl, 3,8-diazabicyclo(3.2.1)octanyl, and 2,5-diazabicyclo(2.2.1)heptanyl. In certain embodiments, a non-aromatic heterocyclic ring is aziridine, thiirane, oxirane, oxaziridine, dioxirane, azetidine, oxetan, thietane, diazetidine, dioxetane, dithietane, pyrrolidine, tetrahydrofuran, thiolane, imidazolidine, pyrazolidine, oxazolidine, isoxazolidine, thiazolidine, isothiazolidine, dioxolane, dithiolane, piperdine, oxane, thiane, piperazine, morpholine, thiomorpholine, dioxane, dithiane, trioxane, thithiane, azepane, oxepane, thiepane, homopiperazine, or azocane.

As used herein, the terms “heteroaryl” or “heteroaromatic” refer to monocyclic, bicyclic, or polycyclic ring systems, wherein at least one ring in the system is aromatic and contains at least one heteroatom, for example, nitrogen, oxygen and sulfur. Each ring of the heteroaromatic ring systems may contain 3 to 7 ring atoms. Exemplary heteroaromatic monocyclic ring systems include 5- to 7-membered rings whose ring structures include one to four heteroatoms, for example, one or two heteroatoms. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxyheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. When a range of values is listed, such as for the number of carbon atoms in an alkyl group, it is intended to encompass each value and subrange within the range. For example, “C₁-C₆ alkyl” includes alkyl groups with 1, 2, 3, 4, 5, or 6 carbon atoms and all possible subranges.

As used herein, the term “hydroxyaryl” refers to an aryl group including one or more hydroxyl groups as substituents; as further detailed below, further substituents can be optionally included.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. The term “solvate” typically means a physical association of a compound involving varying degrees of ionic and/or covalent bonding, including hydrogen bonding. In certain instances the solvate will be capable of isolation, for example when one or more solvent atoms are incorporated into the crystal lattice of the crystalline solid. The term “solvate” encompasses both solution-phase and isolatable solvates. Suitable solvates in which the solvent is other than water include, but are not limited to, ethanolates or methanolates. When water is the solvent, the corresponding solvate is a “hydrate.” Examples of hydrates include, but are not limited to, hemihydrate, monohydrate, dihydrate, trihydrate, hexahydrate, and other hydrated forms. It should be understood by one of ordinary skill in the art that the pharmaceutically acceptable salt and/or prodrug of compounds described herein for use in methods or compositions according to the present invention may also exist in a solvate form. When the solvate is a hydrate, the hydrate is typically formed via hydration which is either part of the preparation of the present compound or through natural absorption of moisture by the anhydrous compound of the present invention. Additionally, compounds may exist as clathrates or other complexes, which are therapeutic agent-host inclusion complexes wherein the therapeutic agent and the host are present in stoichiometric or non-stoichiometric amounts.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolyzable ester groups. That is, these esters are pharmaceutically acceptable and can be hydrolyzed to the corresponding carboxylic acid in vivo.

As used herein, the term “alkenyl” refers to an unbranched, branched or cyclic hydrocarbyl residue having one or more carbon-carbon double bonds. Typically, the hydrocarbyl residue has from 2 to 12 carbon atoms (C₂-C₁₂ alkenyl). In certain embodiments, an alkenyl comprises two to eight carbon atoms (C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. An alkenyl group can be optionally substituted by one or more substituents such as those substituents described herein. With respect to the use of “alkenyl,” the presence of multiple double bonds cannot produce an aromatic ring structure.

As used herein, the term “alkynyl” refers to an unbranched, branched, or cyclic hydrocarbyl residue having one or more carbon-carbon triple bonds; the residue can also include one or more double bonds. Typically, the hydrocarbyl residue has from 2 to 12 carbon atoms (C₂-C₁₂ alkynyl). In certain embodiments, an alkenyl comprises two to eight carbon atoms (C₂-C₈ alkynyl). In certain embodiments, an alkenyl comprises two to six carbon atoms (i.e., C₂-C₆ alkynyl). In other embodiments, an alkenyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. With respect to the use of “alkynyl,” the presence of multiple double bonds in addition to the one or more triple bonds cannot produce an aromatic ring structure.

As used herein, the term “alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises only one carbon atom (i.e., C₁ alkylene or a —CH₂— group). An alkylene group can be optionally substituted by one or more substituents such as those substituents described herein.

As used herein, the term “alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). An alkenylene group can be optionally substituted by one or more substituents such as those substituents described herein.

As used herein “alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). An alkenylene group can be optionally substituted by one or more substituents such as those substituents described herein.

As used herein, the term “amine” or “amino” includes primary, secondary, and tertiary amines wherein each non-hydrogen group on nitrogen may be selected from alkyl, aryl, and the like. Amines include but are not limited to —NH₂, —NH-phenyl, —NH—CH₃, —NH—CH₂CH₃, and —N(CH₃)benzyl. The amino group can be optionally substituted. For example, the term can include NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “amide” or “amido” includes C- and N-amide groups, e.g., —C(O)NR₂, and —NRC(O)R groups, respectively, where R can be H, alkyl, aryl, or other groups, which can be optionally substituted. Amide groups therefore include but are not limited to —C(O)NH₂, —NHC(O)H, —C(O)NHCH₂CH₃, —NHC(O)CH₃, or —C(O)N(CH₂CH₃)phenyl.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

As used herein, similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

As used herein, the term “heteroatom” refers to any atom that is not carbon or hydrogen, such as nitrogen, oxygen, phosphorus, or sulfur. When it is part of the backbone or skeleton of a chain or ring, a heteroatom must be at least divalent, and will typically be selected from N, O, P, and S, more typically from N, O, and P. The term “heteroatom” can include, in some contexts, other atoms, including selenium, silicon, or boron.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

As used herein, the term “absent” when used in reference to a functional group or substituent, particularly in reference to the chemical structure of a compound, means that the particular functional group or substituent is not present in the compound being described. When used in reference to a substituent, the absence of the substituent typically means that the bond to the substituent is absent and that absence of the bond is compensated for with a H atom. When used in reference to a position within a chain or ring, the absence of the position typically means that the two positions otherwise connected by the absent position are instead directly connected by a covalent bond.

As used herein, the term “PEG” or “polyethylene glycol” refers to any polyethylene oxide moiety, typically water-soluble. Typically, PEGs for use in the present invention will comprise one of the two following structures: “—(CH₂CH₂O)ₙ”— or “—(CH₂CH₂O)ₙ₋₁CH₂CH₂—,” depending upon whether or not the terminal oxygen(s) has been displaced.

As used herein, the term “water-soluble” in the context of a polymer described herein as employed herein in a method or composition according to the present invention, is any segment or polymer that is soluble in water at room temperature. Typically, a water-soluble polymer or segment will transmit at least about 75%, more preferably at least about 95% of light, transmitted by the same solution after filtering. On a weight basis, a water-soluble polymer or segment thereof will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer or segment is about 95% (by weight) soluble in water or completely soluble in water.

As used herein, the term “linker” refers to a group or moiety used to link interconnected moieties, such as, but not limited to, irinotecan, topotecan, or a derivative or analog thereof that is linked with another drug, a delivery agent, a polymer, or another group or moiety that can modulate the pharmacological activity of the irinotecan, topotecan, or derivative or analog thereof. A linker group or moiety may be hydrolytically stable or may include a physiologically hydrolysable or enzymatically hydrolysable linkage.

A hydrolysable bond is a covalent bond that reacts with water (i.e., is hydrolyzed) under physiological conditions. The tendency of a bond to hydrolyze in water depends not only on the general type of linkage linking the two atoms wherein the bond between the two atoms is hydrolyzed but also on the substituents attached to those two atoms. Illustrative hydrolytically unstable linkages include, but are not limited to, carboxylate esters, phosphate esters, anhydrides, acetals, ketals, acyloxyalkyl ethers, imines, orthoesters, peptides, and oligonucleotides.

An enzymatically degradable linkage is a linkage that is subject to degradation by one or more enzymes.

A hydrolytically stable linkage is a chemical bond, typically a covalent bond, that is substantially stable in an aqueous medium and that does not undergo hydrolysis under physiological conditions to any appreciable extent over an extended period of time. Examples of hydrolytically stable linkages include but are not limited to: carbon-carbon bonds such as in aliphatic chains, ethers, amides, or urethanes. Typically, a hydrolytically stable linkage is one that exhibits a rate of hydrolysis of less than about 1-2% per day under physiological conditions. The designation of a linkage as a hydrolytically stable linkage does not exclude the possibility of enzymatically-catalyzed hydrolysis of the linkage by a specific enzyme or enzymes.

As used herein in the context of a polymer containing multiple copies of an irinotecan, topotecan, or derivative or analog thereof, the term “multi-armed” refers to a polymer that has three or more copies of the irinotecan, topotecan, or derivative or analog thereof. The polymer can be a dendritic polymer (dendrimer).

As used herein, the term “antibody,” unless further defined or limited, encompasses both polyclonal and monoclonal antibodies, as well as genetically engineered antibodies such as chimeric, humanized or fully human antibodies of the appropriate binding specificity. As used herein, unless further defined or limited so that only complete antibody molecules are intended, the term “antibody” also encompasses antibody fragments such as sFv, Fv, Fab, Fab′ and F(ab)′₂ fragments. In many cases, it is preferred to use monoclonal antibodies. In some contexts, antibodies can include fusion proteins comprising an antigen-binding site of an antibody, and any other modified immunoglobulin molecule comprising an antigen recognition site (i.e., antigen-binding site) as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins, therapeutic agents, antimetabolites, or radioisotopes; in some cases, conjugation occurs through a linker or through noncovalent interactions such as an avidin-biotin or streptavidin-biotin linkage.

### DETAILED DESCRIPTION OF THE INVENTION

The present invention provides improved methods, formulations, and compositions employing substituted camptothecins such as, but not limited to, irinotecan and topotecan.

### I. Camptothecins

Camptothecin itself has the structure shown in Formula (I):

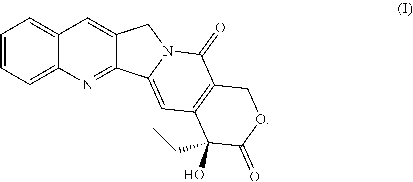 [SMILES: CC[C@@]1(O)C(=O)OCc2c1cc1n(c2=O)Cc2cc3ccccc3nc2-1]

The ring structure of camptothecin together with the conventional numbering of the carbon and nitrogen atoms of the camptothecin structure is shown below as Formula (II).

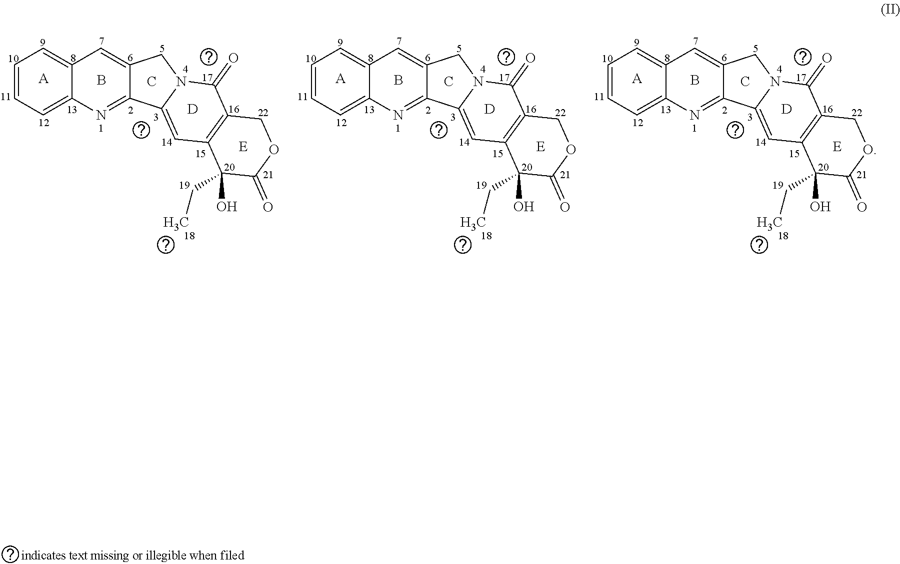

The camptothecin molecule has five fused ring structures; the ring structures are labeled A, B, C, D, and E in Formula (II).

Camptothecins are inhibitors of topoisomerase I. Camptothecin itself had shown anticancer activity in preliminary clinical trials, especially against breast, ovarian, colon, lung, and stomach cancers. However, camptothecin itself has low solubility and adverse effects have been reported when used therapeutically.

Camptothecin has a planar pentacyclic ring structure, that includes a pyrrolo[3,4-β]-quinoline moiety (rings A, B and C), conjugated pyridine moiety (ring D) and one chiral center at position 20 within the α-hydroxy lactone ring with (S) configuration (the E-ring). Its planar structure is thought to be one of the most important factors in topoisomerase inhibition.

Camptothecin binds to the topoisomerase I and DNA complex (the covalent complex) resulting in a ternary complex, and thus stabilizing the ternary complex. This prevents DNA re-ligation, thereby causing DNA damage leading to apoptosis. Camptothecin binds both to the enzyme and DNA with hydrogen bonds. The most important part of the camptothecin structure is the E-ring which interacts from three different positions with the enzyme. The hydroxyl group in position 20 of camptothecin forms a hydrogen bond to the side chain on aspartic acid number 533 (Asp533) in the enzyme. It is critical that the configuration of the chiral carbon is (S) because (R) is inactive. The lactone is bonded with two hydrogen bonds to the amino groups on arginine 364 (Arg364). The D-ring of the camptothecin interacts with the +1 cytosine on the non-cleaved strand and stabilizes the topoisomerase I-DNA covalent complex by forming a hydrogen bond. This hydrogen bond is between a carbonyl group in position 17 on the D-ring and an amino group on the pyrimidine ring of +1 cytosine. Camptothecin is selectively cytotoxic to the cells replicating DNA during S phase and its toxicity is primarily a result of conversion of single-strand breaks into double-strand breaks when the replication fork collides with the cleavage complexes formed by DNA and camptothecin.

One issue with camptothecin is that the lactone ring of the molecule is highly susceptible to hydrolysis with resulting opening of the lactone ring. The resulting open-ring product is inactive. The form with the lactone ring closed is favored under acidic conditions, which prevail in many cancer cell microenvironments. Camptothecin is transported into the cell by passive diffusion. Cellular uptake is favored by lipophilicity, which also makes camptothecin or derivatives thereof more stable as hydrolysis of the lactone ring is avoided. It has been shown that substitutions at positions 7, 9, 10, and 11 of the camptothecin molecule can improve the activity of the molecule as well as its physical properties. Enlargement of the lactone ring by one —CH₂— moiety enhances the activity of the molecule, as in homocamptothecin. Homocamptothecin is shown in Formula (III):

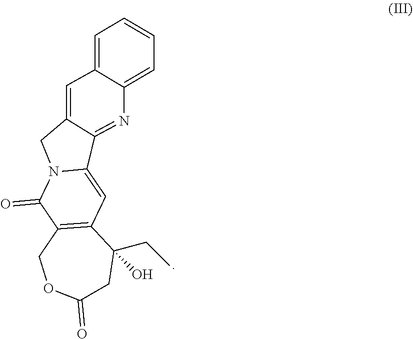 [SMILES: CC[C@@]1(O)CC(=O)OCc2c1cc1n(c2=O)Cc2cc3ccccc3nc2-1]

Other modifications of the original camptothecin structure have been studied. Alkyl substitution at position 7 has shown increased cytotoxicity, such as ethyl (C₂H₅) or chloromethyl (CH₂Cl). These groups are able to react with the DNA in the presence of topoisomerase I which leads to more anti-tumor activity. It has also been shown that increasing the length of the carbon chain (in position 7) leads to increased lipophilicity and consequently greater potency and stability in human plasma. Other 7-modified CPT analogues are silatecans and karenitecins. They are potent inhibitors on topoisomerase I and both have alkylsilyl groups in position 7 which make them lipophilic and more stable. Silatecans or 7-silylcampthothecins have shown reduced drug-HSA interactions which contributes to its blood stability and they can also cross the blood-brain barrier. DB-67 (silatecan) is an active 10-hydroxy derivative. BNP1350 (karenitecin) which belongs to the series of karenitecins exhibits cytotoxic activity and ability to overcome drug resistance. Still another route to make CPTs lipophilic is to introduce lipophilic substituents, such as iminomethyl or oxyiminomethyl moieties. One of the most potent compounds is the oxyiminomethyl derivative ST1481 (gimatecan) that has the advantage of overcoming drug resistance caused by transport systems. The presence of a basic (quaternary) nitrogen in a carbon chain at position 7 makes the compound more hydrophilic and hence more water-soluble. For example, the derivative belotecan (CKD602) is a potent topoisomerase I inhibitor that successfully overcomes the poor water solubility and toxicity seen with camptothecin itself.

In other alternatives, considerably greater activity can be achieved by placing electron-withdrawing groups such as amino, nitro, chloro, or bromo at position 9 or 10 of the camptothecin nucleus and a hydroxyl group at position 10 or 11; however, these compounds are relatively insoluble in aqueous solutions, which can cause difficulty in administration of the compounds. Inclusion of methoxy groups at positions 10 and 11 leads to inactivity.

In other alternatives for camptothecin analogs, hexacyclic camptothecin analogs have been prepared and have shown excellent potency. For example, a methylenedioxy or ethylenedioxy group connected between positions 10 or 11 of the camptothecin structure can form a 5-membered or 6-membered ring which leads to more water-soluble analogs with increased potency. The ethylenedioxy analogs have lower potency than the methylenedioxy analogs, presumably due to the unfavorable steric interactions of the ethylenedioxy analogs with the topoisomerase enzyme.

For these analogs, adding an amino or chloro group at position 9 or a chloromethyl group at position 7 to these 10,11-methylenedioxy or 10,11-ethylenedioxy analogs results in compounds with even greater cytotoxicity but lower solubility in water. In order to improve the water-solubility of these compounds, one alternative is to introduce a water-solubilizing substituent at position 7, as in the analog lurtotecan, which is a 10,11-ethylenedioxy analog with a 4-methylpiperazinomethylene at position 7. The structure of lurtotecan is shown in Formula (IV):

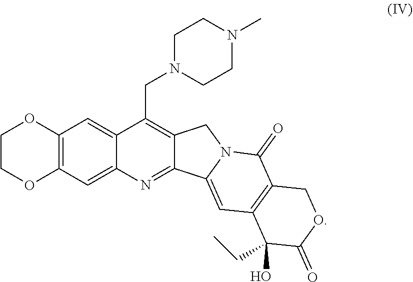 [SMILES: CC[C@@]1(O)C(=O)OCc2c1cc1n(c2=O)Cc2c-1nc1cc3c(cc1c2CN1CCN(C)CC1)OCCO3]

In other alternatives, an additional ring can also be formed between positions 7 and 9 of the camptothecin structure. This can result in further water-soluble derivatives. These hexacyclic camptothecin derivatives demonstrate increased activity when electron-withdrawing groups are placed at position 11 and methyl or amino groups are placed at position 10. Exatecan is an example of a hexacyclic camptothecin derivative that has a six-membered ring between positions 7 and 9 and is also 10-methyl, 11-fluoro substituted. It is water-soluble and is more potent than topotecan. The structure of exatecan is shown below in Formula (V):

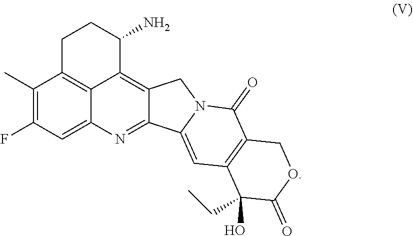 [SMILES: CC[C@@]1(O)C(=O)OCc2c1cc1n(c2=O)Cc2c-1nc1cc(F)c(C)c3c1c2[C@@H](N)CC3]

It has also been shown that the C-ring and D-ring have an essential role in the antitumor activity of camptothecin analogs or derivatives. Replacement in any position results in compounds with much lower potency than the parent compound, camptothecin, in cytotoxicity assays.

With respect to possible modifications in the E-ring of camptothecin, the E-ring does not allow many structural changes without abolishing the topoisomerase I-inhibiting activity of camptothecin because the structure of the E-ring is required for binding to the active site of topoisomerase I. One possible replacement is to replace the hydroxyl group to chloro, bromo, or fluoro because their polarizability is sufficient to stabilize the complex with topoisomerase I. Another possible modification is to insert a methylene group between the hydroxyl group and the lactone group on the E-ring yielding a seven-membered p-hydroxylactone group; this modification results in homocamptothecin, shown above as Formula (III). The hydroxyl of the homocamptothecin has less inductive effect on the carboxyl group which makes the lactone very reactive. This enhances the interaction of the free hydroxyl group with topoisomerase I and the resulting covalent complex is more stable. The E-ring of homocamptothecin opens more slowly and the opening is irreversible. Homocamptothecin and its derivatives exhibit enhanced stability in human plasma due to decreased protein binding and higher affinity for erythrocytes than camptothecin itself.

The structure of silatecan is shown below as Formula (VI):

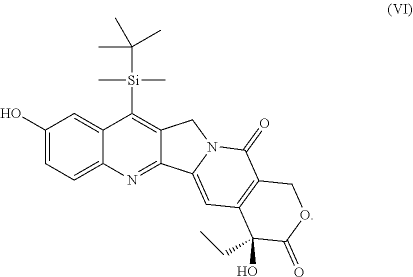 [SMILES: CC[C@@]1(O)C(=O)OCc2c1cc1n(c2=O)Cc2c-1nc1ccc(O)cc1c2[Si](C)(C)C(C)(C)C]

The structure of karenitecan (also known as cositecan) is shown below as Formula (VII).

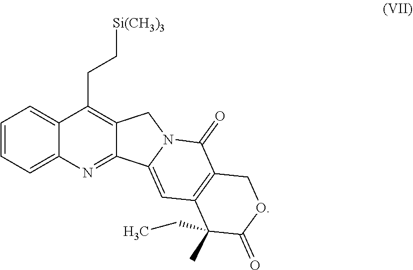

The structure of gimatecan is shown below as Formula (VIII):

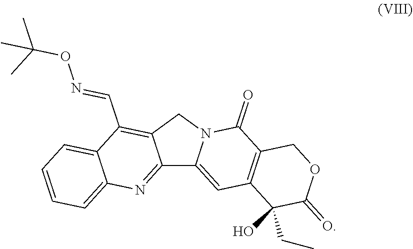 [SMILES: CC[C@@]1(O)C(=O)OCc2c1cc1n(c2=O)Cc2c-1nc1ccccc1c2/C=N/OC(C)(C)C]

The structure of belotecan is shown below as Formula (IX):

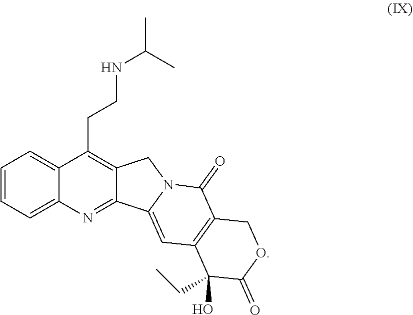 [SMILES: CC[C@@]1(O)C(=O)OCc2c1cc1n(c2=O)Cc2c-1nc1ccccc1c2CCNC(C)C]

The structure of rubitecan is shown below as Formula (X):

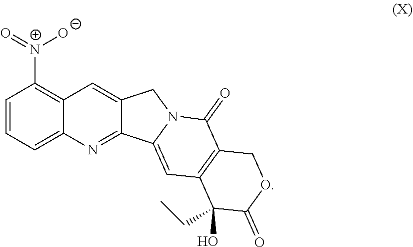 [SMILES: CC[C@@]1(O)C(=O)OCc2c1cc1n(c2=O)Cc2cc3c([N+](=O)[O-])cccc3nc2-1]

In particular, the present application is directed to methods and compositions employing irinotecan and topotecan, as well as analogs and derivatives thereof. These agents are both topoisomerase I inhibitors that are derivatives of camptothecin. Unless specifically excluded, analogs and derivatives of irinotecan or topotecan, including the compounds disclosed above, are considered to be within the scope of the invention.

The structure of irinotecan is shown below as Formula (XI):

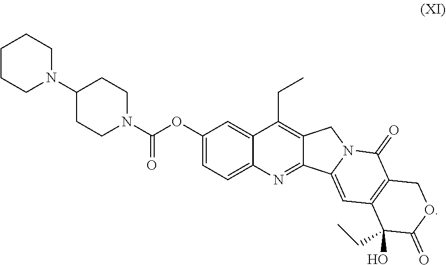 [SMILES: CCc1c2c(nc3ccc(OC(=O)N4CCC(N5CCCCC5)CC4)cc13)-c1cc3c(c(=O)n1C2)COC(=O)[C@]3(O)CC]

Irinotecan itself is activated in vivo by hydrolysis to SN-38, the active metabolite of irinotecan. Thus, irinotecan can be considered to be a prodrug. The structure of SN-38 is shown below as Formula (XII):

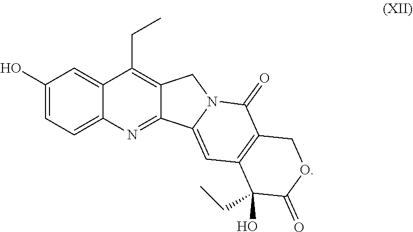 [SMILES: CCc1c2c(nc3ccc(O)cc13)-c1cc3c(c(=O)n1C2)COC(=O)[C@]3(O)CC]

Typically, irinotecan has been administered by 30-minute or 90-minute intravenous infusion, at either 125 mg/m² weekly for four of every six weeks or 350 mg/m² every three weeks. Alternative dosages, routes of administration, frequencies of administration, and durations of administration for irinotecan and its derivatives or analogs are provided below.

Irinotecan is a hydrophilic compound with a large volume of distribution (400 L/m²). At physiological pH, both irinotecan and SN-38 are present in two pH-dependent equilibrium isoforms; a form including the lactone ring, which is the form that has antineoplastic activity, and a form in which the lactone ring is opened by hydrolysis to form a carboxylate moiety which is essentially inactive. In plasma, the majority of irinotecan and SN-38 is bound to human serum albumin, which stabilizes the active lactone forms of these agents. In blood, irinotecan and SN-38 are largely bound to platelets and erythrocytes. Irinotecan has essentially linear pharmacokinetics; population pharmacokinetic models have assumed a three-compartmental model for irinotecan and a two-compartmental model for SN-38. The active metabolite SN-38 has a short distribution half-life (about 8 minutes). SN-38 reaches its peak plasma concentration within two hours after infusion. SN-38 also exhibits a second plasma concentration peak because of its enterohepatic recirculation and its release from erythrocytes. About 2-5% of irinotecan is hydrolyzed to SN-38 in the liver by two carboxylesterase converting enzymes (CES1 and CES2) and also in plasma by butyrylcholinesterase; CES2 has a 12.5-fold higher affinity for irinotecan than does CES1. After conversion, SN-38 is actively transported to the liver by the organic anion transporting polypeptide (OATP) 1B1 transporter.

SN-38 is then inactivated by glucuronidation to SN-38G (β-glucuronide conjugate) by several uridine diphosphate glucuronosyltransferase enzymes (UGTs) in the liver (UGT1A1, UGT1A9) and extrahepatic enzymes (UGT1A1, UGT1A7, UGT1A10) and excreted into the bile. Several UGT polymorphisms affects irinotecan pharmacokinetics, for example, the decreased UGT1 activity, may lead to severe toxicity. Also, UGT1A1 conjugates bilirubin and bilirubin glucuronidation is another risk factor for increased toxicity. The effect of these polymorphisms and their relevance for determining factors associated with the administration of irinotecan is addressed below. Additionally, intestinal bacteria produce β-glucuronidases that deconjugate SN-38G back to SN-38, resulting in enterohepatic recirculation of SN-38.

Irinotecan is metabolized by intrahepatic cytochrome P450 enzymes CYP3A4 and CYP3A5 into inactive metabolites APC (7-ethyl-10-[4-N-(5-aminopentanoic acid)-1-piperidino]carbonyloxycamptothecin) and NPC (7-ethyl-10-[4-amino-1-piperidino]carbonyloxycamptothecin). NPC can be further converted by CES1 and CES2 in the liver to SN-38.

Additionally, with respect to the metabolism of irinotecan, irinotecan is transported to bile by the ATP-binding cassette (ABC) transporter proteins, ABCB1, ABCC1, ABCC2, and ABCG2. Irinotecan clearance is mainly biliary, and estimated at a rate of 12-21 L/h/m². All metabolites, except SN-38G, are mainly excreted in feces. Irinotecan elimination half-life has been reported as being between 5-18 hr. SN-38 elimination half-life has been reported as being between 6-32 hours. There is a high inter-individual variability in irinotecan pharmacokinetic parameters which can be altered by several factors including age, sex, dose, timing of administration of irinotecan, hepatic function, enzyme activity, or hematocrit levels.

One aspect of the response to irinotecan involves genotypic variability; in particular, individuals with variants of the UGT1A1 gene called TA₇, which variant is also known as the “28 variant,” have reduced UGT1A1 expression in their liver. During chemotherapy, such individuals effectively receive a larger than expected dose because of slower clearance of the irinotecan. This can correspond to higher incidence of severe neutropenia and diarrhea in such individuals. It is now generally recommended that individuals that are homozygous for this polymorphism (the 28 variant) be administered lower dosages of irinotecan. Other genetic factors that can affect the optimum dose of irinotecan and the occurrence of significant side effects are addressed below.

U.S. Pat. No. 4,399,276 to Miyazawa et al. discloses derivatives or analogs of irinotecan including 7-substituted camptothecin derivatives of Formula (XIII):

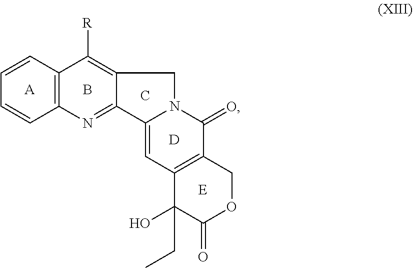

wherein:
(1) R is —CHO, —CH₂OR′, —CH(OR′)₂, or —CH═N—X;
(2) R′ is C₁-C₆ lower alkyl, phenyl(C₁-C₃) alkyl;
(3) X is hydroxyl or —NR¹R², where R¹ and R² are the same or different and where each is hydrogen or C₁-C₆ lower alkyl or, when R¹ is hydrogen, R² may be C₁-C₆ lower alkyl, a substituted or unsubstituted aryl group, a carbamoyl group, an acyl group, an aminoalkyl group, or an amidino group, or where R¹ is a lower alkyl group, R² may be an aminoalkyl group, or R¹ and R² may be combined together with the nitrogen atom to form a heterocyclic group. The compounds described in the reference include camptothecin-7-aldehyde, camptothecin-7-aldehyde oxime, camptothecin-7-aldehyde hydrazone, camptothecin-7-aldehyde hydrazone, camptothecin-7-aldehyde p-toluenesulfonylhydrazone, camptothecin-7-CH═N—N═C(NH₂)₂, camptothecin-7-CH═N—NH—COCH₂—N(CH₃)₂·HCl, camptothecin-7-CH═N—NH—COCH₂—N(CH₃)₃·Cl, camptothecin 7-aldehyde semicarbazone, camptothecin 7-aldehyde phenylsemicarbazone, camptothecin 7-aldehyde thiosemicarbazone, and camptothecin derivatives of Formulas (C-I), (C-II), (C-III), (C-IV), and (C-V):

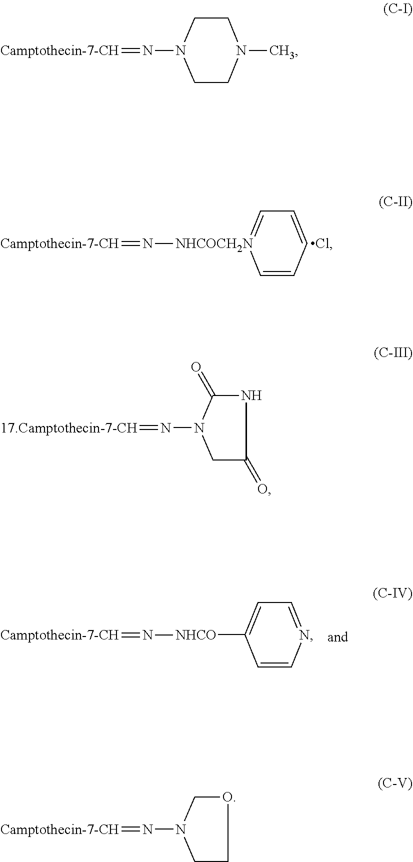

U.S. Pat. No. 4,399,282 to Miyazawa et al. discloses camptothecin derivatives of Formula (XIV):

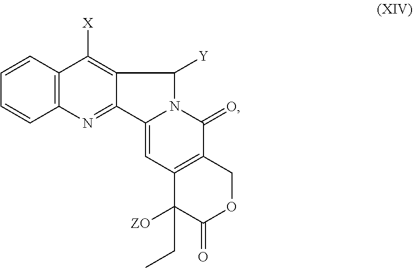

wherein:
(1) X is hydrogen, CH₂OH, carboxyl, alkyl, aralkyl, CH₂OR¹, or CH₂OR²;
(2) R¹ is an alkyl group or an acyl group;
(3) R² is a lower alkyl group;
(4) Y is hydrogen, hydroxyl, or OR³, wherein R³ is a lower alkyl group or an acyl group;
(5) Z is hydrogen or an acyl group;
   with the proviso that when X is CH₂OH, an alkyl group or an aralkyl group, both Y and Z are H; that when X is CH₂OR¹ or CH₂OR², Y is H; that when Y is hydroxyl, both X and Z are H; and that when Y is OR³, X is H. Among the camptothecin derivatives specifically disclosed in the reference are 7-hydroxymethylcamptothecin, 5-hydroxycamptothecin, 20-O-acetyl-7-acetoxymethylcamptothecin, 7-acetoxymethylcamptothecin, 7-succinoyloxymethylcamptothecin, 20-O-trifluoroacetyl-7-trifluoroacetoxymethylcamptothecin, 7-benzoyloxymethylcamptothecin, 7-propionyloxymethylcamptothecin, 7-butyryloxymethylcamptothecin, 7-caprylyloxymethylcamptothecin, 7-capryloxymethylcamptothecin, 7-isovaleryloxymethylcamptothecin, 7-phenylacetoxymethylcamptothecin, camptothecin-7-carboxylic acid, ethyl camptothecin-7-carboxylate, 5-methoxycamptothecin, 5-butoxycamptothecin, 5-acetoxycamptothecin, 20-O-acetyl-5-acetoxycamptothecin, 5-benzoyloxycamptothecin, 7-methylcamptothecin, 7-ethylcamptothecin, 7-propylcamptothecin, 7-butylcamptothecin, 7-heptylcamptothecin, 7-nonylcamptothecin, 7-isobutylcamptothecin, 7-benzylcamptothecin, 7-β-phenethylcamptothecin, 7-isopropylcamptothecin and 7-cyclohexylcamptothecin. The camptothecins can be derivatives of not only the naturally-occurring (+)-camptothecin, but also the (−)-camptothecin and the dl-camptothecin.

U.S. Pat. No. 4,604,463 to Miyazawa et al. discloses various camptothecin derivatives and methods for producing the camptothecin derivatives. Camptothecin itself is characterized by a pentacyclic structure consisting of quinoline (rings A and B), pyrroline (ring C), α-pyridone (ring D), and a six-membered lactone (ring E), as described above. The camptothecin derivatives are of Formula (XV):

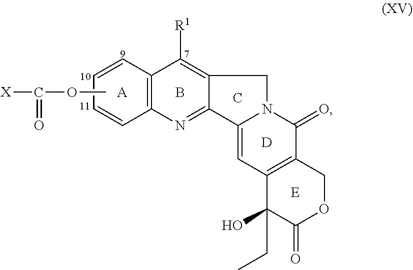

wherein R₁ is hydrogen, halogen, or C₁-C₄ alkyl; X is chlorine or —NR²R³ where R² and R³ are the same or different and each of R² and R³ is hydrogen or a substituted or unsubstituted C₁-C₄ alkyl or a substituted or unsubstituted carbocyclic or heterocyclic group, with the proviso that when both R² and R³ are substituted or unsubstituted alkyl groups, they may be combined together with the nitrogen atom to which R² and R³ are bonded to form a heterocyclic ring which may be interrupted with —O—, —S—, and/or >N—R⁴ in which R⁴ is hydrogen, a substituted or unsubstituted C₁-C₄ alkyl or a substituted phenyl group, and wherein the grouping —O—CO—X is bonded to a carbon atom located in any of the 9-, 10-, or 11-positions in the A ring of the camptothecin moiety. Suitable camptothecin derivatives include 9-chlorocarbonyloxycamptothecin (9-chlorocarbonyloxy-CPT; “camptothecin” will be referred to hereinafter simply as “CPT” in the derivatives); 9-chlorocarbonyloxy-7-ethyl-CPT; 10-chlorocarbonyloxy-CPT; 10-chlorocarbonyloxy-7-ethyl-CPT; 11-chlorocarbonyloxy-CPT; 11-chlorocarbonyloxy-7-ethyl-CPT; 7-ethyl-9-[4-(N-isopropylcarbamoylmethyl)-1-piperazino]carbonyloxy-CPT; 9-(1-piperazino)carbonyloxy-CPT; 9-(4-methyl-1-piperazino)carbonyloxy-CPT; 9-[4-(N-isopropylcarbamoylmethyl)-1-piperazino]carbonyloxy-CPT; 9-[4-(1-piperidino)-1-piperidino]carbonyloxy-CPT; 9-[N-methyl-N-(2-dimethylaminoethyl)]carbonyloxy-CPT; 7-ethyl-9-(1-piperazino)carbonyloxy-CPT; 7-ethyl-9-(4-methyl-1-piperazino)carbonyloxy-CPT; 7-ethyl-9-[4-(N-isopropylcarbamoylmethyl)-1-piperazino]carbonyloxy-CPT; 7-ethyl-9[4-(1-piperidino)-1-piperidino]carbonyloxy-CPT; 7-ethyl-9-[N-propyl-N-(2-dimethylaminoethyl)]carbonyloxy-CPT; 9-(1-piperazino)carbonyloxy-7-propyl-CPT; 10-[(N-ethoxycarbonylmethylamino)carbonyloxy]-7-ethyl-CPT; 10-(2-diethylamino)-ethyl-aminocarbonyloxy-7-ethyl-CPT; 10-diethylaminocarbonyloxy-7-ethyl-CPT; 7-ethyl-10-(4-morpholino)carbonyloxy-CPT; 7-ethyl-10-(1-piperazino)carbonyloxy-CPT; 7-ethyl-10-(4-methyl-1-piperazino)carbonyloxy-CPT; 7-ethyl-10-(4-ethyl-1-piperazino)carbonyloxy-CPT; 10-(4-benzyl-1-piperazino)carbonyloxy-7-ethyl-CPT; 7-ethyl-10-[4-(p-methoxyphenyl)-1-piperazino]carbonyloxy-CPT; 7-ethyl-10-[4-(3-hydroxypropyl)-1-piperazino]carbonyloxy-CPT; 7-ethyl-10-[4-(N-isopropylcarbamoylmethyl)-1-piperazino]carbonyloxy-CPT; 7-ethyl-10-[4-(1-piperidino)piperidino]carbonyloxy-CPT; 7-ethyl-10-[N-methyl-N-(2-dimethylaminoethyl)]aminocarbonyloxy-CPT; 7-ethyl-10-N-methyl-N-(1-methyl-4-piperidino)aminocarbonyloxy-CPT; 10-(4-morpholino)carbonyloxy-CPT; 10-(4-methyl-1-piperazino)carbonyloxy-CPT; 7-ethyl-10-(4-propyl-1-piperazino)carbonyloxy-CPT; 7-ethyl-10-(4-methyl-1-piperazino)carbonyloxy-CPT; 11-(4-ethyl-1-piperazino)carbonyloxy-CPT; 11-[4-(1-piperidino)-1-piperidino]carbonyloxy-CPT; 11-(1-piperazino)carbonyloxy-CPT; 11-(4-methyl-1-piperazino)carbonyloxy-CPT; 11-[4-(N-isopropylcarbamoylmethyl)-1-piperazino]carbonyloxy-CPT; 11-[N-methyl-N-(2-dimethylaminoethyl)]carbonyloxy-CPT; 7-ethyl-11-(1-piperazino)carbonyloxy-CPT; 7-ethyl-11-(4-methyl-1-piperazino)carbonyloxy-CPT; 7-ethyl-11-[4-(N-isopropylcarbamoylmethyl)-1-piperazino]carbonyloxy-CPT; 7-ethyl-11-[N-methyl-N-(2-dimethylaminoethyl)]carbonyloxy-CPT; and 7-ethyl-11-[4-(1-piperidino)-1-piperidino]carbonyloxy-CPT.

U.S. Pat. No. 5,955,466 to Ulrich discloses a method for preventing or decreasing diarrhea associated with irinotecan administration comprising the administration of tamoxifen at least two cell cycles prior to irinotecan administration. The major dose-limiting toxicity for the administration of irinotecan in cancer patients is a severe diarrhea which is delayed. Irinotecan was shown to induce a cell cycle block in S/G2 in cells of the intestinal tract. Other possible remedies or prophylactic agents include loperamide, baicalin, antibiotics, or octreotide. Some of these agents can act by reducing beta-glucuronidase activity; that enzyme is responsible for the deconjugation of the glucuronide form of the active irinotecan metabolite, SN-38.

U.S. Pat. No. 6,087,377 to Ulrich discloses a method for preventing or decreasing diarrhea associated with irinotecan administration comprising the administration of an antiestrogen at least two cell cycles prior to irinotecan administration. The antiestrogen can be droloxifene, TAT-59, or raloxifene.

U.S. Pat. No. 6,881,420 to Flashner-Barak et al. is directed to oral dosage forms and compositions for administration of irinotecan (and other agents), whose oral effectiveness is limited by pre-systemic and systemic deactivation in the gastrointestinal tract. Irinotecan has increased bioavailability if delivered to the stomach without increased side effects. Gastric release of irinotecan delivers it to the acidic environment of the stomach, which is advantageous for minimizing ring-opening of the lactone form of the drug to the inactive hydroxyacid form. A greater proportion of the irinotecan is thus presented to the carboxylesterase enzymes in the gastrointestinal tract in active form. This results in a greater production of SN-38. In particular, a solid pharmaceutical dosage form is disclosed for enhanced systemic delivery of irinotecan comprising irinotecan and a gastric retention vehicle composition comprising a hydrogel, wherein the dosage form expands upon contact with gastric fluid and wherein after ingestion by a patient the gastric retention vehicle composition expands to retain the dosage form in the patient's stomach for a period of three hours or more. The dosage forms can contain a unit dose of from about 20 to about 250 milligrams of irinotecan. The dosage forms can further comprise tannic acid. The dosage forms can further comprise a superdisintegrant, which can be selected from the group consisting of crospovidone, croscarmellose sodium, sodium starch glycolate and mixtures thereof. The hydrogel can be selected from the group consisting of hydroxypropyl methylcellulose and mixtures of hydroxypropyl methylcellulose and hydroxypropylcellulose. In one alternative, the gastric retention vehicle composition comprises: (i) from about 20 to about 70 weight percent of the hydrogel, the hydrogel comprising hydroxypropyl methylcellulose and hydroxypropylcellulose in a weight ratio of from about 1:3 to about 5:3; (ii) from about 25 to about 75 weight percent of the superdisintegrant; and (iii) from about 2 to about 10 weight percent tannic acid.

U.S. Pat. No. 7,122,553 to Rahman et al. discloses liposomal formulations of irinotecan. Typically, the liposomal formulation comprises a first liposome forming material comprising cardiolipin and a second liposome forming material and wherein the composition comprises about 1 weight percent to about 50 weight percent irinotecan, about 1 weight percent to about 50 weight percent cardiolipin, about 1 weight percent to about 95 weight percent phosphatidylcholine, and about 0.001 weight percent to about 5 weight percent α-tocopherol.

U.S. Pat. No. 7,435,818 to Chen et al. discloses four specific crystalline forms of irinotecan hydrochloride (polymorphs) and crystallization methods for preparation of these polymorphic forms.

U.S. Pat. No. 7,479,499 to Govindarajan et al. discloses compositions comprising thalidomide and irinotecan for the treatment of colorectal cancer. Irinotecan contains a chiral center, and can be used as a racemate, as an optically pure compound, or as a preparation that is enriched in one enantiomer. Methods for treatment of colorectal cancer involving either simultaneous or sequential administration of thalidomide and irinotecan are described.

U.S. Pat. No. 7,488,825 to Shimizu et al. discloses further polymorphisms of irinotecan hydrochloride and methods for their preparation.

U.S. Pat. No. 7,683,170 to Wissmann et al. discloses methods for the preparation of irinotecan.

U.S. Pat. No. 7,763,438 to Muraca discloses gene and protein expression profiles and methods of using them in colorectal cancer patients that can predict response to irinotecan. Specifically, results of gene expression analysis showed that in colon cancer patients who were responsive to treatment with irinotecan, the following genes were up-regulated: ERBB2, GRB7, JNK1 kinase, BCL2, MK167, phospho-Akt, CD-68 and BAG1, and the following genes were down-regulated: Erk1 kinase, phospho-GSK-3β, MMP11, CTSL2, CCNB1, BIRC5, STK6, MRP14 and GSTM1, compared with expression of these genes in the normal colon tissue samples from these patients, and from the negative control patients, i.e., the tissue samples from patients that had experienced a recurrence of their cancer after treatment with irinotecan. Reference genes ACTB, GAPD, GUSB, RPLP0 and TFRC all were up-regulated. For protein expression, in colon cancer patients who were responsive to treatment with irinotecan, the following proteins were up-regulated: ERBB2, GRB7, JNK1 kinase, BCL2, MK167, phospho-Akt, CD-68 and BAG1, and the following proteins were down-regulated: Erk1 kinase, phospho-GSK-3β, MMP11, CTSL2, CCNB1, BIRC5, STK6, MRP14 and GSTM1, compared with expression of these proteins in the normal colon tissue samples from these patients, and in the negative control samples, i.e., colon tumor samples from patients that had experienced a recurrence of their cancer after treatment with irinotecan (non-responders). Additionally, IHC analysis showed that a majority of these proteins were not up- or down-regulated in the positive control tissue samples. The reference proteins ACTB, GAPD, GUSB, RPLP0 and TFRC all were up-regulated. This could be used in a method of administering irinotecan or analogs to provide greater therapeutic efficacy, or possibly to adjust the dosage to reduce the dosage where the genetic or protein profile indicates that the patient is more likely to respond, thereby reducing the likelihood of significant side effects.

U.S. Pat. No. 7,807,350 to Ratain et al. is generally directed to determining the likelihood of irinotecan toxicity based on the genotype at position −3156 of the UGT1A1 gene or at any position in linkage disequilibrium with the −3156 variant. Irinotecan hydrolysis by carboxylesterase-2 is responsible for its activation to SN-38, a topoisomerase I inhibitor of much higher potency than irinotecan. The main inactivating pathway of irinotecan is the biotransformation of active SN-38 into inactive SN-38 glucuronide (SN-38G). Interpatient differences in systemic formation of SN-38G have been shown to have clear clinical consequences in patients treated with irinotecan. Patients with higher glucuronidation of SN-38 are more likely to be protected from the dose-limiting toxicity of diarrhea when irinotecan is administered on a weekly schedule. SN-38 is glucuronidated by UDP-glucuronosyltransferase 1A1 (UGT1A1). The nucleotide at position −3156 in the UGT1A1 is correlated with irinotecan toxicity. An A at that position positively correlates with irinotecan toxicity while a G at that position correlates with tolerance to irinotecan (less toxicity). If the subject is homozygous for A (A in both alleles of the subject's genome), the risk of toxicity increases.

U.S. Pat. No. 7,846,473 to Yoshino et al. discloses formulations of irinotecan employing a liposome. Typically, the formulation comprises a liposome formed by a membrane of a lipid bilayer containing a phospholipid as a membrane component, wherein only the outer surface of the liposome is modified with a surface-modifying agent containing a polyethylene glycol, in which irinotecan and/or a salt thereof is encapsulated at a concentration of at least 0.1 mol/mol (drug mol/membrane total lipid mol) by an ion gradient between an inner aqueous phase and an outer aqueous phase of the liposome.

U.S. Pat. No. 7,897,772 to Shimizu et al. discloses an acid addition salt of irinotecan which is formed through addition of an acid selected from the group consisting of sulfuric acid, nitric acid, phosphoric acid, methanesulfonic acid, citric acid, maleic acid, and succinic acid, processes for preparing such acid addition salts, and pharmaceutical compositions including the acid addition salts. According to the reference, a pharmaceutical composition containing the irinotecan acid addition salt and a pharmaceutically acceptable carrier is useful as an injection aqueous product, a peroral drug product, and other drug products. In the case where an aqueous drug product is prepared, examples of the pharmaceutically acceptable carrier employed include purified water, physiological saline, a pH-modifier, a tonicity agent, a stabilizer, and a buffer. In the case where a peroral drug product is prepared, examples of the pharmaceutically acceptable carrier include an excipient, a lubricant, a binder, a disintegrant, a colorant, a taste-controlling agent, and a flavoring agent. The peroral product may be in the form of, for example, a tablet, granules, a powder, or a capsule.

U.S. Pat. No. 7,943,311 to Okamura et al. discloses a method for determining the risk of adverse effects of irinotecan by detecting polymorphisms in the TATA box within the promoter region of the UDP glucuronosyl transferase gene. Polymorphisms that predispose to serious side effects associated with the administration of irinotecan have 7 TA repeats in the TATA box within the promoter region instead of 6 TA repeats in the wild-type promoter. This lowers the gene expression of UGT1A1 and results in lower UDP glucuronosyl transferase activity. Probes for detecting such polymorphisms and kits including such probes are disclosed.

U.S. Pat. No. 8,147,867 by Hong et al. discloses liposomes for the delivery of a number of therapeutic agents, including camptothecins such as irinotecan or topotecan. The interior of the liposome contains a substituted ammonium moiety of Formula (A-I):

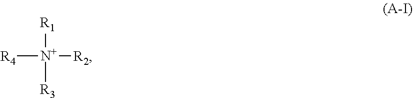

wherein each of R₁, R₂, R₃, and R₄ is independently a hydrogen or an organic group having, inclusively, in totality up to 18 carbon atoms, wherein at least one of R₁, R₂, R₃, and R₄ is an organic group, wherein the organic group is independently a hydrocarbon group having up to 8 carbon atoms, and is an alkyl, alkylidene, heterocyclic alkyl, cycloalkyl, aryl, alkenyl, or cycloalkenyl group or a hydroxy-substituted derivative thereof, optionally including within its hydrocarbon chain a S, O, or N atoms, forming an ether, ester, thioether, amine, or amide bond, wherein at least three of R₁, R₂, R₃, and R₄ are organic groups, or the substituted ammonium is a sterically hindered ammonium, such as, for example, where at least one of the organic groups has a secondary or tertiary carbon atom directly linked to the ammonium nitrogen atom. Preferably, the substituted ammonium compound encapsulated into liposomes has a negative logarithm of the acidic (deprotonation) dissociation constant (pKa) of at least about 8.0, at least about 8.5, at least about 9.0, at least 9.5, or at least 10.0, as determined in an aqueous solution at ambient temperature. The liposomes can also contain a polyanion. wherein the polyanion is a polyanionized polyol or a polyanionized sugar. Suitable substituted ammonium compounds include isopropylethylammonium, isopropylmethylammonium, diisopropylammonium, t-butylethylammonium, dicychohexylammonium, protonized forms of morpholine, pyridine, piperidine, pyrrolidine, piperazine, t-butylamine, 2-amino-2-methylpropanol-1,2-amino-2-methyl-propandiol-1,3, tris-(hydroxyethyl)-aminomethane, trimethylammonium, triethylammonium, tributyl ammonium, diethylmethylammonium, diisopropylethyl ammonium, triisopropylammonium, N-methylmorpholinium, N-hydroxyethylpiperidinium, N-methylpyrrolidinium, N,N′-dimethylpiperazinium, tetramethylammonium, tetraethylammonium, and tetrabutylammonium. The membrane of the liposome can constitute a polymer-conjugated ligand. Typically, for these liposomes, when administered into the bloodstream of a mammal, the irinotecan has a half-release time from the liposomes of at least 24 hours and the irinotecan entrapped inside the liposomes is at a concentration that exceeds the irinotecan concentration in the aqueous medium.

U.S. Pat. No. 8,247,426 to Pozzi et al. discloses a crystalline polymorphic form of irinotecan.

U.S. Pat. No. 9,107,918 to Nishiyama et al. discloses that expression of the following genes: AMD1, CTSC, EIF1AX, C12orf30, DDX54, PTPN2, and TBX3 can affect the therapeutic efficacy of irinotecan. Furthermore, the following factors have been reported relating to the sensitivity or resistance of irinotecan: mutation of topoisomerase I, which is a target of SN-38, and expression level thereof; activity of carboxylesterase, the enzyme involved in conversion of CPT-11 to SN-38; ABC transporter genes (multidrug resistance protein (MRP)-1, MRP-2, and breast cancer resistant protein (BCRP)/ABCG2), which affects the intracellular accumulation amounts of CPT-11 and SN-38; and BCL2 family genes. Studies have been conducted on correlations of cell proliferation antigen Ki-67, tumor suppressor gene TP53, and other genes or proteins with response to CPT-11 therapy. Recently, a clinical study has revealed that the plasma level of tissue inhibitor of metalloproteinase-1 (TIMP-1), the TIMP-1 having anti-apoptosis action, is significantly correlated with the clinical prognosis of a metastatic colorectal cancer patient having undergone CPT-11+5-FU combination therapy. However, a study has revealed that neither topoisomerase I (target) nor thymidylate synthase (possible 5-FU-sensitivity predicting factor) has a clear correlation with therapeutic response in 5-FU+CPT-11 combination therapy.

U.S. Pat. No. 9,339,497 to Bayever et al. discloses methods for treating pancreatic cancer by administering liposomal irinotecan (MM-398) alone or in combination with additional therapeutic agents. In one embodiment, the liposomal irinotecan (MM-398) is co-administered with 5-fluorouracil and leucovorin. MM-398 is a nanoliposomal formulation of irinotecan (irinotecan sucrose sulfate liposome injection). An MM-398 liposome is a unilamellar lipid bilayer vesicle of approximately 80-140 nm in diameter that encapsulates an aqueous space which contains irinotecan complexed in a gelated or precipitated state as a salt with sucrose octasulfate. The lipid membrane of the liposome is composed of phosphatidylcholine, cholesterol, and a polyethyleneglycol-derivatized phosphatidyl-ethanolamine in the amount of approximately one polyethyleneglycol (PEG) molecule for 200 phospholipid molecules. In general, the method comprises a method of treating metastatic adenocarcinoma of the pancreas in a human patient who has previously been treated with the antineoplastic agent gemcitabine, the method comprising intravenously administering to the patient once every two weeks 80 mg/m² of the antineoplastic agent MM-398 liposomal irinotecan in combination with 200 mg/m² of (I)-form of leucovorin or 400 mg/m² of the (I+d) racemic form of leucovorin and 2400 mg/m² of the antineoplastic agent 5-fluorouracil to treat the metastatic adenocarcinoma of the pancreas in the human patient, where no other antineoplastic agent is administered to the human patient for treatment of the metastatic adenocarcinoma of the pancreas. The patient can be premedicated with dexamethasone and a 5-HT3 antagonist or other anti-emetic.

U.S. Pat. No. 9,364,473 to Bayever et al. is also directed to methods of treating pancreatic cancer using liposomal irinotecan. The patient can be homozygous for the UGT1A1*28 allele) with 7 TA repeats; these patients exhibit reduced glucuronidation of SN-38 and may be at increased risk of side effects from administration of irinotecan.

U.S. Pat. No. 9,452,162 to Bayever et al. is also directed to methods of treating pancreatic cancer using liposomal irinotecan.

U.S. Pat. No. 9,492,442 to Bayever et al. is also directed to methods of treating pancreatic cancer using liposomal irinotecan. The liposomal irinotecan can be administered in 500 mL of a 5% dextrose solution.

U.S. Pat. No. 9,616,081 to Okabe is directed to a combination therapy involving administering to a subject a combination drug comprising trifluridine and tipiracil hydrochloride in a molar ratio of 1:0.5 at a dose of 35 to 70 mg/m²/day of trifluridine, and 45 to 144 mg/m²/day of irinotecan hydrochloride hydrate. The combination therapy can be used to treat colorectal cancer, lung cancer, breast cancer, pancreatic cancer, or gastric cancer.

U.S. Pat. No. 9,765,083 to Zabudkin et al. discloses a method for the synthesis of 7-ethyl-10-[4-(1-piperidino)-1-piperidino]carbonyloxycamptothecin (i.e. irinotecan), comprising: (a) preparing 10-[4-(1-piperidino)-1-piperidino]carbonyloxycamptotecin; and (b) selectively ethylating the compound of step (a) at the 7-position, thus resulting in the preparation of 7-ethyl-10-[4-(1-piperidino)-1-piperidino]carbonyloxycamptothecin. The invention described in the reference is further directed to the use of 10-[4-(1-piperidino)-1-piperidino]carbonyloxycamptothecin (i.e., 7-des-ethyl-irinotecan) as intermediate in a method for the synthesis of irinotecan as described.

U.S. Pat. No. 10,022,365 to Tong et al. discloses liposomes of irinotecan or irinotecan hydrochloride and methods for the preparation of the liposome. The liposome contains irinotecan or irinotecan hydrochloride, neutral phospholipid and cholesterol, wherein the weight ratio of the cholesterol to the neutral phospholipid is 1:3 to 1:5. The liposome is prepared by an ion gradient method. In one alternative, the liposome comprises irinotecan hydrochloride, hydrogenated soybean phosphatidylcholine, polyethylene glycol 2000-distearoyl phosphatidyl ethanolamine, cholesterol, and ethylenediaminetetraacetic acid disodium, wherein the weight ratio of the cholesterol to the hydrogenated soybean phosphatidylcholine is about 1:4, and there is no significant change in the particle size and encapsulation efficiency of the liposome after the liposome is stored at 25° C. for 60 days.

U.S. Pat. No. 10,143,657 to Hojgaard discloses a solid pharmaceutical composition comprising irinotecan as a free base or hydrochloride and a mixture comprising a vehicle and a non-ionic surfactant in an amount sufficient to achieve solubilization of the irinotecan. Typically, the composition is coated with an enteric coating. In one alternative, the irinotecan is solubilized in a mixture comprising (a) a vehicle, wherein the vehicle is selected from a saturated or unsaturated fatty acid of between 8-24 carbon atoms in length and a polyethylene glycol, having an average molecular weight of at least 3000 and (b) a water soluble non-ionic surfactant, wherein the water-soluble surfactant is selected from poloxamers, a tocopherol polyethylene glycol succinate derivative, lauroyl polyoxylglycerides, polysorbate 80, polyoxyl 40 hydrogenated castor oil, polyoxyl 35 castor oil, caprylocaproyl macrogolglycerides, polyoxyl 15 hydroxystearate and polyoxyethylene 10 oleyl ether, wherein the irinotecan is in a solid core comprising about 0.5% to about 30% by weight of the irinotecan. Pharmaceutical compositions can contain further excipients such as fillers, diluents, binders, lubricants, glidants, enhancers, wetting agents, surfactants, antioxidants, metal scavengers, pH-adjusting agents, acidifying agents, alkalizing agents, preservatives, buffering agents, chelating agents, stabilizing agents, coloring agents, complexing agents, emulsifying and/or solubilizing agents, absorption enhancing agents, release modifying agents, flavoring agents, taste-masking agents, humectants, and sweetening agents.

U.S. Pat. No. 10,172,943 to Choi et al. discloses an irinotecan-loaded dual-reverse thermosensitive formulation, which is a dual-reverse thermosensitive hydrogel composition including nanoparticles including irinotecan and lipids; a hydrogel; and a stabilizer. In one alternative, the formulation comprises: (a) a thermosensitive nanoparticle comprising irinotecan as an active ingredient, and a lipid mixture comprising tricaprin and triethanolamine mixed at a weight ratio of 99.9:0.1 to 10:90; and (b) a thermosensitive hydrogel having a gelation temperature of 30 to 36° C., comprising poloxamer 188, poloxamer 407 or a mixture thereof, and Tween 80, wherein the lipid mixture has a melting point of 30 to 36° C.

U.S. Pat. No. 10,919,905 to Liao et al. discloses polymorphic forms for irinotecan free base. There are two polymorphic forms designated S1 and S2, with different X-ray powder diffraction patterns.

U.S. Pat. No. 11,033,606 to Castan discloses a pharmaceutical composition comprising aflibercept, folinic acid, 5-fluorouracil, and irinotecan (FOLFIRI) to treat colorectal cancer. Aflibercept is a fusion protein comprising the signal sequence of VEGFR1 fused to the D2 Ig domain of the VEGFR1 receptor, itself fused to the D3 Ig domain of the VEGFR2 receptor, in turn fused to the Fc domain of IgG1A.

U.S. Pat. No. 11,071,726 to Fitzgerald et al. discloses combination therapy methods for gastric cancer using liposomal irinotecan, oxaliplatin, 5-fluorouracil, and, optionally, leucovorin. The liposomal irinotecan comprises irinotecan sucrose octasulfate 1,2-distearoyl-sn-glycero-3-phosphocholine (DSPC), cholesterol, and a N-(carbonylmethoxypolyethlyene glycol-2000)-1,2-distearoyl-sn-glycero-3-phosphoethanolamine (MPEG-2000-DSPE).

U.S. Pat. No. 11,090,299 to Park et al. discloses an oral solid formulation including irinotecan or a pharmaceutically acceptable salt thereof and an acidifying agent. In one particularly preferred formulation, the oral solid formulation comprises wet granules comprising irinotecan hydrochloride as a sole active ingredient; 10 wt % to about 23 wt % of lactose and 45 wt % to about 57 wt % of microcrystalline cellulose based on a total weight of the oral solid formulation; and an acidifying agent in an amount of 0.2 parts to 5 parts by weight based on 1 part by weight of the irinotecan hydrochloride, wherein the acidifying agent is selected from the group consisting of acetic acid, citric acid, lactic acid, and a combination thereof, wherein a dissolution rate of the irinotecan hydrochloride of the oral solid formulation is about 80% or greater in initial 30 minutes. The pharmaceutically acceptable salt may include an inorganic acid salt or an organic acid salt. The inorganic acid salt can be a hydrochloride, a phosphate, a sulfate, or a disulfate. The organic acid salt can be a malate, maleate, citrate, fumarate, besylate, camsylate (camphorsulfonate), or edisylate (ethanedisulfonate). Suitable acidifying agents can include inorganic acids such as hydrochloric acid, phosphoric acid, potassium dihydrogen phosphate, sodium dihydrogen phosphate, or any combinations thereof. Suitable acidifying agents can also include organic acids such as citric acid, lactic acid, tartaric acid, fumaric acid, phthalic acid, acetic acid, oxalic acid, malonic acid, adipic acid, phytic acid, succinic acid, glutaric acid, maleic acid, malic acid, mandelic acid, ascorbic acid, benzoic acid, methanesulfonic acid, capric acid, caproic acid, caprylic acid, lauric acid, arachidic acid, erucic acid, linoleic acid, linolenic acid, oleic acid, palmitic acid, myristic acid, edysilic acid, stearic acid, or any combinations thereof, or, alternatively, a C₂-C₂₀ organic acid that is a carboxylic acid or a sulfonic acid. The oral solid formulation may be formulated as, but is not limited to, a pellet, a capsule, a tablet (including a single-layered tablet, a double-layered tablet, and a pressed core tablet), dry syrups or granules. The oral solid formulation may include pharmaceutically acceptable additives such as a diluent, a binder, a disintegrant, a lubricant, and any combinations thereof.

U.S. Pat. No. 11,123,326 to Stancato discloses a method of treating rhabdomyosarcoma that involves administering to the patient 5-(5-(2-(3-aminopropoxy)-6-methoxyphenyl)-1H-pyrazol-3-ylamino)pyrazine-2-carbonitrile or a pharmaceutically acceptable salt thereof, such as a formate, a dihydrochloride, or a methanesulfonate, and irinotecan. The 5-(5-(2-(3-aminopropoxy)-6-methoxyphenyl)-1H-pyrazol-3-ylamino)pyrazine-2-carbonitrile is a CHK1/CHK2 inhibitor.

United States Patent Application Publication No. 2002/0169141 by Martin et al. discloses a dosage form and a method of administering an anti-tumor composition comprising tegafur, uracil, and folinic acid to potentiate the coadministration of irinotecan. The tegafur and uracil produce 5-fluorouracil. The composition can be administered orally.

United States Patent Application Publication No. 2004/0266704 by Miller et al. discloses a method for treating locally advanced or metastatic breast cancer in a patient who demonstrated failure of prior treatment with an anthracycline, a taxane and a fluoropyrimidine, which comprises administering a therapeutically effective amount of irinotecan.

United States Patent Application Publication No. 2005/0019387 by Rahman et al. discloses therapeutic compositions including liposomal entrapped irinotecan wherein the liposome comprises cardiolipin and a second liposome-forming material that is a lipid selected from the group consisting of phosphatidyl choline, cholesterol, α-tocopherol, dipalmitoyl phosphatidyl choline and phosphatidyl serine.

United States Patent Application Publication No. 2005/0032724 by Heinrich et al. discloses method of using irinotecan to treat a patient suffering from cancer which comprises: (1) determining if the patient has one or more variant alleles of the MRP1 gene in the cancerous tissue; and (2) in a patient having one or more of such variant alleles, administering to the patient an amount of irinotecan which is sufficient to treat a patient having such variant alleles which amount is increased or decreased in comparison to the amount that is administered without regard to the patient's alleles in the MRP1 gene. The patients can also be treated with an MRP inhibitor, such as valspodar (SDZ-PSC 833), tert-butyl 2-[(3S,6S,9S,15S,21S,24S,27S,30S)-15,18-bis[(2S)-butan-2-yl]-6-[(4-methoxyphenyl)methyl]-3,10,16,19,22,28-hexamethyl-2,5,8,11,14,17,20,23,26,29-decaoxo-9,24,27-tri(propan-2-yl)-4-oxa-1,7,10,13,16,19,22,25,28-nonazabicyclo[28.4.0]tetratriacontan-21-yl]acetate (SDZ 280-446), sodium 3-[[3-[(E)-2-(7-chloroquinolin-2-yl)ethenyl]phenyl]-[3-(dimethylamino)-3-oxopropyl]sulfanylmethyl]sulfanylpropanoate (MK571), dofequidar (MS209), 2-(4-benzhydrylpiperazin-1-yl)ethyl 5-[(4R,6R)-4,6-dimethyl-2-oxo-1,3,2lambda5-dioxaphosphinan-2-yl]-2,6-dimethyl-4-(3-nitrophenyl)pyridine-3-carboxylate (PAK-104p), verapamil, benzbromarone, dipyridamole, furosemide, gamma-GS(naphthyl)cysteinyl-glycine diethyl ester, genistein, quinidine, rifampicin, mifepristone (RU-486), or sulfinpyrazone.

United States Patent Application Serial No. 2005/0272737 by Chen et al. discloses treatment of malignancies with irinotecan and a EGFR kinase inhibitor such as erlotinib, as well as a pharmaceutical composition that comprises irinotecan and an EGFR kinase inhibitor. Other EGFR kinase inhibitors such as lapatinib or gefitinib can alternatively be used.

United States Patent Application Publication No. 2006/0030578 by Ahmad et al. discloses a method for preparing liposomal irinotecan by first inactivating irinotecan prior to liposome formation and then subsequently activating the irinotecan by lowering the pH of the lipid composition to an acidic pH of less than about 3.5, such as between 1.5-3.0 or about 2. A protective sugar can be added. The lipid phase can comprise cardiolipin and at least one additional lipid component selected from the group consisting of phosphatidylcholine, phosphatidylethanolamine, phosphatidylserine, phosphatidylglycerol, phosphatidic acid, phosphatidylinositol, sphingomyelin, sterol, tocopherol, fatty acid, and mixtures thereof.

United States Patent Application Publication No. 2006/0046993 by Forino et al. discloses a crystalline polymorphic form of irinotecan hydrochloride and processes for its preparation. The crystalline polymorphic form is characterized by its X-ray powder diffraction pattern. A prior crystalline form of irinotecan hydrochloride dihydrate is described as “Form b.”

United States Patent Application Publication No. 2007/0208050 by Palle et al. discloses a process for preparing irinotecan or salts thereof. In general, the process comprises purifying 7-ethyl-10-hydroxycamptothecin by: i) slurrying 7-ethyl-10-hydroxycamptothecin in an alcohol; then ii) dissolving 7-ethyl-10-hydroxycamptothecin in acetic acid, removing acetic acid to form a concentrated solution, and combining with an alcohol to form a precipitate; then iii) recrystallizing 7-ethyl-10-hydroxycamptothecin.

United States Patent Application Publication 2008/0182990 by Vishnukant et al. discloses a process for the preparation of irinotecan hydrochloride trihydrate with enhanced yield, purity by contacting 1-chlorocarbonyl-4-piperidinopiperidine hydrochloride with 7-ethyl-10-hydroxy-camptothecin to obtain crude irinotecan which is subsequently purified by solvent treatment, obtaining purified irinotecan which is converted into irinotecan hydrochloride trihydrate.

United States Patent Application Publication No. 2009/0062323 by Czarnik discloses deuterium-enriched irinotecan and processes for its preparation.

United States Patent Application Publication No. 2010/0160233 by Bissery et al. discloses antitumor combinations of VEGF inhibitors with irinotecan. A particularly preferred VEGF inhibitor is a fusion protein comprising the signal sequence of VEGFR1 fused to the D2 Ig domain of the VEGFR1 receptor, itself fused to the D3 Ig domain of the VEGFR2 receptor, in turn fused to the Fc domain of IgG1, also known as VEGFR1 R2-FcΔC1 or Flt1 D2.Flk1 D3.FcΔC1.

United States Patent Application Publication No. 2010/0247533 by Friess et al. discloses treatment of malignancies with a humanized anti-EGFR IgG1 antibody and irinotecan. The humanized anti-EGFR IgG1 antibody includes oligosaccharides in the Fc region.

United States Patent Application Publication Nos. 2011/0136253 and 2011/0165699 by Salamone et al. disclose irinotecan immunoassays.

United States Patent Application Publication No. 2012/0282325 by Tong et al. discloses liposomes of irinotecan or irinotecan hydrochloride; the liposomes contain irinotecan or irinotecan hydrochloride, neutral phospholipid, and cholesterol, wherein the weight ratio of the cholesterol to the neutral lipid is 1:3 to 1:5. The liposome is prepared by an ion gradient method.

United States Patent Application Publication No. 2013/0274281 by Bradley discloses methods of treating metastatic breast cancer with 4-iodo-3-nitrobenzamide or a metabolite or salt thereof and irinotecan. Metabolites of 4-iodo-3-nitrobenzamide include 4-iodo-3-aminobenzoic acid and 4-iodo-3-aminobenzamide.

United States Patent Application Publication No. 2014/0349945 by Xu et al. discloses PEG-amino acid-oligopeptide-irinotecan drug conjugates of the formula:

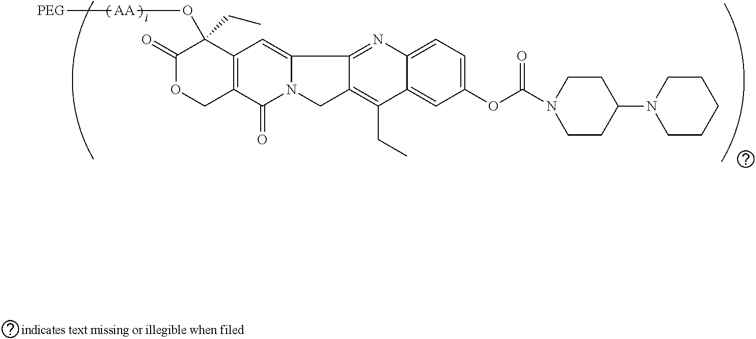

wherein:
(1) PEG is polyethylene glycol with a molecular weight of 300 to 60,000 daltons;
(2) (AA); represents an oligopeptide, wherein the amino acids comprising the oligopeptide can be the same or different;
(3) i and j can be the same or different, and i is an integer of 2-12 that is the number of amino acids in the oligopeptide, and j is an integer of 2-12 that is the number of irinotecan moieties linked with the oligopeptide. The PEG can be straight-chain or branched-chain. Typically, the oligopeptide includes glutamic acid and glycine.

United States Patent Application Publication No. 2017/0087146 by Li et al. discloses an irinotecan hydrochloride composite phospholipid composition comprising irinotecan hydrochloride, composite phospholipid, cholesterol, long-circulating membrane material, surfactant and a buffer medium. The composite phospholipid consists of hydrogenated soybean phospholipids and other lipids; the other lipids can be one or more lipids selected from the group consisting of soybean phospholipid, egg phosphatidylcholine, hydrogenated egg phosphatidylcholine, sphingomyelin, cardiolipin, distearoyl phosphatidylcholine, dipalmitoyl phosphatidylcholine, dimyristoyl phosphatidylcholine, dioleoyl phosphatidylcholine, distearoyl phosphatidylethanolamine, dipalmitoyl phosphatidylethanolamine, dimyristoyl phosphatidylethanolamine, dioleoyl phosphatidylethanolamine, distearoyl phosphatidylglycerol, dipalmitoyl phosphatidylglycerol, dimyristoyl phosphatidylglycerol, and dioleoyl phosphatidylglycerol. The long circulating membrane material can be polyethylene glycol derivatized phospholipids formed by covalently binding polyethylene glycol molecules with reactive groups on phospholipid molecules, which can be selected from the group consisting of polyethylene glycol derivatized phospholipids selected from polyethylene glycol-phosphatidylethanolamine, polyethylene glycol-dimyristoyl phosphatidylethanolamine, polyethylene alcohol-dipalmitoyl phosphatidyl ethanolamine, and polyethylene glycol-distearoyl phosphatidylethanolamine (PEG-DSPE). The nonionic surfactant can be selected from the group consisting of Pluronic F68, Pluronic F127, Pluronic P123, Pluronic P85, Pluronic L61, TPGS and HS15. The buffer can be selected from the group consisting of histidine buffer, glycine buffer, phosphate buffer and 4-hydroxyethyl piperazine-ethanesulfonic acid (HEPES) buffer.

United States Patent Application Publication No. 2017/0333421 by Adiwijaya et al. discloses the population pharmacokinetics of a preparation of liposomal formulation of irinotecan designated NaI—IRI with a longer half-life (t_{1/2}), higher plasma total irinotecan (tIRI), and lower SN-38 maximum concentration (Cₘₐₓ) compared with non-liposomal irinotecan.

United States Patent Application Publication No. 2018/0110771 by Drummond et al. discloses a liposomal preparation of irinotecan, in particular a storage stabilized liposomal irinotecan composition comprising irinotecan sucrose octasulfate (SOS) encapsulated in irinotecan liposomes comprising one or more phospholipids with a ratio corresponding to a total of 500 grams irinotecan moiety (±10% by weight) per mol total phospholipids, the liposomal irinotecan composition stabilized to have less than 20 mol % (with respect to total phospholipids) lyso-PC during the first 6 months of storage of the liposomal irinotecan composition at about 4° C., the liposomal irinotecan composition obtained by a process comprising the steps of: (a) forming liposomes from triethylamine sucrose octasulfate and/or diethylamine sucrose octasulfate having a total sulfate concentration from 0.4 to 0.5 M, cholesterol and phospholipids comprising DSPC and a compound comprising polyethylene glycol and distearoylphosphatidyl ethanolamine; (b) contacting the liposomes with a solution comprising the irinotecan moiety and made using irinotecan free base or irinotecan salt at a temperature above the transition temperature of the phospholipids in the liposomes, thereby forming a preparation of irinotecan liposomes encapsulating the irinotecan moiety in the irinotecan sucrose octasulfate in the liposomes to form the irinotecan liposomes; and (c) adjusting the pH of the preparation of irinotecan liposomes to be from 7.0 to 7.5.

United States Patent Application Publication No. 2018/0237833 by Oka et al. discloses a method for predicting a risk of occurrence of a side effect of irinotecan by analyzing a single nucleotide polymorphism in a region encoding a specific gene. The prediction of the risk of the occurrence of a side effect of irinotecan is assisted by analyzing a single nucleotide polymorphism in a region encoding the APCDD1L gene, the R3HCC1 gene, the OR5112 gene, the MKKS gene, the EDEM3 gene, or the ACOX1 gene which are present on genomic DNA in a biological sample collected from a test subject; or a single nucleotide polymorphism which is in linkage disequilibrium with or genetically linked to the single nucleotide polymorphism, and determining whether the single nucleotide polymorphism is homozygous for a variant type, heterozygous, or homozygous for a wild-type. The side effect can be leucopenia or neutropenia.

United States Patent Application Publication No. 2018/0311347 by Lenz discloses methods for treating colorectal cancer patients with irinotecan and bevacizumab when the patients have specific rs1792689, rs2268753, rs17776182, rs7570532 and/or rs4946935 polymorphisms. The polymorphisms are of the group of (G/G) for rs1792689, (C/T) or (C/C) for rs2268753, (G/G) for rs17776182, (A/A) for rs7570532, and (A/G) or (G/G) for rs4946935. The therapy can further comprise administration of folinic acid and/or a pyrimidine analog. The therapy can also further comprise administration of leucovorin and/or 5-fluorouracil. When the patients have a polymorphism that is has (G/G) for rs1792689, (C/T) or (C/C) for rs2268753, (G/G) for rs17776182, (A/A) for rs7570532, and (A/G) or (G/G) for rs4946935, then irinotecan and bevacizumab should not be administered.

United States Patent Application Publication No. 2019/0167661 by Adiwijaya et al. discloses therapies for the treatment of small-cell lung cancer including administration of liposomal irinotecan administered every two weeks. The dose of liposomal irinotecan is 70 mg/m² of free base liposomal irinotecan. In one alternative, the therapy comprises the steps of: (a) preparing a pharmaceutically acceptable injectable composition by combining a dispersion of liposomal irinotecan containing 4.3 mg irinotecan free base/mL of the dispersion with a 5% Dextrose Injection (D5W) or 0.9% Sodium Chloride Injection to obtain the injectable composition having a final volume of 500 mL and 70 mg/m² (free base) of the liposomal irinotecan (±5%); and (b) administering the injectable composition from step (a) containing the irinotecan liposome to the patient in a 90-minute infusion. In some alternatives, dexamethasone and a 5-HT3 blocker can be administered to the subject prior to each administration of the antineoplastic therapy and an anti-emetic can also be administered. Typically, the liposomal irinotecan has a diameter of about 100 nm. The molecular weight of irinotecan free base is 586.68 g/mol while the molecular weight of irinotecan hydrochloride trihydrate is 677.19 g/mol, so that the conversion factor between irinotecan hydrochloride trihydrate is 0.87. Exclusion criteria are specified; these exclusion criteria include: (i) prior treatment regimens with irinotecan, topotecan, or any other topoisomerase I inhibitor; (ii) patients with large cell neuroendocrine carcinoma; (iii) patients who have had more than one regimen of prior cytotoxic chemotherapy; (iv) patients who have had more than one line of immunotherapy, such as with nivolumab, pembrolizumab, ipilimumab, atezolizumab, tremelimumab and/or durvalumab; (v) patients with a history of immunotherapy-induced colitis; (vi) patients with CNS metastasis including new or progressive brain metastasis following prophylactic and/or therapeutic cranial radiation or symptomatic CNS metastasis; (vi) patents with carcinomatous meningitis; (vii) patients who are unable to discontinue the use of strong CYP3A4 or UGT1A1 inhibitors at least one week or strong CYP3A4 inducers at least two weeks prior to initiation of therapy; or (ix) presence of another active malignancy.

According to United States Patent Application Publication No. 2019/0167661 by Adiwijaya et al., certain subgroups of patients diagnosed with SCLC may optionally be treated with a reduced dose of the liposomal irinotecan, including patients who have higher levels of bilirubin or patients with the UGT1A1*28 7/7 homozygous allele. The reduced dose refers to a dose of less than 90 mg/m² of irinotecan (free base) encapsulated in liposomes administered once every two weeks to the patient receiving the reduced dose. In some examples, the reduced dose can be a dose of 50-90 mg/m², including a reduced dose of 50 mg/m², a reduced dose of 60 mg/m², a reduced dose of 70 mg/m² or a reduced dose of 80 mg/m² irinotecan (free base) administered once every two weeks to patients diagnosed with SCLC and receiving the reduced dose. For those patients who start with 70 mg/m², the first dose reduction should be to 50 mg/m² and then to mg/m². The exact determination of the appropriate dose will be dependent on the observed pharmacokinetics, efficacy, and safety in that subpopulation.

In another alternative disclosed by United States Patent Application Publication No. 2019/0167661 by Adiwijaya et al., a combination of liposomal irinotecan and an immunotherapy can be used for treatment. The immunotherapy can be an antibody binding to alpha-PDL1, alpha-44BB, alpha-CTLA4, or alpha-OX40. Examples of immunotherapy can include atezolizumab, avelimumab, nivolumab, pembrolizumab, ipilimumab, tremelimumab and/or durvalumab.

Instill another alternative disclosed by United States Patent Application Publication No. 2019/0167661 by Adiwijaya et al., the liposomal irinotecan can be administered in combination with: (i) a Chk1-directed therapeutic agent such as prexasertib; (ii) a topoisomerase 2-directed therapeutic agent such as aldozurubicin; (iii) a DNA inhibitor such as lurbinectedin; or a Notch ADC compound such as rovalpituzumab tesirine (Rova-T).

United States Patent Application Publication No. 2019/0167790 by Naumovski discloses a method for treating cancer comprising administering to a subject an effective amount of dilpacimab (ABT-165) in combination with folinic acid, 5-fluorouracil, and irinotecan. Dilpacimab is a dual-variable domain immunoglobulin molecule with dual specificity for both delta-like ligand 4 (DLL4) and vascular endothelial growth factor (VEGF). The cancer to be treated can be gastroesophageal cancer, pancreatic cancer, breast cancer, glioblastoma multiforme, ovarian cancer, or non-small-cell lung cancer. Dilpacimab is a humanized recombinant DVD-Ig molecule with a dual specificity for both human DLL4 and human VEGF. Dilpacimab contains a human IgG1/x isotype with two point mutations that diminish binding to Fc y receptors and complement component C1q, but demonstrates pH-dependent binding to FcRn within the expected range of human IgG1. Dilpacimab exhibits a low ability to stimulate cytokine release by human peripheral blood cells (PBC) from normal donors and is within the expected range of other negative control IgG1 antibodies.

United States Patent Application Publication No. 2020/0115740 by Tsunedomi et al. discloses a method of prediction of the therapeutic effect of irinotecan using a specific genetic polymorphism. The genetic polymorphism is rs1980576 in the gene APCDD1L or a genetic polymorphism in linkage disequilibrium with that polymorphism. This polymorphism is adenine in the wild-type and guanine in the mutant. When this polymorphism is homozygous for wild-type, irinotecan has the strongest therapeutic effect. When the polymorphism is heterozygous with one allele being wild-type and the other allele being mutant, irinotecan has an intermediate therapeutic effect. When the polymorphism is homozygous for the mutant, irinotecan has a lower therapeutic effect.

United States Patent Application Publication No. 2020/0188363 by Kwan et al. discloses pharmaceutical compositions including orally administered irinotecan and a P-gp inhibitor. The P-Gp inhibitor is Compound A:

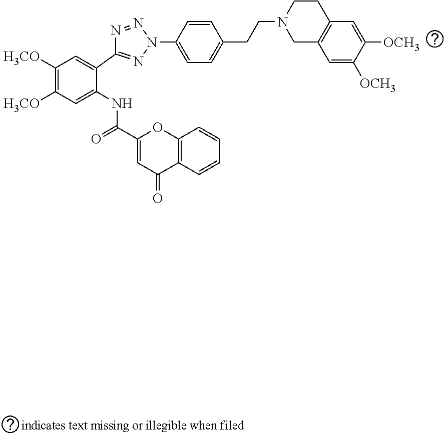 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

This reduces hematologic toxicity, neurotoxicity, skin toxicity, vomiting, diarrhea, fatigue, sensory neuropathy, infection, or hypersensitivity-type reactions associated with infusion.

United States Patent Application Publication No. 2021/0088522 by Sugimoto et al. discloses a marker for determining sensitivity to an anti-cancer agent. The anti-cancer agent includes irinotecan or its metabolite SN-38 or a salt thereof, 5-fluorouracil or a salt thereof, and levofolinate or a salt thereof. The anti-cancer agent can further include an anti-angiogenic drug such as bevacizumab. The marker is one or more of the following molecules: 5-aminoimidazole-4-carboxamide ribotide, alanine, aspartic acid, cysteine, cysteine-glutathione disulfide, glycerol-3-phosphate, histidine, isoleucine, leucine, lysine, methionine sulfoxide, N⁶,N⁶,N⁶-trimethyllysine, N⁶-acetyllysine, octanoic acid, serine, taurocholic acid, threonine, tryptophan, tyrosine, and valine.

Another derivative of irinotecan is ZBH-1208 (Y Hui et al., “Effects of an Irinotecan Derivative, ZBH-1208, on the Immune System in a Mouse Model of Brain Tumor and Its Antitumor Mechanism,” Mol. Med. Rep. 16: 6340-6345 (2017). The structures of irinotecan and ZBH-1208 are shown below:

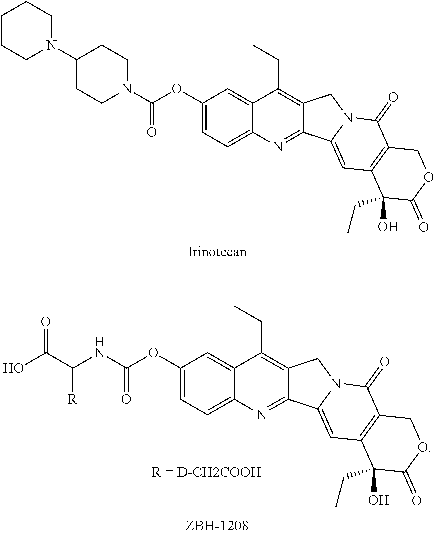

Topotecan has the structure of Formula (XVI):

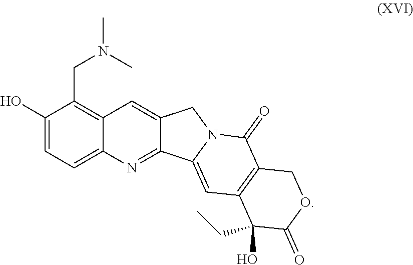 [SMILES: CC[C@@]1(O)C(=O)OCc2c1cc1n(c2=O)Cc2cc3c(CN(C)C)c(O)ccc3nc2-1]

U.S. Pat. No. 5,004,758 to Boehm et al. discloses water-soluble camptothecin analogs, including compounds of Formula (XVII):

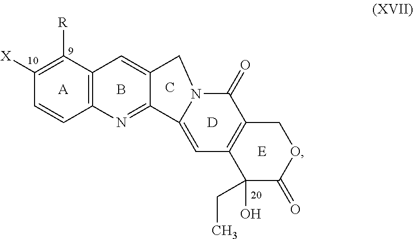

wherein:
(1) X is hydroxy, hydrogen, —CH₂NH₂, or formyl;
(2) R is hydrogen when X is —CH₂NH₂ or formyl, or R is —CHO or —CH₂R¹ when X is hydrogen or hydroxy;
(3) R¹ is —O—R², —S—R², —CH₂NH₂, —N—R²(R³), or —N⁺—R²(R³)(R⁴), provided that when R¹ is —N⁺—R²(R³)(R⁴), the compound is associated with a pharmaceutically acceptable anion;
(4) R², R³, and R⁴ are the same or different and are each independently selected from hydrogen, C₁-C₆ alkyl, C₂-C₆ hydroxyalkyl, C₁-C₆ dialkylamino, C₁-C₆ dialkylamino-C₂-C₆ alkyl, C₁-C₆ dialkylamino-C₂-C₆ alkyl, C₁-C₆ alkylamino-C₂-C₆ alkyl, C₂-C₆ aminoalkyl, or a 3- to 7-membered unsubstituted or substituted carbocyclic ring; and
(5) when R¹ is —N—R²(R³), the R² and R³ groups can be combined together with the nitrogen atom to which they are bonded to form a heterocyclic ring provided that the heterocyclic ring formed is selected from morpholino, N-methylpiperazinyl, or 4′-piperidinopiperidinyl which may contain additional heteroatoms.

U.S. Pat. No. 5,734,056 to Burk et al. discloses methods for preparing water-soluble camptothecin analogs, particularly 9-substituted camptothecins. These compounds include: (20S) 9-N,N-dimethylaminomethyl-10-hydroxycamptothecin; (20S) 9-morpholinomethyl-10-hydroxycamptothecin; (20S) 9-N-methylpiperazinylmethyl-10-hydroxycamptothecin; (20S) 9-(4′-piperidinopiperidinyl)methyl-10-hydroxycamptothecin; (20S) 9-cyclopropylaminomethyl-10-hydroxycamptothecin; (20S) 9-(methylanilinomethyl)-10-hydroxycamptothecin; and (20S) 9-cyclohexylaminomethyl-10-hydroxycamptothecin.

U.S. Pat. No. 6,660,861 to Puri et al. discloses the use of dihalomethanes, particularly dichloromethane, for the preparation of topotecan from 10-hydroxycamptothecin.

U.S. Pat. No. 7,547,785 to Palle et al. discloses a process for producing topotecan acetate comprising the steps of: hydrogenating camptothecin in the presence of a hydrogenation catalyst and thioanisole to form 10-hydroxycamptothecin; and reacting 10-hydroxy camptothecin with dimethylamine and about 1 to about 3 equivalents of formaldehyde, per equivalent of 10-hydroxycamptothecin, in the presence of acetic acid to form topotecan acetate.

U.S. Pat. No. 7,754,733 to Dell'orco et al. discloses a novel crystalline form of topotecan hydrochloride pentahydrate.

U.S. Pat. No. 7,977,483 to Hu et al. discloses a process for preparing topotecan or a pharmaceutically acceptable salt thereof, comprising reacting an iminium salt with 10-hydroxycamptothecin.

U.S. Pat. No. 8,013,158 to Hu et al. discloses several polymorphic crystalline forms of topotecan hydrochloride, including: (i) a crystalline Form D of topotecan hydrochloride having powder X-ray 2θ diffraction peaks at 5.9, 13.9, 22.6, 23.2, and 26.5° (±0.2°); and (ii) a crystalline Form E of topotecan hydrochloride having powder X-ray 2θ diffraction peaks at 14.0, 18.8, 22.5, 25.4, and 25.7° (±0.2°) as well as methods for their preparation.

U.S. Pat. No. 8,026,249 to Czarnik disclosed deuterium-enriched topotecan.

U.S. Pat. No. 8,709,420 to Kumar et al. discloses a pharmaceutical composition of pazopanib and topotecan to treat neuroblastoma, osteosarcoma, or rhabdomyosarcoma.

U.S. Pat. No. 8,828,416 to Falotico et al. discloses the local vascular delivery of topotecan in combination with rapamycin to prevent restenosis following vascular injury. The agents can be delivered by means of a coated stent. Other agents, such as trichostatin, sirolimus, mycophenolic acid, or cladribine, can be used.

United States Patent Application Publication No. 2006/0222694 by Oh et al. discloses a stabilized topotecan liposomal composition that can be reconstituted from a lyophilized form to an injectable liposome suspension having selected liposome sizes in the size range between 0.05 and 0.25 μm, and between about 85%-100% liposome-entrapped topotecan. The liposomes can further comprise a cryoprotectant. Suitable cryoprotectants include sucrose, trehalose, lactose, maltose, cyclodextrin, polyethylene glycol, dextran, polyvinylpyrrolidone, and hydroxyethyl starch. The liposomes can comprise lipids such as cholesterol, phosphatidyl cholines, sphingomyelins, phosphatidylglycerols, phosphatidic acids, phosphatidylethanolamines, phosphatidylinositols, phosphatidylserines, cholesterol sulfate, or cholesterol hemisuccinate. The lipid used may be conjugated to a hydrophilic polymer such as polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and polyglycerol.

United States Patent Application Publication No. 2007/0149783 by Palle et al. discloses a process for producing topotecan acetate comprising reacting 10-hydroxycamptothecin with dimethylamine and about 1-3 equivalents of formaldehyde per equivalent of 10-hydroxycamptothecin in the presence of acetic acid to form topotecan acetate. The topotecan acetate can be isolated by adding an antisolvent such as a ketone, a hydrocarbon, a chlorinated solvent, or an ester. The topotecan acetate can be converted to topotecan hydrochloride by reaction with hydrochloric acid. Crystalline forms of topotecan hydrochloride are also disclosed.

United States Patent Application Publication No. 2009/0192184 by Pozzi et al. discloses two crystalline forms of topotecan hydrochloride, designated the α and β forms, and characterized by X-ray powder diffraction spectra. The a form can be produced by: (i) reaction of 10-hydroxycamptothecin with an excess of aqueous formaldehyde and aqueous dimethylamine in acetic acid and a straight or branched C₂-C₄ alcohol; (ii) addition of hydrochloric acid; (iii) concentration of the reaction mixture; (iv) crystallization of topotecan hydrochloride form by addition of isopropanol or aqueous isopropanol; and (v) recovery of the resulting topotecan hydrochloride form a. Additionally, the p form, with a distinct X-ray powder diffraction pattern, can be produced from the a form by the following steps: (i) suspension of form a in aqueous isopropanol at a temperature ranging from 48 to 52° C. for at least 60 minutes to obtain a crystalline suspension; (ii) cooling of the crystalline suspension at a temperature ranging from 15 to 25° C.; and (iii) recovery of topotecan hydrochloride form p.

United States Patent Application Publication No. 2009/0221622 by Teja et al. discloses a stabilized topotecan-containing composition comprising: (a) topotecan or a pharmaceutically acceptable salt thereof; and (b) a pharmacologically suitable fluid comprising an aqueous diluent, wherein: (i) the pH of the composition is less than or equal to about 1.5; and (ii) the composition is stable during long term storage; wherein the 10-hydroxycamptothecin (10-HCPT) resulting from the degradation of the topotecan during the long term storage does not precipitate in the pharmaceutically suitable fluid until the 10-hydroxycamptothecin (10-HCPT) reaches a concentration of about 6 μg/mL. The aqueous diluent can include an acid selected from the group consisting of hydrochloric acid, methanesulfonic acid, and trifluoroacetic acid. The composition can further include benzyl alcohol. In another alternative, the composition can include a hydroxyacid selected from the group consisting of hydroxy carboxylic acids and hydroxy tricarboxylic acids; a preferred hydroxyacid is lactic acid.

United States Patent Application No. 2014/0371258 by Gu et al. discloses a water-soluble conjugate of topotecan having two or more molecules of topotecan covalently attached to a water-soluble polymer. The two or more topotecan molecules can be releasably attached to the polymer.

In one alternative, the water-soluble conjugate has the formula:

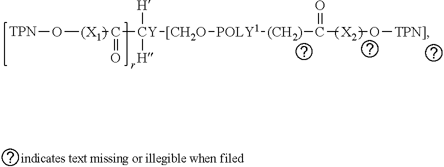

wherein:
(1) y is 0 or 1, such that when y is 0, —C_{y}H′H″ is absent, and when y is 1, C_{y} is present;
(2) m is a positive integer from 1 to about 12;
(3) X₁ and X₂, when present, are each an amino acid linker, such that the amino acid carbonyl carbon of the linker is adjacent to the oxygen in the TPN-O moiety;
(4) each POLY¹ is a water-soluble, non-peptide polymer:
(5) q is 1, 2, 3, or 4;
(6) r is 0 or 1;
(7) “TPN-O” is the following moiety:

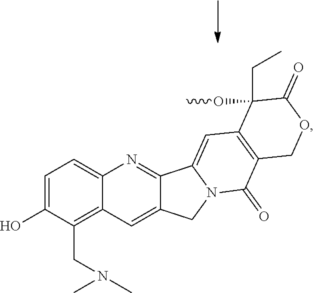

(8) when r is 1, q does not equal 4;
(9) when r is 0, q is 2, 3, or 4; and
(10) when r+q does not equal 4, then H′ and optionally H″ are present to bring the valence on C_{y} to 4.

In one specific alternative, the structure comprises:

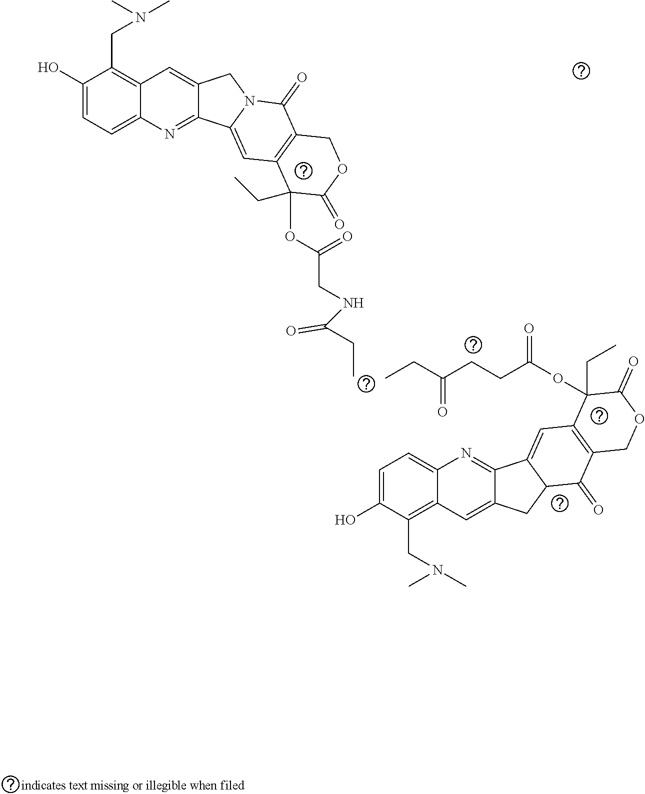

In this alternative, n is from 10 to 1500, more preferably from 200 to 800.

In some alternatives, POLY¹ is a water-soluble and non-peptidic polymer selected from the group consisting of poly(alkylene glycol), poly(olefinic alcohol), poly(vinylpyrrolidone), poly(hydroxyalkylmethacrylamide), poly(hydroxyalkylmethacrylate), poly(saccharide), poly(α-hydroxy acid), poly(acrylic acid), poly(vinyl alcohol), polyphosphazene, polyoxazoline, poly(N-acryloylmorpholine), or copolymers or terpolymers thereof; the polymer can be, for example, polyethylene glycol.

The linker can include amino acid linkers. Typically, the amino acid linkers are formed from alanine, valine, leucine, isoleucine, glycine, threonine, serine, cysteine, methionine, tyrosine, phenylalanine, tryptophan, aspartic acid, glutamic acid, lysine, arginine, histidine, proline, or non-naturally occurring amino acids. Preferably, the amino acid linkers are alanine, glycine, isoleucine, leucine, phenylalanine, or valine. More preferably, the amino acid linkers are glycine.

Typically, the polymer has from 2 to 4 arms, wherein each arm has one topotecan moiety.

Suitable salts and solvates of irinotecan include, but are not limited to, irinotecan hydrochloride; irinotecan sulfate; irinotecan nitrate; irinotecan phosphate; irinotecan methanesulfonate; irinotecan citrate; irinotecan maleate; irinotecan succinate; irinotecan disulfate; irinotecan malate; irinotecan fumarate; irinotecan besylate; irinotecan camsylate; irinotecan edisylate; and irinotecan hydrochloride trihydrate.

The following additional therapeutic agents or combinations of additional therapeutic agents have been described as suitable for use with irinotecan.

The therapeutic agent thalidomide has been described as suitable for use with irinotecan for the treatment of colorectal cancer.

The combination of 5-fluorouracil and leucovorin has been described as suitable for use with irinotecan for the treatment of pancreatic cancer.

The combination of trifluridine and tipiracil hydrochloride has been described as suitable for use with irinotecan for the treatment of colorectal cancer, lung cancer, breast cancer, pancreatic cancer, or gastric cancer.

The combination of aflibercept, folinic acid, and 5-fluorouracil has been described as suitable for use with irinotecan for the treatment of colorectal cancer.

The combination of oxaliplatin, 5-fluorouracil, and leucovorin has been described as suitable for use with irinotecan for the treatment of gastric cancer.

The therapeutic agent 5-(5-(2-(3-aminopropoxy)-6-methoxyphenyl)-1H-pyrazol-3-ylamino)pyrazine-2-carbonitrile has been described as suitable for use with irinotecan for the treatment of rhabdomyosarcoma.

The combination of tegafur, uracil, and folinic acid has been described as suitable for use with irinotecan. The tegafur and uracil produce the antimetabolite 5-fluorouracil in vivo.

EGFR inhibitors such as, but not limited to, erlotinib have been described as suitable for use with irinotecan.

VEGF inhibitors such as Flt1D2.Flk1D3.FcΔC1 have been described as suitable for use with irinotecan.

A humanized anti-EGFR IgG1 antibody has been described as suitable for use with irinotecan.

The therapeutic agent 4-iodo-3-nitrobenzamide and metabolites thereof, including 4-iodo-3-aminobenzoic acid and 4-iodo-3-aminobenzamide, have been described as suitable for use with irinotecan for the treatment of metastatic breast cancer.

The therapeutic agent bevacizumab has been described as suitable for use with irinotecan for the treatment of colorectal cancer.

Immunotherapies including: (i) an antibody binding to alpha-PDL1, alpha-44BB, alpha-CTLA4, or alpha-OX40; or atezolizumab, avelimumab, nivolumab, pembrolizumab, ipilimumab, tremelimumab, or durvalumab; (ii) a Chk1-directed therapeutic agent such as prexasertib; (iii) a topoisomerase 2-directed therapeutic agent such as aldozurubicin; (iv) a DNA inhibitor such as lurbinectedin; or (v) a Notch ADC compound such as rovalpituzumab tesirine have been described as suitable for use with irinotecan.

The combination of dilpacimab, folinic acid, and 5-fluorouracil has been described as suitable for use with irinotecan for the treatment of gastroesophageal cancer, pancreatic cancer, breast cancer, glioblastoma multiforme, ovarian cancer, or non-small-cell lung cancer.

MRP inhibitors including valspodar (SDZ-PSC 833), tert-butyl 2-[(3S,6S,9S,15S,21S,24S,27S,30S)-15,18-bis[(2S)-butan-2-yl]-6-[(4-methoxyphenyl)methyl]-3,10,16,19,22,28-hexamethyl-2,5,8,11,14,17,20,23,26,29-decaoxo-9,24,27-tri(propan-2-yl)-4-oxa-1,7,10,13,16,19,22,25,28-nonazabicyclo[28.4.0]tetratriacontan-21-yl]acetate (SDZ 280-446), sodium 3-[[3-[(E)-2-(7-chloroquinolin-2-yl)ethenyl]phenyl]-[3-(dimethylamino)-3-oxopropyl]sulfanylmethyl]sulfanylpropanoate (MK571), dofequidar (MS209), 2-(4-benzhydrylpiperazin-1-yl)ethyl 5-[(4R,6R)-4,6-dimethyl-2-oxo-1,3,2λ-5-dioxaphosphinan-2-yl]-2,6-dimethyl-4-(3-nitrophenyl)pyridine-3-carboxylate (PAK-104p), verapamil, benzbromarone, dipyridamole, furosemide, gamma-GS(naphthyl)cysteinyl-glycine diethyl ester, genistein, quinidine, rifampicin, mifepristone (RU-486), or sulfinpyrazone have been described as suitable for use with irinotecan.

The following agents can be used for pre-treatment or post-treatment to reduce side effects associated with administration of irinotecan.

The agents tamoxifen, loperamide, baicalin, or octreotide, as well as antibiotics, can be used for the prevention of diarrhea. Alternatively, an antiestrogen, which can be droloxifene, miproxifene phosphate (TAT-59), or raloxifene, can be used to the prevention of diarrhea.

Yet another agent that can reduce hematologic toxicity, neurotoxicity, skin toxicity, vomiting, diarrhea, fatigue, sensory neuropathy, infection, or hypersensitivity-type reactions associated with infusion of irinotecan is the P-Gp inhibitor Compound A whose formula is shown below:

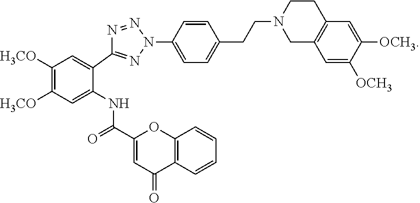 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

The following phenotypic or genomic markers are associated with either the efficacy of irinotecan administration or the occurrence or severity of side effects associated with irinotecan administration.

The upregulation of genes for ERBB2, GRB7, JNK1 kinase, BCL2, MK167, phospho-Akt, CD-68 and BAG1 is associated with the responsiveness to treatment of colorectal cancer with irinotecan. The downregulation of genes for Erk1 kinase, phospho-GSK-3β, MMP11, CTSL2, CCNB1, BIRC5, STK6, MRP14 and GSTM1 is also associated with the responsiveness to treatment of colorectal cancer with irinotecan.

A genotypic marker at position −3156 of the UGT1A1 gene or at any position in linkage disequilibrium with position −3156 of the UGT1A1 is correlated with irinotecan toxicity. An A at that position positively correlates with irinotecan toxicity, while a G at that position correlates with tolerance to irinotecan and reduced toxicity. If the subject is homozygous at that position with A at both alleles, the risk of toxicity increases. This toxicity is associated with a reduction of glucuronidation of the active irinotecan metabolite SN-38.

Other genomic markers are associated with polymorphisms in the TATA box within the promoter region of the UDP glucuronosyl transferase gene UGT1A1. Polymorphisms that predispose to serious side effects associated with the administration of irinotecan have 7 TA repeats in the TATA box within the promoter region rather than 6 TA repeats in the wild-type promoter. This lowers the gene expression of UGT1A1 and results in lower UDP glucuronosyl transferase activity. A reduction in UDP glucuronosyl transferase activity can increase the risk of side effects associated with administration of irinotecan such as diarrhea. Patients with 7 TA repeats in the TATA box who have been diagnosed with small-cell lung cancer should receive a reduced dose of irinotecan.

Expression of the following genes: AMD1, CTSC, EIF1AX, C12orf30, DDX54, PTPN2, and TBX3 can affect the therapeutic efficacy of irinotecan.

The following additional genotypic or phenotypic factors have also been shown to affect the therapeutic efficacy of irinotecan: (i) mutation of topoisomerase I; (ii) the expression level of topoisomerase I; (iii) the activity of carboxylesterase; (iv) the activity of ABC transporter genes including the genes encoding multidrug resistance proteins (MRP) MRP-1 and MRP-2 and breast cancer resistant protein BCRP encoded by the gene ABCG2; and (v) the plasma level of tissue inhibitor of metalloproteinase-1 (TIMP-1). The existence of variant alleles of the gene MRP1 which encodes the multidrug resistance protein MRP-1 also affects the therapeutic efficacy of irinotecan.

Single nucleotide polymorphisms in a region encoding the APCDD1L gene, the R3HCC1 gene, the OR5112 gene, the MKKS gene, the EDEM3 gene, or the ACOX1 gene also affect the efficacy of irinotecan.

Certain additional polymorphisms also affect the suitability of the administration of irinotecan with bevacizumab for the treatment of colorectal cancer. Specifically, these polymorphisms are the following: rs1792689, rs2268753, rs17776182, rs7570532 and/or rs4946935 polymorphisms. The polymorphisms are of the group of (G/G) for rs1792689, (C/T) or (C/C) for rs2268753, (G/G) for rs17776182, (A/A) for rs7570532, and (A/G) or (G/G) for rs4946935. The therapy can further comprise administration of folinic acid and/or a pyrimidine analog. The therapy can also further comprise administration of leucovorin and/or 5-fluorouracil. When the patients have a polymorphism that is has (G/G) for rs1792689, (C/T) or (C/C) for rs2268753, (G/G) for rs17776182, (A/A) for rs7570532, and (A/G) or (G/G) for rs4946935, then irinotecan and bevacizumab should not be administered. Another polymorphism associated with the therapeutic effect of irinotecan is rs1980576 in the gene APCDD1L or a genetic polymorphism in linkage disequilibrium with that polymorphism. This polymorphism is adenine in the wild-type and guanine in the mutant. When this polymorphism is homozygous for wild-type, irinotecan has the strongest therapeutic effect. When the polymorphism is heterozygous with one allele being wild-type and the other allele being mutant, irinotecan has an intermediate therapeutic effect. When the polymorphism is homozygous for the mutant, irinotecan has a lower therapeutic effect.

The following additional therapeutic agents or combinations of additional therapeutic agents have been described as suitable for use with topotecan.

The agent pazopanib has been described as suitable for use with topotecan to treat neuroblastoma, osteosarcoma, or rhabdomyosarcoma.

The agents rapamycin, trichostatin, sirolimus, mycophenolic acid, and cladribine has been described as suitable for use with topotecan to prevent restenosis following vascular injury.

In general, this invention is directed to novel compositions and methods to improve the utility of therapeutic agents with suboptimal performance in patients with cancer, infections, immunological diseases and other diseases and conditions as stated below. In particular, the present invention describes: novel improvements; pharmaceutical ingredients and formulations; dosage forms; excipients; solvents; diluents; drug delivery systems; preservatives; methods for administration including improved dose determination, dosage schedules, routes of administration, or durations of administration; toxicity monitoring or amelioration; phenotypic or genotypic determination to identify patients who might achieve a better outcome with administration of the therapeutic agents, either by increased therapeutic efficacy or reduced side effects or toxicity; or pharmacokinetic or metabolic monitoring approaches. In particular, the present invention also describes the use of drug delivery systems, prodrugs, polymer conjugates, drug combinations, or multiple drug systems. The present invention further describes the use of these therapies in conjunction with radiation, other conventional therapeutic agents, or biotherapeutic agents such as antibodies, vaccines, cytokines, lymphokines, gene therapies, antisense RNA therapies, small interfering RNA (siRNA) therapies, or other biotherapeutic agents. The present invention, therefore, provides novel approaches to the use of these agents that can either improve therapeutic efficacy or reduce toxicity or side effects that are associated with administration of these agents. These compositions and methods can potentiate the activity of the compounds or inhibit the repair of suboptimal cellular effects or sub-lethal damage or to “push” the cell into more destructive cellular phases such as apoptosis or other lethalities.

Examples of suboptimal therapeutics can include many classes of therapeutic agents, including, but not limited to, antimetabolites, DNA/nucleic acid binding/reactive agents, topoisomerase inhibitors, anti-tubulin agents, signal transduction inhibitors, protein synthesis inhibitors, inhibitors of DNA transcribing enzymes, DNA/RNA intercalating agents, DNA minor groove binders, drugs that block steroid hormone action, photochemically active agents, immune modifying agents, hypoxia selective cytotoxins, chemical radiation sensitizers and protectors, antisense nucleic acids, oligonucleotides and polynucleotides as therapeutic agents, immune modifying agents, antitumor antibiotics, biotherapeutics, and biologic agents such as cancer vaccines, antibody therapies, cytokines, lymphokines, gene therapies, nucleic acid therapies, and cellular therapies.

These agents include substituted camptothecins, including irinotecan, topotecan, and derivatives and analogs of irinotecan or topotecan, as well as other substituted camptothecins.

In the inventive compositions and methods, the term “suboptimal therapy” includes agents or combinations of agents where Phase I toxicity precluded further human clinical application. It also includes agents that had undergone Phase II trials with limited (<25%) response rates or with no significant treatment responses. It also includes agents that had been the subject of Phase III clinical trials in which the outcome was either medically or statistically not significant to warrant regulatory submission or approval by government agencies for commercialization for commercialized agents whose clinical performance (i.e., response rates) as a monotherapy are less than 25%, or whose side effects are severe enough to limit wide utility.

Examples of compounds with suboptimal therapeutic activity include many classes of compounds as described above and many compounds included within these classes.

### I. Substituted Camptothecins, Including Irinotecan, Topotecan, and Derivatives and Analogs of Irinotecan or Topotecan

Substituted camptothecins within the scope of the present invention and usable in methods and compositions according to the present invention include irinotecan, topotecan, and derivatives and analogs of irinotecan or topotecan as described above.

Camptothecins within the scope of the present invention are cytotoxic alkaloids. The molecular action of irinotecan occurs by trapping a subset of topoisomerase-1-DNA cleavage complexes, those with a guanine+1 in the DNA sequence. One irinotecan molecule stacks against the base pairs flanking the topoisomerase-induced cleavage site and poisons (inactivates) the topoisomerase 1 enzyme.

Irinotecan has the structure of Formula (I):

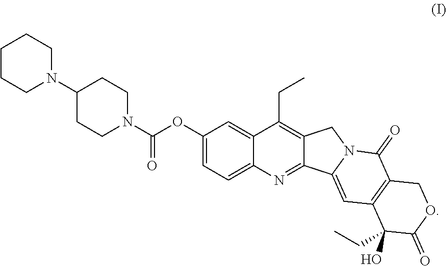 [SMILES: CCc1c2c(nc3ccc(OC(=O)N4CCC(N5CCCCC5)CC4)cc13)-c1cc3c(c(=O)n1C2)COC(=O)[C@]3(O)CC]

The IUPAC systemic name for irinotecan is (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate.

As detailed below, irinotecan acts in vivo as a prodrug, and is hydrolyzed to its active metabolite SN-38, shown below as Formula (II):

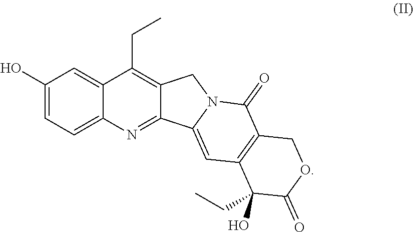 [SMILES: CCc1c2c(nc3ccc(O)cc13)-c1cc3c(c(=O)n1C2)COC(=O)[C@]3(O)CC]

Irinotecan is hydrolyzed in the liver to SN-38 by two carboxylesterase converting enzymes, CES1 and CES2, and is also hydrolyzed in the plasma by butyrylcholinesterase.

Irinotecan can exist in a variety of salts and solvates. These salts and solvates include, but are not limited to: irinotecan hydrochloride; irinotecan sulfate; irinotecan nitrate; irinotecan phosphate; irinotecan methanesulfonate; irinotecan citrate; irinotecan maleate; irinotecan succinate; irinotecan disulfate; irinotecan malate; irinotecan fumarate; irinotecan besylate; irinotecan camsylate; irinotecan edisylate; and irinotecan hydrochloride trihydrate. Irinotecan can also exist as a free base.

A variety of polymorphic crystalline forms of irinotecan have been identified. These polymorphic forms are disclosed in: U.S. Pat. No. 7,435,818 to Chen et al.; U.S. Pat. No. 7,488,825 to Shimizu et al.; U.S. Pat. No. 8,247,426 to Pozzi et al.; U.S. Pat. No. 10,919,905 to Liao et al.; and United States Patent Application Publication No. 2006/0046993 by Forino et al.

Methods for preparing irinotecan or a salt or solvate thereof are disclosed in: U.S. Pat. No. 7,683,170 to Wissmann et al.; U.S. Pat. No. 9,765,083 by Zabudkin et al.; United States Patent Application Publication No. 2007/0208050 by Palle et al.; and United States Patent Application Publication No. 2008/0182990 by Vishnukant et al.

Topotecan has the structure of Formula (XVI):

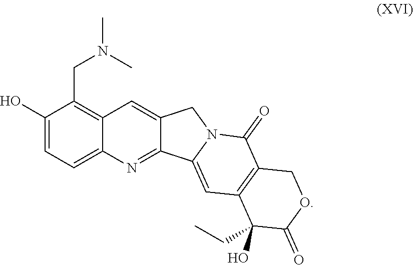 [SMILES: CC[C@@]1(O)C(=O)OCc2c1cc1n(c2=O)Cc2cc3c(CN(C)C)c(O)ccc3nc2-1]

The IUPAC name for topotecan is (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride.

U.S. Pat. No. 6,660,861 to Puri et al. discloses methods for preparing topotecan. U.S. Pat. No. 7,547,785 to Palle et al. discloses methods for preparing topotecan acetate. U.S. Pat. No. 7,977,483 to Hu et al. discloses methods for preparing topotecan or salts thereof.

Crystalline forms, including polymorphs, of topotecan and salts thereof are disclosed in U.S. Pat. No. 7,754,733 to Dell'orco et al. (topotecan acetate); U.S. Pat. No. 8,013,158 to Hu et al. (topotecan hydrochloride); and United States Patent Application Publication No. 2009/0192814 by Pozzi et al. (topotecan hydrochloride).

Other substituted camptothecins within the scope of the present invention include, but are not limited to, belotecan, diflomotecan, exatecan, lurtotecan, and rubitecan.

Still other substituted camptothecins within the scope of the present invention include, but are not limited to, hydroxymethylcamptothecin, 5-hydroxycamptothecin, 20-O-acetyl-7-acetoxymethylcamptothecin, 7-acetoxymethylcamptothecin, 7-succinoyloxymethylcamptothecin, 20-O-trifluoroacetyl-7-trifluoroacetoxymethylcamptothecin, 7-benzoyloxymethylcamptothecin, 7-propionyloxymethylcamptothecin, 7-butyryloxymethylcamptothecin, 7-caprylyloxymethylcamptothecin, 7-capryloxymethylcamptothecin, 7-isovaleryloxymethylcamptothecin, 7-phenylacetoxymethylcamptothecin, camptothecin-7-carboxylic acid, ethyl camptothecin-7-carboxylate, 5-methoxycamptothecin, 5-butoxycamptothecin, 5-acetoxycamptothecin, 20-O-acetyl-5-acetoxycamptothecin, 5-benzoyloxycamptothecin, 7-methylcamptothecin, 7-ethylcamptothecin, 7-propylcamptothecin, 7-butylcamptothecin, 7-heptylcamptothecin, 7-nonylcamptothecin, 7-isobutylcamptothecin, 7-benzylcamptothecin, 7-β-phenethylcamptothecin, 7-isopropylcamptothecin; 7-cyclohexylcamptothecin; 9-chlorocarbonyloxy-7-ethyl-camptothecin; 10-chlorocarbonyloxy-camptothecin; 10-chlorocarbonyloxy-7-ethyl-camptothecin; 11-chlorocarbonyloxy-camptothecin; 11-chlorocarbonyloxy-7-ethyl-camptothecin; 7-ethyl-9-[4-(N-isopropylcarbamoylmethyl)-1-piperazino]carbonyloxy-camptothecin; 9-(1-piperazino)carbonyloxy-camptothecin; 9-(4-methyl-1-piperazino)carbonyloxy-camptothecin; 9-[4-(N-isopropylcarbamoylmethyl)-1-piperazino]carbonyloxy-camptothecin; 9-[4-(1-piperidino)-1-piperidino]carbonyloxy-camptothecin; 9-[N-methyl-N-(2-dimethylaminoethyl)]carbonyloxy-camptothecin; 7-ethyl-9-(1-piperazino)carbonyloxy-camptothecin; 7-ethyl-9-(4-methyl-1-piperazino)carbonyloxy-camptothecin; 7-ethyl-9-[4-(N-isopropylcarbamoylmethyl)-1-piperazino]carbonyloxy-camptothecin; 7-ethyl-9[4-(1-piperidino)-1-piperidino]carbonyloxy-camptothecin; 7-ethyl-9-[N-propyl-N-(2-dimethylaminoethyl)]carbonyloxy-camptothecin; 9-(1-piperazino)carbonyloxy-7-propyl-camptothecin; 10-[(N-ethoxycarbonylmethylamino)carbonyloxy]-7-ethyl-camptothecin; 10-(2-diethylamino)-ethyl-aminocarbonyloxy-7-ethyl-camptothecin; 10-diethylaminocarbonyloxy-7-ethyl-camptothecin; 7-ethyl-10-(4-morpholino)carbonyloxy-camptothecin; 7-ethyl-10-(1-piperazino)carbonyloxy-camptothecin; 7-ethyl-10-(4-methyl-1-piperazino)carbonyloxy-camptothecin; 7-ethyl-10-(4-ethyl-1-piperazino)carbonyloxy-camptothecin; 10-(4-benzyl-1-piperazino)carbonyloxy-7-ethyl-camptothecin; 7-ethyl-10-[4-(p-methoxyphenyl)-1-piperazino]carbonyloxy-camptothecin; 7-ethyl-10-[4-(3-hydroxypropyl)-1-piperazino]carbonyloxy-camptothecin; 7-ethyl-10-[4-(N-isopropylcarbamoylmethyl)-1-piperazino]carbonyloxy-camptothecin; 7-ethyl-10-[4-(1-piperidino)piperidino]carbonyloxy-camptothecin; 7-ethyl-10-[N-methyl-N-(2-dimethylaminoethyl)]aminocarbonyloxy-camptothecin; 7-ethyl-10-N-methyl-N-(1-methyl-4-piperidino)aminocarbonyloxy-camptothecin; 10-(4-morpholino)carbonyloxy-camptothecin; 10-(4-methyl-1-piperazino)carbonyloxy-camptothecin; 7-ethyl-10-(4-propyl-1-piperazino)carbonyloxy-camptothecin; 7-ethyl-10-(4-methyl-1-piperazino)carbonyloxy-camptothecin; 11-(4-ethyl-1-piperazino)carbonyloxy-camptothecin; 11-[4-(1-piperidino)-1-piperidino]carbonyloxy-camptothecin; 11-(1-piperazino)carbonyloxy-camptothecin; 11-(4-methyl-1-piperazino)carbonyloxy-camptothecin; 11-[4-(N-isopropylcarbamoylmethyl)-1-piperazino]carbonyloxy-camptothecin; 11-[N-methyl-N-(2-dimethylaminoethyl)]carbonyloxy-camptothecin; 7-ethyl-11-(1-piperazino)carbonyloxy-camptothecin; 7-ethyl-11-(4-methyl-1-piperazino)carbonyloxy-camptothecin; 7-ethyl-11-[4-(N-isopropylcarbamoylmethyl)-1-piperazino]carbonyloxy-camptothecin; 7-ethyl-11-[N-methyl-N-(2-dimethylaminoethyl)]carbonyloxy-camptothecin; and 7-ethyl-11-[4-(1-piperidino)-1-piperidino]carbonyloxy-camptothecin. Still other substituted camptothecins and derivatives and analogs thereof are within the scope of the invention.

U.S. Pat. No. 4,399,276 to Miyazawa et al. discloses 7-substituted camptothecin derivatives of Formula (C-I):

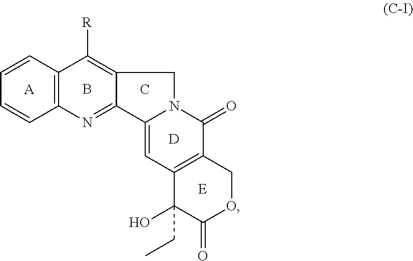

wherein:
(1) R is —CHO, —CH₂OR′, —CH(OR′)₂, or —CH═N—X;
(2) R′ is C₁-C₆ lower alkyl, phenyl(C₁-C₃) alkyl;
(3) X is hydroxyl or —NR¹R², where R¹ and R² are the same or different and where each is hydrogen or C₁-C₆ lower alkyl or, when R¹ is hydrogen, R² may be C₁-C₆ lower alkyl, a substituted or unsubstituted aryl group, a carbamoyl group, an acyl group, an aminoalkyl group, or an amidino group, or where R¹ is a lower alkyl group, R² may be an aminoalkyl group, or R¹ and R² may be combined together with the nitrogen atom to form a heterocyclic group. The compounds described in the reference include camptothecin-7-aldehyde, camptothecin-7-aldehyde oxime, camptothecin-7-aldehyde hydrazone, camptothecin-7-aldehyde hydrazone, camptothecin-7-aldehyde p-toluenesulfonylhydrazone, camptothecin-7-CH═N—N═C(NH₂)₂, camptothecin-7-CH═N—NH—COCH₂—N(CH₃)₂·HCl, camptothecin-7-CH═N—NH—COCH₂—N(CH₃)₃·Cl, camptothecin 7-aldehyde semicarbazone, camptothecin 7-aldehyde phenylsemicarbazone, camptothecin 7-aldehyde thiosemicarbazone, and camptothecin derivatives of Formulas (C-II), (C-III), (C-IV), (C-V), and (C-VI):

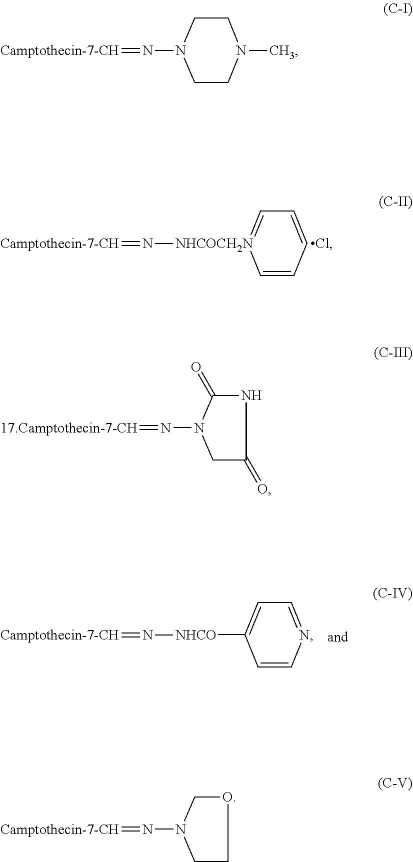

U.S. Pat. No. 4,399,282 to Miyazawa et al. discloses camptothecin derivatives of Formula (C-VII):

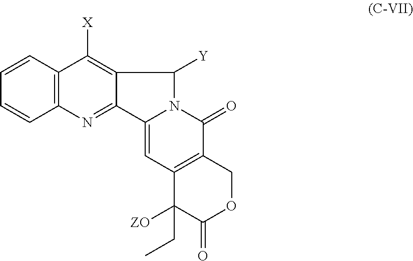

wherein:
(1) X is hydrogen, CH₂OH, carboxyl, alkyl, aralkyl, CH₂OR¹, or CH₂OR²;
(2) R¹ is an alkyl group or an acyl group;
(3) R² is a lower alkyl group;
(4) Y is hydrogen, hydroxyl, or OR³, wherein R³ is a lower alkyl group or an acyl group;
(5) Z is hydrogen or an acyl group;
   with the proviso that when X is CH₂OH, an alkyl group or an aralkyl group, both Y and Z are H; that when X is CH₂OR¹ or CH₂OR², Y is H; that when Y is hydroxyl, both X and Z are H; and that when Y is OR³, X is H.

U.S. Pat. No. 4,604,463 to Miyazawa et al. discloses various camptothecin derivatives and methods for producing the camptothecin derivatives. Camptothecin itself is characterized by a pentacyclic structure consisting of quinoline (rings A and B), pyrroline (ring C), α-pyridone (ring D), and a six-membered lactone (ring E). The camptothecin derivatives are of the general formula (C-VIII):

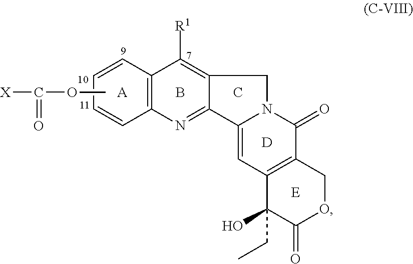

wherein R₁ is hydrogen, halogen, or C₁-C₄ alkyl; X is chlorine or —NR²R³ where R² and R³ are the same or different and each of R² and R³ is hydrogen or a substituted or unsubstituted C₁-C₄ alkyl or a substituted or unsubstituted carbocyclic or heterocyclic group, with the proviso that when both R² and R³ are substituted or unsubstituted alkyl groups, they may be combined together with the nitrogen atom to which R² and R³ are bonded to form a heterocyclic ring which may be interrupted with —O—, —S—, and/or >N—R⁴ in which R⁴ is hydrogen, a substituted or unsubstituted C₁-C₄ alkyl or a substituted phenyl group, and wherein the grouping —O—CO—X is bonded to a carbon atom located in any of the 9-, 10-, or 11-positions in the A ring of the camptothecin moiety.

U.S. Pat. No. 5,004,758 to Boehm et al. discloses water-soluble camptothecin analogs, including compounds of Formula (C-IX):

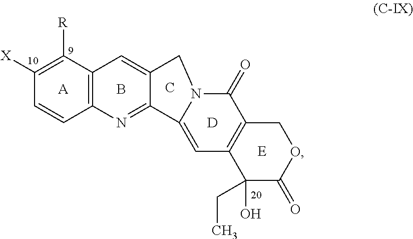

wherein:
(1) X is hydroxy, hydrogen, —CH₂NH₂, or formyl;
(2) R is hydrogen when X is —CH₂NH₂ or formyl, or R is —CHO or —CH₂R¹ when X is hydrogen or hydroxy;
(3) R¹ is —O—R², —S—R², —CH₂NH₂, —N—R²(R³), or —N⁺—R²(R³)(R⁴), provided that when R¹ is —N⁺—R²(R³)(R⁴), the compound is associated with a pharmaceutically acceptable anion;
(4) R², R³, and R⁴ are the same or different and are each independently selected from hydrogen, C₁-C₆ alkyl, C₂-C₆ hydroxyalkyl, C₁-C₆ dialkylamino, C₁-C₆ dialkylamino-C₂-C₆ alkyl, C₁-C₆ dialkylamino-C₂-C₆ alkyl, C₁-C₆ alkylamino-C₂-C₆ alkyl, C₂-C₆ aminoalkyl, or a 3- to 7-membered unsubstituted or substituted carbocyclic ring; and
(5) when R¹ is —N—R²(R³), the R² and R³ groups can be combined together with the nitrogen atom to which they are bonded to form a heterocyclic ring provided that the heterocyclic ring formed is selected from morpholino, N-methylpiperazinyl, or 4′-piperidinopiperidinyl which may contain additional heteroatoms.

### II. Dose Modification

Improvements for suboptimal therapeutics including substituted camptothecins such as, but not limited to, irinotecan and topotecan are made by alterations to the time that the compound is administered, the use of dose-modifying agents that control the rate of metabolism of the compound, use of agents protective of normal tissue, and other dose modifications. General examples include: variations of infusion schedules (e.g., bolus i.v. versus continuous infusion), dose modifications associated with the use of lymphokines (e.g., G-CSF, GM-CSF, EPO) to increase leukocyte count or prevent anemia caused by myelosuppressive agents, dose modifications associated with the use of rescue agents such as leucovorin for 5-FU or thiosulfate for cisplatin treatment. Specific inventive examples for substituted camptothecins such as irinotecan and topotecan include: intravenous infusion for hours to days; biweekly, tri-weekly, or monthly administration; doses greater than 100 mg/m²/day; progressive escalation of dosing from 100 mg/m²/day based on patient tolerance; doses less than 2 mg/m² for greater than 14 days; dose modification associated with use of polyamine to modulate metabolism; dose modification associated with use of eflornithine to modulate metabolism; selected and intermittent boost dose administration; bolus single and multiple doses escalating from 100 mg/m²; oral doses below 30 or above 130 mg/m²; low potency (1-10 mg/mL) oral solutions or suspensions; and medium potency (10-200 mg/mL) oral solutions or suspensions.

### III. Route of Administration

Improvements for suboptimal therapeutics including substituted camptothecins such as, but not limited to, irinotecan and topotecan are made by alterations in the route that the compound is administered. General examples include: changing the route of administration from oral to intravenous administration or vice versa, or the use of specialized routes such as subcutaneous, intramuscular, intraarterial, intraperitoneal, intralesional, intralymphatic, intratumoral, intrathecal, intravesicular, or intracranial. Specific inventive examples for substituted camptothecins such as irinotecan and topotecan include: topical administration; intravesicular administration for bladder cancer; oral administration; slow release oral delivery; intrathecal administration; intraarterial administration; continuous infusion; intermittent infusion; administration by use of large-volume oral solutions; buccal administration; or rectal administration.

### IV. Schedule of Administration

Improvements for suboptimal therapeutics including substituted camptothecins such as, but not limited to, irinotecan and topotecan are made by alterations to the time that the compound is administered. General examples include: changing from a monthly administration to a weekly or daily dosing or variations of the schedule. Specific inventive examples for substituted camptothecins such as irinotecan and topotecan include: daily administration; weekly administration for three weeks; weekly administration for two weeks; biweekly administration; biweekly administration for three weeks with a 1-2 week rest period; intermittent boost dose administration; administration daily for one week then once per week for multiple weeks; or administration daily on days 1-5, 8-12 every three weeks, 2-5 times per day.

### V. Indications for Use

Improvements for suboptimal therapeutics including substituted camptothecins such as, but not limited to, irinotecan and topotecan are made by alterations in the types of disease, clinical stage of disease that the compound is administered. General examples include: the use of solid tumor agents for leukemias and vice versa, the use of antitumor agents for the treatment of benign hyperproliferative disease such as psoriasis or benign prostate hypertrophy. Specific inventive examples for substituted camptothecins such as irinotecan and topotecan include: use for the treatment of leukemias (acute and chronic, AML, ALL, CLL, CML); use for the treatment of myelodysplastic syndrome (MDS); use for the treatment of angiogenic diseases; use for the treatment of benign prostate hypertrophy; use for the treatment of psoriasis; use for the treatment of gout; use for the treatment of autoimmune conditions; use for prevention of transplantation rejection; use for restenosis prevention in cardiovascular disease; use for the treatment of mycosis fungoides; use in bone marrow transplantation; use as an anti-infective; use for the treatment of AIDS; use for the treatment of lymphoma; use for the treatment of mantle cell lymphoma; use for the treatment of meningeal leukemia; use for the treatment of malignant meningitis; use for the treatment of cutaneous T-cell lymphoma; use for the treatment of Barrett's esophagus; use for the treatment of anaplastic gliomas; use for the treatment of triple-negative breast cancer; use for the treatment of Braf-mutated melanoma; use for the treatment of BTK-resistant CLL; use for the treatment of lymphoma; use for the treatment of chordoma; use for the treatment of Kras-mutated colon cancer; use for the treatment of pediatric tumors including brain tumors and sarcoma; use for the treatment of neuroblastoma; use for the treatment of rhabdomyosarcoma; use for the treatment of Ewing's sarcoma; use for the treatment of medulloblastoma; use for the treatment of neuroendocrine tumors; use for the treatment of diffuse intrinsic pontine glioma (DIPG); use for the treatment of colorectal cancer; use for the treatment of benign colorectal tumors; use for the treatment of ovarian cancer; use for the treatment of breast cancer; use for the treatment of superficial breast cancer; use for the treatment of chest wall recurrences; or use for the treatment of leptomeningeal disease (LMD).

### VI. Disease Stages

Improvements for suboptimal therapeutics including substituted camptothecins such as, but not limited to, irinotecan and topotecan are made by alterations in the stage of disease at diagnosis/progression that the compound is administered. General examples include: the use of chemotherapy for non-resectable local disease, prophylactic use to prevent metastatic spread or inhibit disease progression or conversion to more malignant stages. Specific inventive examples for substituted camptothecins such as irinotecan and topotecan include: use for the treatment of localized polyp stage colon cancer; use for the treatment of leukoplakia in the oral cavity; use against angiogenesis inhibition to prevent or limit metastatic spread; or use against HIV with AZT, DDI, or reverse transcriptase inhibitors.

### VII. Other Indications

Improvements for suboptimal therapeutics including substituted camptothecins such as, but not limited to, irinotecan and topotecan are made by using the compound for non-malignant diseases and conditions. General examples include: treatment of premalignant conditions; treatment of benign hyperproliferative conditions; treatment of infections; treatment of parasites; usage to relieve pain; control of pleural effusions. Specific inventive examples for substituted camptothecins such as irinotecan and topotecan include: use as anti-infectives; use as antivirals; use as antibacterials; use for pleural effusions; use as antifungals; use as anti-parasitics; use for treatment of eczema; use for treatment of shingles; use for treatment of condylomata; use as an anti HPV agent; use as an anti-HSV agent; use for treatment of early and late stage MDS (myelodysplastic syndrome); use for treatment of polycythemia vera; use for treatment of atopic dermatitis (AD); use for treatment of hand-foot syndrome; use for treatment of palmar-plantar erythrodysesthesia (PPE); or use for treatment of Stevens-Johnson syndrome (SJS).

### VIII. Patient Selection

Improvements for suboptimal therapeutics including substituted camptothecins such as, but not limited to, irinotecan and topotecan are made by alterations to the type of patient that would best tolerate or benefit from the use of the compound. General examples include: use of pediatric doses for elderly patients, altered doses for obese patients; exploitation of co-morbid disease conditions such as diabetes, cirrhosis, or other co-morbid disease or conditions that may uniquely exploit a feature of the compound. Specific inventive examples for substituted camptothecins such as irinotecan and topotecan include: patients with disease conditions with high levels of metabolic enzymes, histone deacetylase, protein kinases, or ornithine decarboxylase; patients with disease conditions with low levels of metabolic enzymes, histone deacetylase, protein kinases, or ornithine decarboxylase; patients with low or high susceptibility to thrombocytopenia or neutropenia; patients intolerant of GI toxicities; patients with over- or under-expression of jun, GPCR's and signal transduction proteins, VEGF, prostate specific genes, protein kinases, or telomerases; patients with high or low levels of activity of UDP-glucuronosyltransferase (UGT); patients with results of liquid biopsy suggesting variations in treatment; patients with results of genomic analysis suggesting variations in treatment; patients with results of proteomic analysis suggesting variations in treatment; patients with results of BRCA1 or BRCA2 gene analysis suggesting variations in treatment; patients with wild-type or methylated MGMT promoter; patients with mutations in/DH1; or patients with mutations in HER2.

### IX. Patient or Disease Phenotype

Improvements for suboptimal therapeutics including substituted camptothecins such as, but not limited to, irinotecan and topotecan are made by more precise identification of a patient's ability to tolerate, metabolize and exploit the use of the compound leading to consideration of the patient or disease phenotype. General examples include: use of diagnostic tools and kits to better characterize a patient's ability to process/metabolize a chemotherapeutic agent or their susceptibility to toxicity caused by potential specialized cellular, metabolic, or organ system phenotypes. Specific inventive examples for substituted camptothecins such as irinotecan and topotecan include: diagnostic tools, techniques, kits and assays to confirm a patient's particular phenotype and for the measurement of metabolism-associated enzymes, specific metabolites, level or expression of histone deacetylase, level or expression of protein kinases, ornithine decarboxylase, VEGF, prostate specific genes, protein kinases, telomerase, jun, or GPCRs; surrogate compound dosing; detection or analysis of circulating tumor proteins; low dose drug pre-testing for enzymatic status; upregulation of protein expression for ERBB2, GRB7, JNK1 kinase, BCL2, MK167, phospho-Akt, CD-68, or BAG1 as associated with responsiveness to treatment of colorectal cancer by irinotecan; downregulation of protein expression for Erk1 kinase, phospho-GSK-3β, MMP11, CTSL2, CCNB1, BIRC5, STK6, MRP14 and GSTM1 as associated with responsiveness to treatment of colorectal cancer by irinotecan; protein expression for AMD1, CTSC, EIF1AX, C12orf30, DDX54, PTPN2, and TBX3 as affecting therapeutic efficacy of irinotecan; expression level of topoisomerase I; activity of carboxylesterase; activity of ABC transporter genes, including genes for MRP-1, MRP-2, and ABCG2; plasma level of tissue inhibitor of metalloproteinase-1 (TIMP-1); or the level of a marker that is one or more of 5-aminoimidazole-4-carboxamide ribotide, alanine, aspartic acid, cysteine, cysteine-glutathione disulfide, glycerol-3-phosphate, histidine, isoleucine, leucine, lysine, methionine sulfoxide, N⁶,N⁶,N⁶-trimethyllysine, N⁶-acetyllysine, octanoic acid, serine, taurocholic acid, threonine, tryptophan, tyrosine, and valine.

### X. Patient or Disease Genotype

Improvements for suboptimal therapeutics including substituted camptothecins such as, but not limited to, irinotecan and topotecan are made by testing and analyzing a patient's genotype for unique features that may be of value to predict efficacy, toxicity, metabolism, or other factors affecting therapeutic efficacy or the occurrence of side effects leading to consideration of the patient or disease genotype. General examples include: biopsy samples of tumors or normal tissues (e.g., leukocytes or subclasses of leukocytes such as lymphocytes) may also be taken and analyzed to specifically tailor or monitor the use of a particular drug against a gene target, unique tumor gene expression pattern, or particular SNPs (single nucleotide polymorphisms), to enhance efficacy or to avoid particular drug-sensitive normal tissue toxicities. Specific inventive examples for substituted camptothecins such as irinotecan and topotecan include: diagnostic tools, techniques, kits and assays to confirm a patient's particular genotype; gene/protein expression chips and analysis; single nucleotide polymorphism (SNP) assessment; SNPs for histone deacetylase, ornithine decarboxylase, S-adenosyl methionine, GPCR's, protein kinases, telomerase, jun; identification and measurement of metabolism enzymes and metabolites; mutation in specific wild-type and mutated genes; epigenetics via methylation and acetylation; mutations in genes for UGT, MGMT, BRCA, IDH, He 2, EGFR; determination of expression for wild-type or mutated genes; detection or analysis of circulating tumor DNA or RNA; use of genome-wide sequencing; determination of the presence of A or G at genotypic marker −3156 of the UGT1A 1 gene or at any position in linkage equilibrium with this genotypic marker wherein A positively correlates with irinotecan toxicity and G correlates with the absence of irinotecan toxicity, such that homozygosity for A indicates increased toxicity; a genotypic marker associated with polymorphisms in the TATA box within the promoter region for the UGT1A1 gene such that the presence of 7 TA repeats in the TATA box reduces expression of UGT1A1 and predisposes to increased toxicity; occurrence of variant alleles of MRP1; existence of single nucleotide polymorphisms in a region encoding APCDD1L, R3HCC1, OR5112, MKKS, EDEM3, or ACOX1; a polymorphism that is (G/G) for rs1792689, (C/T) or (C/C) for rs2268753; (G/G) for rs17776182, (A/A) for rs7570532, or (A/G) or (G/G) for rs4946935 which is favorable for efficacy of irinotecan when administered together with bevacizumab; a polymorphism that is (G/G) for rs1792689, (C/T) or (C/C) for rs2268753, (G/G) for rs17776182, (A/A) for rs7570532, and (A/G) or (G/G) for rs4946935, which is unfavorable for efficacy of irinotecan when administered together with bevacizumab; or the occurrence of a polymorphism rs1980576 in APCDD1L which is A in the wild-type and G in the mutant and where irinotecan has the strongest therapeutic effect when the genome is homozygous for A.

### XI. Pre/Post-Treatment Preparation

Improvements for suboptimal therapeutics including, but not limited to, substituted camptothecins such as irinotecan and topotecan are made by specialized preparation of a patient prior to or after the use of a therapeutic agent. General examples include: induction or inhibition of metabolizing enzymes, specific protection of sensitive normal tissues or organ systems. Specific inventive examples for substituted camptothecins such as irinotecan and topotecan include: use of colchicine or analogs; use of diuretics such as probenecid; use of uricase; non-oral use of nicotinamide; use of sustained release forms of nicotinamide; use of inhibitors of poly-ADP ribose polymerase; use of caffeine; use of leucovorin rescue; use of infection control; use of antihypertensives; use of alteration of stem cell populations; pretreatment to limit or prevent graft-versus-host (GVH) cytokine storm reactions; use of anti-inflammatories; anaphylactic reaction suppression; or use of anti-diarrhea treatments.

### XII. Toxicity Management

Improvements for suboptimal therapeutics including substituted camptothecins such as, but not limited to, irinotecan and topotecan are made by use of additional drugs or procedures to prevent or reduce potential side effects or toxicities. General examples include: the use of anti-emetics, anti-nausea agents, hematological support agents to limit or prevent neutropenia, anemia, or thrombocytopenia, vitamins, antidepressants, treatments for sexual dysfunction, or other treatments to reduce side effects or toxicities. Specific inventive examples for substituted camptothecins such as irinotecan and topotecan include: use of colchicine or analogs; use of diuretics such as probenecid; use of uricase; non-oral use of nicotinamide; use of sustained-release forms of nicotinamide; use of inhibitors of poly-ADP-ribose polymerase; use of caffeine; leucovorin rescue; use of sustained-release allopurinol; non-oral use of allopurinol; use of bone marrow transplant stimulants, blood, platelet infusions, Neupogen, G-CSF, or GM-CSF; use of agents for pain management; use of anti-inflammatories; administration of fluids; administration of corticosteroids; administration of insulin control medications; administration of antipyretics; administration of anti-nausea treatments; administration of an anti-diarrhea treatment; administration of N-acetylcysteine; administration of antihistamines; administration of agents to limit or prevent mucositis; administration of agents to limit or prevent graft-versus-host (GVH) reactions or cytokine storm reactions; administration of antifungal agents; administration of sodium thiosulfate; administration of glutathione; use of platelet transfusions; administration of epinephrine or anti-inflammatory corticosteroids for allergic or anaphylactic reactions; administration of lidocaine or other local anesthetics; administration of vasoconstrictors; administration of vasodilators; or administration of cephalosporin antibiotics.

### XIII. Pharmacokinetic/Pharmacodynamic Monitoring

Improvements for suboptimal therapeutics including substituted camptothecins such as, but not limited to, irinotecan and topotecan are made by the use of monitoring drug levels after dosing in an effort to maximize a patient's drug plasma level, to monitor the generation of toxic metabolites, or to monitor concentrations of ancillary medicines that could be beneficial or harmful in terms of drug-drug interactions. General examples include: the monitoring of drug plasma protein binding and monitoring of drug plasma levels. Specific inventive examples for substituted camptothecins such as irinotecan and topotecan include: multiple determinations of drug plasma levels; multiple determinations of metabolites in the blood or urine; measurement of polyamines; determination of density of LAT-1 surface receptors; use of gene sequencing to determine levels of activation of specific genes; determination of levels of immune effectors; determination of level of prodrug conversion of irinotecan to SN-38; or determination of level of glucuronidation of SN-38.

### XIV. Drug Combinations

Improvements for suboptimal therapeutics including substituted camptothecins such as, but not limited to, irinotecan and topotecan are made by exploiting unique drug combinations that may provide a more than additive or synergistic improvement in efficacy or side-effect management. In some cases, the combination in the same dose form. General examples include: alkylating agents with anti-metabolites, topoisomerase inhibitors with anti-tubulin agents. Specific inventive examples for substituted camptothecins such as irinotecan and topotecan include: use with other topoisomerase inhibitors; use with fraudulent nucleosides; use with fraudulent nucleotides; use with thymidylate synthetase inhibitors; use with signal transduction inhibitors; use with cisplatin or platinum analogs; use with alkylating agents such as BCNU, Gliadel wafers, CCNU, bendamustine (Treanda), or temozolomide (Temodar); use with anti-tubulin agents; use with antimetabolites; use with berberine; use with apigenin; use with amonafide; use with colchicine or colchicine analogs; use with genistein; use with etoposide; use with cytarabine; use with vinca alkaloids; use with 5-fluorouracil; use with curcumin; use with NF-κB inhibitors; use with rosmarinic acid; use with dianhydrogalactitol; use with dibromodulcitol; use with biological therapies such as antibodies such as Avastin, Rituxan, Herceptin, Erbitux, PD-1 and PDL-1 inhibitors; use with prednimustine; use with DNA and RNA therapeutics; use with Braf inhibitors; use with BTK inhibitors; use with 5-azacytidine; use with decitabine; use with PARP inhibitors; use with hypomethylating agents; use with histone deacetylase inhibitors; use with vincristine; use with thalidomide; use with leucovorin; use with trifluridine; use with tipiracil hydrochloride; use with aflibercept; use with folinic acid; use with oxaliplatin; use with 5-(5-(2-(3-aminopropoxy)-6-methoxyphenyl)-1H-pyrazol-3-ylamino)pyrazine-2-carbonitrile; use with EGFR inhibitors; use with VEGF inhibitors; use with a humanized anti-EGFR IgG1 antibody; use with 4-iodo-3-nitrobenzamide or metabolites thereof; use with bevacizumab; use with immunotherapies including: antibodies binding to alpha-PDL1, alpha-44BB, alpha-CTLA4, or alpha-OX40; or atezolizumab, avelimumab, nivolumab, pembrolizumab, ipilimumab, tremelimumab, or durvalumab; Chk1-directed therapeutic agents such as prexasertib; topoisomerase 2-directed therapeutic agents such as aldozurubicin; DNA inhibitors such as lurbinectedin; and Notch ADC-modulating agents such as rovalpituzumab tesirine; use with dilpacimab; or use with an MRP inhibitor such as valspodar (SDZ-PSC 833), tert-butyl 2-[(3S,6S,9S,15S,21S,24S,27S,30S)-15,18-bis[(2S)-butan-2-yl]-6-[(4-methoxyphenyl)methyl]-3,10,16,19,22,28-hexamethyl-2,5,8,11,14,17,20,23,26,29-decaoxo-9,24,27-tri(propan-2-yl)-4-oxa-1,7,10,13,16,19,22,25,28-nonazabicyclo[28.4.0]tetratriacontan-21-yl]acetate (SDZ 280-446), sodium 3-[[3-[(E)-2-(7-chloroquinolin-2-yl)ethenyl]phenyl]-[3-(dimethylamino)-3-oxopropyl]sulfanylmethyl]sulfanylpropanoate (MK571), dofequidar (MS209), 2-(4-benzhydrylpiperazin-1-yl)ethyl 5-[(4R,6R)-4,6-dimethyl-2-oxo-1,3,2λ-5-dioxaphosphinan-2-yl]-2,6-dimethyl-4-(3-nitrophenyl)pyridine-3-carboxylate (PAK-104p), verapamil, benzbromarone, dipyridamole, furosemide, gamma-GS(naphthyl)cysteinyl-glycine diethyl ester, genistein, quinidine, rifampicin, mifepristone (RU-486), or sulfinpyrazone.

### XV. Chemosensitization

Improvements for suboptimal therapeutics including substituted camptothecins such as, but not limited to, irinotecan and topotecan are made by exploiting them as chemosensitizers where no measurable activity is observed when used alone but in combination with other therapeutics a more than additive or synergistic improvement in efficacy is observed. General examples include: misonidazole with alkylating agents, tirapazamine with cisplatin. Specific inventive examples for substituted camptothecins such as irinotecan and topotecan include: as a chemosensitizer in combination with topoisomerase inhibitors; as a chemosensitizer in combination with fraudulent nucleosides; as a chemosensitizer in combination with fraudulent nucleotides; as a chemosensitizer in combination with thymidylate synthetase inhibitors; as a chemosensitizer in combination with signal transduction inhibitors; as a chemosensitizer in combination with cisplatin or platinum analogs; as a chemosensitizer in combination with alkylating agents such as BCNU, Gliadel wafers, CCNU, bendamustine (Treanda), or temozolomide (Temodar); as a chemosensitizer in combination with anti-tubulin agents; as a chemosensitizer in combination with antimetabolites; as a chemosensitizer in combination with berberine; as a chemosensitizer in combination with apigenin; as a chemosensitizer in combination with amonafide; as a chemosensitizer in combination with colchicine or analogs of colchicine; as a chemosensitizer in combination with genistein; as a chemosensitizer in combination with etoposide; as a chemosensitizer in combination with cytarabine; as a chemosensitizer in combination with vinca alkaloids; as a chemosensitizer in combination with 5-fluorouracil; as a chemosensitizer in combination with curcumin; as a chemosensitizer in combination with NF-κB inhibitors; as a chemosensitizer in combination with rosmarinic acid; as a chemosensitizer in combination with dianhydrogalactitol; as a chemosensitizer in combination with dibromodulcitol; as a chemosensitizer in combination with biological therapies such as antibodies such as Avastin, Rituxan, Herceptin, Erbitux, PD-1 and PDL-1 inhibitors; as a chemosensitizer in combination with prednimustine; as a chemosensitizer in combination with DNA and RNA therapeutics; as a chemosensitizer in combination with Braf inhibitors; as a chemosensitizer in combination with BTK inhibitors; as a chemosensitizer in combination with 5-azacytidine; as a chemosensitizer in combination with decitabine; as a chemosensitizer in combination with PARP inhibitors; as a chemosensitizer in combination with hypomethylating agents; as a chemosensitizer in combination with histone deacetylase inhibitors; or as a chemosensitizer in combination with vincristine.

### XVI. Chemopotentiation

Improvements for suboptimal therapeutics including substituted camptothecins such as, but not limited to, irinotecan and topotecan are made by exploiting them as chemopotentiators where minimal therapeutic activity is observed alone but in combination with other therapeutics a more than additive or synergistic improvement in efficacy is observed. General examples include: amonafide with cisplatin or 5-fluorouracil. Specific inventive examples for substituted camptothecins such as irinotecan and topotecan include: as a chemopotentiator in combination with topoisomerase inhibitors; as a chemopotentiator in combination with fraudulent nucleosides; as a chemopotentiator in combination with fraudulent nucleotides; as a chemopotentiator in combination with thymidylate synthetase inhibitors; as a chemopotentiator in combination with signal transduction inhibitors; as a chemopotentiator in combination with cisplatin or platinum analogs; as a chemopotentiator in combination with alkylating agents such as BCNU, Gliadel wafers, CCNU, bendamustine (Treanda), or temozolomide (Temodar); as a chemopotentiator in combination with anti-tubulin agents; as a chemopotentiator in combination with antimetabolites; as a chemopotentiator in combination with berberine; as a chemopotentiator in combination with apigenin; as a chemopotentiator in combination with amonafide; as a chemopotentiator in combination with colchicine or analogs of colchicine; as a chemopotentiator in combination with genistein; as a chemopotentiator in combination with etoposide; as a chemopotentiator in combination with cytarabine; as a chemopotentiator in combination with vinca alkaloids; as a chemopotentiator in combination with 5-fluorouracil; as a chemopotentiator in combination with curcumin; NF-κB inhibitors; as a chemopotentiator in combination with rosmarinic acid; as a chemopotentiator in combination with dianhydrogalactitol; as a chemopotentiator in combination with dibromodulcitol; as a chemopotentiator in combination with in combination with biological therapies such as antibodies such as Avastin, Rituxan, Herceptin, Erbitux, PD-1 and PDL-1 inhibitors; as a chemopotentiator in combination with prednimustine; as a chemopotentiator in combination with DNA and RNA therapeutics; as a chemopotentiator in combination with Braf inhibitors; as a chemopotentiator in combination with BTK inhibitors; as a chemopotentiator in combination with 5-azacytidine; as a chemopotentiator in combination with decitabine; as a chemopotentiator in combination with PARP inhibitors; as a chemopotentiator in combination with hypomethylating agents; as a chemopotentiator in combination with histone deacetylase inhibitors; or as a chemopotentiator in combination with vincristine.

### XVII. Post-Treatment Patient Management

Improvements for suboptimal therapeutics including substituted camptothecins such as, but not limited to, irinotecan and topotecan are made by drugs, treatments and diagnostics to allow for the maximum benefit to patients treated with a compound. General examples include: pain management, nutritional support, anti-emetics, anti-nausea therapies, anti-anemia therapy, anti-inflammatories. Specific inventive examples for substituted camptothecins such as irinotecan and topotecan include: use with therapies associated with pain management; nutritional support; anti-emetics; anti-nausea therapies; anti-anemia therapy; anti-inflammatories; antipyretics; immune stimulants; anti diarrhea medicines; famotidine; antihistamines; suppository lubricants; soothing agents; lidocaine; hydrocortisone.

### XVIII. Alternative Medicine/Therapeutic Support

Improvements for suboptimal therapeutics including substituted camptothecins such as, but not limited to, irinotecan and topotecan are made by the use of non-conventional therapeutics or methods to enhance effectiveness or reduce side effects. General examples include herbal medications and extracts. Specific inventive examples for substituted camptothecins such as irinotecan and topotecan include: herbal medications created either synthetically or through extraction including NF-κB inhibitors (such as parthenolide, curcumin, rosmarinic acid); natural anti-inflammatories (including rhein, parthenolide); immunostimulants (such as those found in Echinacea); antimicrobials (such as berberine); or flavonoids and flavones (such as apigenin, genistein).

### XIX. Bulk Drug Product Improvements

Improvements for suboptimal therapeutics including substituted camptothecins such as, but not limited to, irinotecan and topotecan are made by alterations in the pharmaceutical bulk substance. General examples include: salt formation, homogenous crystalline structure, pure isomers. Specific inventive examples for substituted camptothecins such as irinotecan and topotecan include: salt formation; homogenous crystalline structure; pure isomers, such as stereoisomers; increased purity; lower residual solvents; or lower residual heavy metals.

### XX. Diluent Systems

Improvements for suboptimal therapeutics including substituted camptothecins such as, but not limited to, irinotecan and topotecan are made by alterations in the diluents used to solubilize and deliver/present the compound for administration. General examples include: Cremophor-EL, cyclodextrins for poorly water-soluble compounds. Specific inventive examples for substituted camptothecins such as irinotecan and topotecan include: emulsions; dimethyl sulfoxide (DMSO); N-methyl formamide (NMF); dimethylformamide (DMF); dimethylacetamide (DMA); ethanol; benzyl alcohol; dextrose-containing water for injection; Cremophor; cyclodextrins; PEG; agents to sweeten such as saccharin, sucralose, aspartame; agents to thicken an oral dosage form such as glycerin; taste-masking effectors such as menthol, rum flavor fruit flavorings, or chocolate; or buffers to yield a pH value as buffered of less than 4.

### XXI. Solvent Systems

Improvements for suboptimal therapeutics including substituted camptothecins such as, but not limited to, irinotecan and topotecan are made by alterations in the solvents used or required to solubilize a compound for administration or for further dilution. General examples include: ethanol, dimethylacetamide (DMA). Specific inventive examples for substituted camptothecins such as irinotecan and topotecan include: emulsions; DMSO; NMF; DMF; DMA; ethanol; benzyl alcohol; dextrose-containing water for injection; Cremophor; PEG; glycerin; or cocoa butter for suppositories.

### XXII. Excipients

Improvements for suboptimal therapeutics including substituted camptothecins such as, but not limited to, irinotecan and topotecan are made by alterations in the materials/excipients, buffering agents, preservatives required to stabilize and present a chemical compound for proper administration. General examples include: mannitol, albumin, EDTA, sodium bisulfite, benzyl alcohol. Specific inventive examples for substituted camptothecins such as irinotecan and topotecan include: mannitol; albumin; EDTA; sodium bisulfite; benzyl alcohol; carbonate buffers; phosphate buffers; benzoate preservatives; glycerin; sweeteners; taste-masking agents such as rum flavor; menthol substituted celluloses; sodium azide as a preservative; or flavors for oral dosage forms.

### XXIII. Dosage Forms

Improvements for suboptimal therapeutics including substituted camptothecins such as, but not limited to, irinotecan and topotecan are made by alterations in the potential dosage forms of the compound dependent on the route of administration, duration of effect, plasma levels required, exposure to normal tissues which may induce side effects, and exposure to metabolizing enzymes. General examples include: tablets, capsules, topical gels, creams, patches, solutions, suspensions, emulsions, or suppositories. Specific inventive examples for substituted camptothecins such as irinotecan and topotecan include: liquid in gel capsules; tablets; capsules; topical gels; topical creams; patches; suppositories; lyophilized dosage fills; suppositories with quick release (<15 minutes) or long melt times (>15 minutes) leading to extended release time; temperature-adjusted suppositories; oral solutions; or suspensions of varying concentrations of active therapeutic agent or prodrug, such as 1-100 mg/mL.

### XXIV. Dosage Kits and Packaging

Improvements for suboptimal therapeutics including substituted camptothecins such as, but not limited to, irinotecan and topotecan are made by alterations in the dosage forms, container/closure systems, accuracy of mixing and dosage preparation and presentation. General examples include: amber vials to protect from light, stoppers with specialized coatings. Specific inventive examples for substituted camptothecins such as irinotecan and topotecan include: amber vials to protect from light; stoppers with specialized coatings to improve shelf-life stability; specialized dropper measuring devices; single-use or multiple-use container closure systems; dosage forms suitable for testing for allergies; suppository delivery devices; epinephrine pens for side effect management; physician and nurse assistance gloves; measuring devices; metered syringes; dosage cups configured to deliver defined doses; or two-component oral solution systems where therapeutic is added to an oral diluent.

### XXV. Drug Delivery Systems

Improvements for suboptimal therapeutics including substituted camptothecins such as, but not limited to, irinotecan and topotecan are made by the use of delivery systems to improve the potential attributes of a pharmaceutical product such as convenience, duration of effect, or reduction of side effects or toxicities. General examples include: nanocrystals, bioerodible polymers, liposomes, slow release injectable gels, microspheres. Specific inventive examples for substituted camptothecins such as irinotecan and topotecan include: nanocrystals; bioerodible polymers; liposomes; slow-release injectable gels; microspheres; suspensions with glycerin; meltable drug release suppositories with polymers such as cocoa butter alone or in combination with PEG, lecithin, or polylactide/polyglycolide; rectal plugs for drug delivery; micro- or nano-emulsions; cyclodextrins; or topical delivery systems.

### XXVI. Drug Conjugate Forms

Improvements for suboptimal therapeutics including substituted camptothecins such as, but not limited to, irinotecan and topotecan are made by alterations to the parent molecule with covalent, ionic, or hydrogen-bonded moieties to alter the efficacy, toxicity, pharmacokinetics, metabolism, or route of administration. General examples include: polymer systems such as polyethylene glycols, polylactides, polyglycolides, amino acids, peptides, multivalent linkers. Specific inventive examples for substituted camptothecins such as irinotecan and topotecan include: polyethylene glycols; polylactides; polyglycolides; amino acids; peptides; or multivalent linkers.

### XXVII. Compound Analogs

Improvements for suboptimal therapeutics including substituted camptothecins such as, but not limited to, irinotecan and topotecan are made by alterations to the parent structure of a molecule with additional chemical functionalities that may alter efficacy, or reduce toxicity, pharmacological performance, optimum route of administration, or other factors associated with the therapeutic activity or administration of the molecule. General examples include: alteration of side chains to increase or decrease lipophilicity, additional chemical functionalities to alter reactivity, electron affinity, or binding capacity, or the preparation of salt forms. Specific inventive examples for substituted camptothecins such as irinotecan and topotecan include: alteration of side chains to increase or decrease lipophilicity; additional chemical functionalities to alter reactivity, electron affinity, or binding capacity; or preparation of salt forms.

### XXVIII. Prodrug Systems

Improvements for suboptimal therapeutics including substituted camptothecins such as, but not limited to, irinotecan and topotecan are made by alterations to the molecule such that improved pharmaceutical performance is gained with a variant of the active molecule in that after introduction into the body a portion of the molecule is cleaved to reveal the preferred active molecule. General examples include: enzyme sensitive esters, dimers, Schiff bases. Specific inventive examples for substituted camptothecins such as irinotecan and topotecan include: enzyme sensitive esters; dimers; Schiff bases; pyridoxal complexes; caffeine complexes; gastrointestinal system transporters; or permeation enhancers.

### XXIX. Multiple Drug Systems

Improvements for suboptimal therapeutics including substituted camptothecins such as, but not limited to, irinotecan and topotecan are made by the use of additional compounds, biological agents that when administered in the proper fashion, a unique and beneficial effect can be realized. General examples include: inhibitors of multi-drug resistance, specific drug resistance inhibitors, specific inhibitors of selective enzymes, signal transduction inhibitors, repair inhibition. Specific inventive examples for substituted camptothecins such as irinotecan and topotecan include: inhibitors of multi-drug resistance; specific drug resistance inhibitors; specific inhibitors of selective enzymes; signal transduction inhibitors; repair inhibition; topoisomerase inhibitors with non-overlapping side effects; multiple agents with different therapeutic mechanisms as in MIME chemotherapy for Hodgkin's disease; temozolomide; substituted hexitols; cephalosporin antibiotics such as cefixime; caffeine; or PARP inhibitors.

### XXX. Biotherapeutic Enhancement

Improvements for suboptimal therapeutics including substituted camptothecins such as, but not limited to, irinotecan and topotecan are made by its use in combination as sensitizers/potentiators with biological response modifiers. General examples include: use in combination as sensitizers/potentiators with biological response modifiers, cytokines, lymphokines, therapeutic antibodies, antisense therapies, gene therapies. Specific inventive examples for substituted camptothecins such as irinotecan and topotecan include: cytokines; lymphokines; therapeutic antibodies such as Avastin, Herceptin, Rituxan, and Erbitux; antisense therapies; gene therapies; ribozymes; RNA interference; or cell-based therapeutics such as CAR-T.

### XXXI. Biotherapeutic Resistance Modulation

Improvements for suboptimal therapeutics including substituted camptothecins such as, but not limited to, irinotecan and topotecan are made by exploiting their selective use to overcome developing or complete resistance to the efficient use of biotherapeutics. General examples include: tumors resistant to the effects of biological response modifiers, cytokines, lymphokines, therapeutic antibodies, antisense therapies, gene therapies. Specific inventive examples for substituted camptothecins such as irinotecan and topotecan include: the use against tumors resistant to the effects of biological response modifiers, cytokines, lymphokines, or therapeutic antibodies such as Avastin, Rituxan, Herceptin, Erbitux; the use against tumors resistant to the effects of antisense therapies; the use against tumors resistant to the effects of gene therapies; the use against tumors resistant to the effects of ribozymes; the use against tumors resistant to RNA interference; or the use against tumors resistant to CAR-T therapy.

### XXXII. Radiation Therapy Enhancement

Improvements for suboptimal therapeutics including substituted camptothecins such as, but not limited to, irinotecan and topotecan are made by exploiting their use in combination with ionizing radiation, phototherapies, heat therapies, radio-frequency generated therapies. General examples include: hypoxic cell sensitizers, radiation sensitizers/protectors, photosensitizers, radiation repair inhibitors. Specific inventive examples for substituted camptothecins such as irinotecan and topotecan include: use with hypoxic cell sensitizers; use with radiation sensitizers/protectors; use with photosensitizers; use with radiation repair inhibitors; use with agents for thiol depletion; use with vaso-targeted agents; use with radioactive seeds; use with radionuclides; use with radiolabeled antibodies; or use with brachytherapy.

### XXXIII. Novel Mechanisms of Action

Improvements for suboptimal therapeutics including substituted camptothecins such as, but not limited to, irinotecan and topotecan are made by optimizing their utility by determining the various mechanisms of actions, biological targets of a compound for greater understanding and precision to better exploit the utility of the molecule. General examples include: imatinib (Gleevec) for chronic myelocytic leukemia (CML), arsenic trioxide for acute promyelocytic leukemia (APL), retinoic acid for APL. Specific inventive examples for substituted camptothecins such as irinotecan and topotecan include: inhibitors of poly-ADP ribose polymerase (PARP); agents that affect vasculature; agents that affect vasodilation; oncogenic targeted agents; signal transduction inhibitors; EGFR inhibitors; protein kinase C inhibitors; phospholipase C downregulating agents; jun downregulating agents; downregulating agents for histone genes, downregulating agents for VEGF, agents that modulate the activity of ornithine decarboxylase; agents that modulate the activity of jun D; agents that modulate the activity of v-jun; agents that modulate the activity of GPCRs; agents that modulate the activity of protein kinase A; agents that modulate the activity of telomerase; agents that modulate the activity of prostate specific genes; agents that modulate the activity of protein kinases; or agents that modulate the activity of histone deacetylase.

### XXXIV. Selective Target Cell Population Therapeutics

Improvements for suboptimal therapeutics including substituted camptothecins such as, but not limited to, irinotecan and topotecan are made by more precise identification and exposure of the compound to those select cell populations where the compounds effect can be maximally exploited. General examples include: tirapazamine and mitomycin c for hypoxic cells, vinca alkaloids for cells entering mitosis. Specific inventive examples for substituted camptothecins such as irinotecan and topotecan include: use against radiation sensitive cells; use against radiation resistant cells; use against energy depleted cells; or use against endothelial cells.

### XXV. Use of Liposomal Formulations for Administration

Improvements for suboptimal therapeutics including substituted camptothecins such as, but not limited to, irinotecan and topotecan are made by use of liposomal formulations for delivery of irinotecan, topotecan, or derivatives or analogs of irinotecan or topotecan. The liposomal formulations can include cardiolipin, phospholipids such as phosphatidylcholine, α-tocopherol, cholesterol, or other components such as polyethylene glycol. The liposomes can be unilamellar or bilamellar. The liposomes can also include substituted ammonium compounds or substituted sugars.

### XXVI. Use of Crystalline Polymorphs

Improvements for suboptimal therapeutics including substituted camptothecins such as, but not limited to, irinotecan and topotecan are made by use of crystalline polymorphs that can improve bioavailability and therapeutic efficacy. Polymorphism is the property of molecules, including many small-molecule therapeutic agents, to adopt more than one crystalline form in the solid state. The crystalline form adopted by the molecule is typically determined by the particular crystallization process employed, including variables such as the solvent used, the inclusion of an anti-solvent, and the temperature employed. A single molecule can give rise to a variety of solids having distinct physical properties that can be measured in a laboratory like its thermal behavior, melting point and differential scanning calorimetry (“DSC”) thermogram, dissolution rate, flowability, X-ray diffraction pattern, infrared absorption spectrum, including the infrared diffuse-reflectance pattern, and NMR spectrum. The differences in the physical properties of polymorphs result from the orientation and intermolecular interactions of adjacent molecules in the bulk solid. Accordingly, polymorphs are distinct solids sharing the same molecular formula which can yet have distinct advantageous and/or disadvantageous physical properties compared to other forms in the polymorph family. One property of a pharmaceutical compound that can vary depending upon its polymorphic form is its rate of dissolution in aqueous solvent. The rate of dissolution can have therapeutic consequences since it can affect the rate that an orally administered pharmaceutical is delivered to the bloodstream of a patient. Other properties of a pharmaceutical compound that can vary depending upon its polymorphic form include properties such as flowability and tabletability.

### XXVII. Use of Stereoisomers

Improvements for suboptimal therapeutics including substituted camptothecins such as, but not limited to, irinotecan and topotecan are made by use of stereoisomers of these therapeutic agents that can improve bioavailability and therapeutic efficacy. In particular, irinotecan is a chiral compound with an asymmetric carbon atom, leading to enantiomeric forms. Topotecan, which is a derivative of irinotecan, is also a chiral compound with an asymmetric carbon atom, leading to enantiomeric forms. Substituents present in derivatives or analogs of irinotecan or topotecan can also introduce chiral carbons or other sources of asymmetry, leading to the occurrence of enantiomeric or diastereomeric forms. Stereoisomeric forms can be, but are not limited to, specific enantiomers, racemates, or preparations enhanced in one specific isomer, such as preparations comprising 60%, 65%, 70%, 75%, 80%, 85%, 90%, 95%, 96%, 97%, 98%, or 99% of a specific enantiomer.

When the irinotecan, topotecan, or derivative or analog of irinotecan or topotecan is used to treat a malignancy, the malignancy can be, but is not limited to, colorectal cancer (including colon cancer), pancreatic cancer, lung cancer (including small-cell lung cancer and non-small-cell lung cancer), breast cancer, gastric cancer (including gastroesophageal cancer), locally advanced or metastatic breast cancer, ovarian cancer, rhabdomyosarcoma, cervical cancer, neuroblastoma, glioblastoma multiforme, Ewing's sarcoma, non-Hodgkin's lymphoma, endometrial cancer, and oligodendroglioma. In particular, irinotecan can be used to treat colon cancer or pancreatic cancer. In particular, topotecan can be used to treat ovarian cancer, cervical cancer, and small-cell lung cancer.

Methods and compositions according to the present invention can alternatively be used to treat other malignancies, including, but not limited to, human sarcomas and carcinomas. These malignancies include, but are not limited to: fibrosarcoma; myxosarcoma; liposarcoma, chondrosarcoma; osteogenic sarcoma; chordoma; angiosarcoma; endotheliosarcoma; lymphangiosarcoma; lymphangioendotheliosarcoma; synovioma; mesothelioma; leiomyosarcoma; rhabdomyosarcoma; Kras-mutated colon carcinoma; anal carcinoma; esophageal cancer; hepatocellular cancer; bladder cancer; endometrial cancer; pancreatic cancer; triple-negative breast cancer; prostate cancer; atrial myxomas; squamous cell carcinoma; basal cell carcinoma; adenocarcinoma; sweat gland carcinoma; sebaceous gland carcinoma; thyroid and parathyroid neoplasms; papillary carcinoma; papillary adenocarcinoma; cystadenocarcinoma; medullary carcinoma; bronchogenic carcinoma; renal cell carcinoma; hepatoma; bile duct carcinoma; choriocarcinoma; seminoma; embryonal carcinoma; testicular tumor; bladder carcinoma; epithelial carcinoma; glioma; pituitary neoplasms; astrocytoma; medulloblastoma; craniopharyngioma; ependymoma; pinealoma; hemangioblastoma; acoustic neuroma; schwannoma; oligodendroglioma; meningioma; spinal cord tumors; melanoma, including Braf-mutated melanoma; neuroblastoma; pheochromocytoma; endocrine neoplasia, Types 1-3; retinoblastoma; leukemias, including acute lymphocytic leukemia and acute myelocytic leukemia (including myeloblastic, promyelocytic, myelomonocytic, monocytic, and erythroleukemia), chronic leukemia (including chronic myelocytic (granulocytic) leukemia and chronic lymphocytic leukemia, including BTK-resistant chronic lymphocytic leukemia), and meningeal leukemia; polycythemia vera; lymphoma, including Hodgkin's lymphoma, non-Hodgkin's lymphoma, mantle cell lymphoma, and cutaneous T-cell lymphoma; multiple myeloma; Waldenström's macroglobulinemia; mycosis fungoides; leptomeningeal cancer; pediatric brain tumors; pediatric sarcoma; ovarian osteogenic sarcoma; small-cell carcinoma of the ovary, including the hypercalcemic type, and heavy chain disease.

In the inventive compositions and methods, the term suboptimal therapy includes agents where Phase I toxicity precluded further human clinical evaluation. It also includes those agents from Phase II trials where limited (e.g., <25% response rates) or no significant treatment responses were identified. Also, suboptimal therapy includes those agents, the subject of Phase III clinical trials the outcome of which was either medically or statistically not significant to warrant regulatory submission or approval by government agencies for commercialization for commercialized agents whose clinical performance (i.e. response rates) as a monotherapy are less than 25%, or whose side effects are severe enough to limit wide utility. Agents with suboptimal clinical activity include but are not limited to the following: small chemical therapeutics, natural products, biologics such as peptides, protein antibody drug conjugates, or vaccines, including cell based therapies. More specifically, methods and compositions according to the present invention include methods and composition that include irinotecan, topotecan, and derivatives and analogs thereof. Suitable derivatives and analogs of irinotecan or topotecan are as described above.

One aspect of the present invention is a method to improve the efficacy and/or reduce the side effects of the administration of irinotecan, topotecan, or a derivative or analog of irinotecan or topotecan for treatment of benign or neoplastic hyperproliferative diseases, infections, inflammatory disease or conditions, or immunological diseases or conditions comprising the steps of:
(1) identifying at least one factor or parameter associated with the efficacy and/or occurrence of side effects of the administration of the irinotecan, topotecan, or the derivative or analog of irinotecan or topotecan for the treatment of benign or neoplastic hyperproliferative diseases, infections, inflammatory disease or conditions, or immunological diseases; and
(2) modifying the factor or parameter to improve the efficacy and/or reduce the side effects of the administration of the irinotecan, topotecan, or the derivative or analog of irinotecan or topotecan for the treatment of benign or neoplastic hyperproliferative diseases, infections, inflammatory disease or conditions, or immunological diseases.

Typically, the factor or parameter is selected from the group consisting of:
(1) dose modification;
(2) route of administration;
(3) schedule of administration;
(4) indications for use;
(5) disease stages;
(6) other indications;
(7) patient selection;
(8) patient or disease phenotype;
(9) patient or disease genotype;
(10) pre-post/treatment preparation
(11) toxicity management;
(12) pharmacokinetic/pharmacodynamic monitoring;
(13) drug combinations;
(14) chemosensitization;
(15) chemopotentiation;
(16) post-treatment management;
(17) alternative medicine/therapeutic support;
(18) bulk drug product improvements;
(19) diluent systems;
(20) solvent systems;
(21) excipients;
(22) dosage forms;
(23) dosage kits and packaging;
(24) drug delivery systems;
(25) drug conjugate forms;
(26) compound analogs;
(27) prodrug systems;
(28) multiple drug systems;
(29) biotherapeutic enhancement;
(30) biotherapeutic resistance modulation;
(31) radiation therapy enhancement;
(32) novel mechanisms of action;
(33) selective target cell population therapeutics;
(34) use of liposomes for drug delivery;
(35) use of crystalline polymorphisms; and
(36) use of stereoisomers.

When the improvement made is by dose modification, the dose modification can be, but is not limited to, at least one dose modification selected from the group consisting of:
(a) intravenous infusion for hours to days;
(b) biweekly, tri-weekly, or monthly administration;
(c) doses greater than 100 mg/m²/day;
(d) progressive escalation of dosing from 100 mg/m²/day based on patient tolerance;
(e) doses less than 2 mg/m² for greater than 14 days;
(f) dose modification associated with use of polyamine to modulate metabolism;
(g) dose modification associated with use of eflornithine to modulate metabolism;
(h) selected and intermittent boost dose administration;
(i) bolus single and multiple doses escalating from 100 mg/m²;
(j) oral doses below 30 or above 130 mg/m²;
(k) low potency (1-10 mg/mL) oral solutions or suspensions; and
(l) medium potency (10-200 mg/mL) oral solutions or suspensions.

Polyamines include, but are not limited to, putrescene, spermidine and spermine.

Eflornithine, which occurs in two enantiomeric forms, is a structural analog of the amino acid L-ornithine and is an irreversible inhibitor of the enzyme ornithine decarboxylase (ODC).

When the improvement is made by route of administration, the route of administration can be, but is not limited to, at least one route of administration selected from the group consisting of:
(a) topical administration;
(b) intravesicular administration for bladder cancer;
(c) oral administration;
(d) slow release oral delivery;
(e) intrathecal administration;
(f) intraarterial administration;
(g) continuous infusion;
(h) intermittent infusion;
(i) administration by use of large-volume oral solutions;
(j) buccal administration; and
(k) rectal administration.

When the improvement is made by schedule of administration, the schedule of administration can be, but is not limited to, at least one schedule of administration selected from the group consisting of:
(a) daily administration;
(b) weekly administration for three weeks;
(c) weekly administration for two weeks;
(d) biweekly administration;
(e) biweekly administration for three weeks with a 1-2 week rest period;
(f) intermittent boost dose administration;
(g) administration daily for one week then once per week for multiple weeks; and
(h) administration daily on days 1-5, 8-12 every three weeks, 2-5 times per day.

When the improvement is made by indications for use, the indication for use can be, but is not limited to, at least one indication for use selected from the group consisting of:
(a) use for the treatment of leukemias, including acute and chronic leukemias, including AML, ALL, CLL, CML;
(b) use for the treatment of myelodysplastic syndrome (MDS);
(c) use for the treatment of angiogenic diseases;
(d) use for the treatment of benign prostate hypertrophy;
(e) use for the treatment of psoriasis;
(f) use for the treatment of gout;
(g) use for the treatment of autoimmune conditions;
(h) use for prevention of transplantation rejection;
(i) use for restenosis prevention in cardiovascular disease;
(j) use for the treatment of mycosis fungoides;
(k) use in bone marrow transplantation;
(l) use as an anti-infective;
(m) use for the treatment of AIDS;
(n) use for the treatment of lymphoma;
(o) use for the treatment of mantle cell lymphoma;
(p) use for the treatment of meningeal leukemia;
(q) use for the treatment of malignant meningitis;
(r) use for the treatment of cutaneous T-cell lymphoma;
(s) use for the treatment of Barrett's esophagus;
(t) use for the treatment of anaplastic gliomas;
(u) use for the treatment of triple-negative breast cancer;
(v) use for the treatment of Braf-mutated melanoma;
(w) use for the treatment of BTK-resistant CLL;
(x) use for the treatment of lymphoma;
(y) use for the treatment of chordoma;
(z) use for the treatment of Kras-mutated colon cancer;
(aa) use for the treatment of pediatric tumors including brain tumors and sarcoma;
(ab) use for the treatment of neuroblastoma;
(ac) use for the treatment of rhabdomyosarcoma;
(ad) use for the treatment of Ewing's sarcoma;
(ae) use for the treatment of medulloblastoma;
(af) use for the treatment of neuroendocrine tumors;
(ag) use for the treatment of diffuse intrinsic pontine glioma (DIPG);
(ah) use for the treatment of colorectal cancer;
(ai) use for the treatment of benign colorectal tumors;
(aj) use for the treatment of ovarian cancer;
(ak) use for the treatment of breast cancer;
(al) use for the treatment of superficial breast cancer;
(am) use for the treatment of chest wall recurrences; and
(an) use for the treatment of leptomeningeal disease (LMD).

When the improvement is made by disease stage, the disease stage can be, but is not limited to, at least one disease stage selected from the group consisting of:
(a) use for the treatment of localized polyp stage colon cancer;
(b) use for the treatment of leukoplakia in the oral cavity;
(c) use against angiogenesis inhibition to prevent or limit metastatic spread; and
(d) use against HIV with AZT, DDI, or reverse transcriptase inhibitors.

When the improvement is made by other indications, the other indication can be, but is not limited to, at least one other indication selected from the group consisting of:
(a) use as anti-infectives;
(b) use as antivirals;
(c) use as antibacterials;
(d) use for pleural effusions;
(e) use as antifungals;
(f) use as anti-parasitics;
(g) use for treatment of eczema;
(h) use for treatment of shingles;
(i) use for treatment of condylomata;
(j) use as an anti HPV agent;
(k) use as an anti-HSV agent;
(l) use for treatment of early and late stage MDS (myelodysplastic syndrome);
(m) use for treatment of polycythemia vera;
(n) use for treatment of atopic dermatitis (AD);
(o) use for treatment of hand-foot syndrome;
(p) use for treatment of palmar-plantar erythrodysesthesia (PPE); and
(q) use for treatment of Stevens-Johnson syndrome (SJS).

When the improvement is made by patient selection, the patient selection can be, but is not limited to, a patient selection selected from the group consisting of:
(a) patients with disease conditions with high levels of metabolic enzymes, histone deacetylase, protein kinases, or ornithine decarboxylase;
(b) patients with disease conditions with low levels of metabolic enzymes, histone deacetylase, protein kinases, or ornithine decarboxylase;
(c) patients with low or high susceptibility to thrombocytopenia or neutropenia;
(d) patients intolerant of GI toxicities;
(e) patients with over- or under-expression of jun, GPCR's and signal transduction proteins, VEGF, prostate specific genes, protein kinases, or telomerases;
(f) patients with high or low levels of activity of UDP-glucuronosyltransferase (UGT);
(g) patients with results of liquid biopsy suggesting variations in treatment;
(h) patients with results of genomic analysis suggesting variations in treatment,
   (i) patients with results of proteomic analysis suggesting variations in treatment;
(j) patients with results of BRCA1 or BRCA2 gene analysis suggesting variations in treatment;
(k) patients with wild-type or methylated MGMT promoter;
(l) patients with mutations in/DH1; and
(m) patients with mutations in HER2.

When the improvement is made by consideration of patient or disease phenotype, the consideration of patient or disease phenotype can be, but is not limited to:
(a) diagnostic tools, techniques, kits and assays to confirm a patient's particular phenotype and for the measurement of metabolism-associated enzymes, specific metabolites, level or expression of histone deacetylase, level or expression of protein kinases, ornithine decarboxylase, VEGF, prostate specific genes, protein kinases, telomerase, jun, or GPCR's;
(b) surrogate compound dosing;
(c) detection or analysis of circulating tumor proteins;
(d) low dose drug pre-testing for enzymatic status;
(e) upregulation of protein expression for ERBB2, GRB7, JNK1 kinase, BCL2, MK167, phospho-Akt, CD-68, or BAG1 as associated with responsiveness to treatment of colorectal cancer by irinotecan;
(f) downregulation of protein expression for Erk1 kinase, phospho-GSK-3β, MMP11, CTSL2, CCNB1, BIRC5, STK6, MRP14 and GSTM1 as associated with responsiveness to treatment of colorectal cancer by irinotecan;
(g) protein expression for AMD1, CTSC, EIF1AX, C12orf30, DDX54, PTPN2, and TBX3 as affecting therapeutic efficacy of irinotecan;
(h) expression level of topoisomerase I;
(i) activity of carboxylesterase;
(j) activity of ABC transporter genes, including genes for MRP-1, MRP-2, and ABCG2;
(k) plasma level of tissue inhibitor of metalloproteinase-1 (TIMP-1); and
(l) the level of a marker that is one or more of 5-aminoimidazole-4-carboxamide ribotide, alanine, aspartic acid, cysteine, cysteine-glutathione disulfide, glycerol-3-phosphate, histidine, isoleucine, leucine, lysine, methionine sulfoxide, N⁶,N⁶,N⁶-trimethyllysine, N⁶-acetyllysine, octanoic acid, serine, taurocholic acid, threonine, tryptophan, tyrosine, and valine.

The cellular proto-oncogene c-Jun encodes a protein that, in combination with c-Fos, forms the AP-1 early response transcription factor. This proto-oncogene plays a key role in transcription and interacts with a large number of proteins affecting transcription and gene expression. It is also involved in proliferation and apoptosis of cells that form part of a number of tissues, including cells of the endometrium and glandular epithelial cells. G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, 03-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors.

When the improvement is made by consideration of patient or disease genotype, the consideration of patient or disease genotype can be, but is not limited to:
(a) diagnostic tools, techniques, kits and assays to confirm a patient's particular genotype;
(b) gene/protein expression chips and analysis;
(c) single nucleotide polymorphism (SNP) assessment;
(d) SNPs for histone deacetylase, ornithine decarboxylase, S-adenosyl methionine, GPCR's, protein kinases, telomerase, or jun;
(e) identification and measurement of metabolism enzymes and metabolites;
(f) mutation in specific wild-type and mutated genes;
(g) epigenetics via methylation and acetylation;
(h) mutations in genes for UGT, MGMT, BRCA, IDH, He 2, or EGFR;
(i) determination of expression for wild-type or mutated genes;
(j) detection or analysis of circulating tumor DNA or RNA;
(k) use of genome-wide sequencing;
(l) determination of the presence of A or G at genotypic marker −3156 of the UGT1A1 gene or at any position in linkage equilibrium with this genotypic marker wherein A positively correlates with irinotecan toxicity and G correlates with the absence of irinotecan toxicity, such that homozygosity for A indicates increased toxicity;
(m) a genotypic marker associated with polymorphisms in the TATA box within the promoter region for the UGT1A1 gene such that the presence of 7 TA repeats in the TATA box reduces expression of UGT1A1 and predisposes to increased toxicity;
(n) occurrence of variant alleles of MRP1;
(o) existence of single nucleotide polymorphisms in a region encoding APCDD1L, R3HCC1, OR5112, MKKS, EDEM3, or ACOX1;
(p) a polymorphism that is (G/G) for rs1792689, (C/T) or (C/C) for rs2268753; (G/G) for rs17776182, (A/A) for rs7570532, or (A/G) or (G/G) for rs4946935 which is favorable for efficacy of irinotecan when administered together with bevacizumab;
(q) a polymorphism that is (G/G) for rs1792689, (C/T) or (C/C) for rs2268753, (G/G) for rs17776182, (A/A) for rs7570532, and (A/G) or (G/G) for rs4946935, which is unfavorable for efficacy of irinotecan when administered together with bevacizumab; and
(r) the occurrence of a polymorphism rs1980576 in APCDD1L which is A in the wild-type and G in the mutant and where irinotecan has the strongest therapeutic effect when the genome is homozygous for A.

The use of gene chips is described in A. J. Lee & S. Ramaswamy, “DNA Microarrays in Biological Discovery and Patient Care” in Essentials of Genomic and Personalized Medicine (G. S. Ginsburg & H. F. Willard, eds., Academic Press, Amsterdam, 2010), ch. 7, pp. 73-88.

When the method is the use of single nucleotide polymorphism (SNP) analysis, the SNP analysis can be carried out on a gene selected from the group consisting of histone deacetylase, ornithine decarboxylase, VEGF, a prostate specific gene, c-Jun, and a protein kinase; SNP analysis can also be carried out on other genes and promoter sequences. The use of SNP analysis is described in S. Levy and Y.-H. Rogers, “DNA Sequencing for the Detection of Human Genome Variation” in Essentials of Genomic and Personalized Medicine (G. S. Ginsburg & H. F. Willard, eds., Academic Press, Amsterdam, 2010), ch. 3, pp. 27-37.

Still other genomic techniques such as copy number variation analysis and analysis of DNA methylation can be employed. Copy number variation analysis is described in C. Lee et al., “Copy Number Variation and Human Health” in Essentials of Genomic and Personalized Medicine (G. S. Ginsburg & H. F. Willard, eds., Academic Press, Amsterdam, 2010), ch. 5, pp. 46-59. DNA methylation analysis is described in S. Cottrell et al., “DNA Methylation Analysis: Providing New Insight into Human Disease” in Essentials of Genomic and Personalized Medicine (G. S. Ginsburg & H. F. Willard, eds., Academic Press, Amsterdam, 2010), ch. 6, pp. 60-72.

When the improvement is made by pre/post-treatment preparation, the pre/post-treatment preparation can be, but is not limited to, a specialized preparation of a patient prior to or after the use of a therapeutic agent selected from the group consisting of:
(a) use of colchicine or analogs;
(b) use of diuretics such as probenecid;
(c) use of uricase;
(d) non-oral use of nicotinamide;
(e) use of sustained release forms of nicotinamide;
(f) use of inhibitors of poly-ADP ribose polymerase;
(g) use of caffeine;
(h) use of leucovorin rescue;
(i) use of infection control;
(j) use of antihypertensives;
(k) use of alteration of stem cell populations;
(l) pretreatment to limit or prevent graft versus host (GVH) cytokine storm reactions;
(m) use of anti-inflammatories;
(n) anaphylactic reaction suppression; and
(o) use of anti-diarrhea treatments.

Uricosurics include, but are not limited to, probenecid, benzbromarone, and sulfinpyrazone. A particularly preferred uricosuric is probenecid. Uricosurics, including probenecid, may also have diuretic activity. Other diuretics are well known in the art, and include, but are not limited to, hydrochlorothiazide, carbonic anhydrase inhibitors, furosemide, ethacrynic acid, amiloride, and spironolactone.

Poly-ADP ribose polymerase inhibitors are described in G. J. Southan & C. Szabó, “Poly(ADP-Ribose) Inhibitors,” Curr. Med. Chem. 10: 321-240 (2003), and include nicotinamide, 3-aminobenzamide, substituted 3,4-dihydroisoquinolin-1(2H)-ones and isoquinolin-1(2H)-ones, benzimidazoles, indoles, phthalazin-1(2H)-ones, quinazolinones, isoindolinones, phenanthridinones, and other compounds.

Leucovorin rescue comprises administration of folinic acid (leucovorin) to patients in which methotrexate has been administered. Leucovorin is a reduced form of folic acid that bypasses dihydrofolate reductase and restores hematopoietic function. Leucovorin can be administered either intravenously or orally.

In one alternative, wherein the pre/post treatment is the use of a uricosuric, the uricosuric is probenecid or an analog thereof.

When the improvement is made by toxicity management, the toxicity management can be, but is not limited to, a method of toxicity management selected from the group consisting of:
(a) use of colchicine or analogs;
(b) use of diuretics such as probenecid;
(c) use of uricase;
(d) non-oral use of nicotinamide;
(e) use of sustained-release forms of nicotinamide;
(f) use of inhibitors of poly-ADP ribose polymerase;
(g) use of caffeine;
(h) leucovorin rescue;
(i) use of sustained-release allopurinol;
(j) non-oral use of allopurinol;
(k) use of bone marrow transplant stimulants, blood, platelet infusions, Neupogen, G-CSF, or GM-CSF;
(l) use of agents for pain management;
(m) use of anti-inflammatories;
(n) administration of fluids;
(o) administration of corticosteroids;
(p) administration of insulin control medications;
(q) administration of antipyretics;
(r) administration of anti-nausea treatments;
(s) administration of an anti-diarrhea treatment;
(t) administration of N-acetylcysteine;
(u) administration of antihistamines;
(v) administration of agents to limit or prevent mucositis;
(u) administration of agents to limit or prevent GVH reactions or cytokine storm reactions;
(v) administration of antifungal agents;
(w) administration of sodium thiosulfate;
(x) administration of glutathione;
(y) use of platelet transfusions;
(z) administration of epinephrine or anti-inflammatory corticosteroids for allergic or anaphylactic reactions;
(aa) administration of lidocaine or other local anesthetics;
(ab) administration of vasoconstrictors;
(ac) administration of vasodilators; and
(ad) administration of cephalosporin antibiotics.

Filgrastim is a granulocytic colony-stimulating factor (G-CSF) analog produced by recombinant DNA technology that is used to stimulate the proliferation and differentiation of granulocytes and is used to treat neutropenia; G-CSF can be used in a similar manner. GM-CSF is granulocyte macrophage colony-stimulating factor and stimulates stem cells to produce granulocytes (eosinophils, neutrophils, and basophils) and monocytes; its administration is useful to prevent or treat infection.

Anti-inflammatory agents are well known in the art and include corticosteroids and non-steroidal anti-inflammatory agents (NSAIDs). Corticosteroids with anti-inflammatory activity include, but are not limited to, hydrocortisone, cortisone, beclomethasone dipropionate, betamethasone, dexamethasone, prednisone, methylprednisolone, triamcinolone, fluocinolone acetonide, and fludrocortisone. Non-steroidal anti-inflammatory agents include, but are not limited to, acetylsalicylic acid (aspirin), sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naproxen, flurbiprofen, ketoprofen, fenoprofin, oxaprozin, mefenamic acid, meclofenamic acid, piroxicam, meloxicam, nabumetone, rofecoxib, celecoxib, etodolac, nimesulide, aceclofenac, alclofenac, alminoprofen, amfenac, ampiroxicam, apazone, araprofen, azapropazone, bendazac, benoxaprofen, benzydamine, bermoprofen, benzpiperylon, bromfenac, bucloxic acid, bumadizone, butibufen, carprofen, cimicoxib, cinmetacin, cinnoxicam, clidanac, clofezone, clonixin, clopirac, darbufelone, deracoxib, droxicam, eltenac, enfenamic acid, epirizole, esflurbiprofen, ethenzamide, etofenamate, etoricoxib, felbinac, fenbufen, fenclofenac, fenclozic acid, fenclozine, fendosal, fentiazac, feprazone, filenadol, flobufen, florifenine, flosulide, flubichin methanesulfonate, flufenamic acid, flufenisal, flunixin, flunoxaprofen, fluprofen, fluproquazone, furofenac, ibufenac, imrecoxib, indoprofen, isofezolac, isoxepac, isoxicam, licofelone, lobuprofen, lomoxicam, lonazolac, loxaprofen, lumaricoxib, mabuprofen, miroprofen, mofebutazone, mofezolac, morazone, nepafanac, niflumic acid, nitrofenac, nitroflurbiprofen, nitronaproxen, orpanoxin, oxaceprol, oxindanac, oxpinac, oxyphenbutazone, pamicogrel, parcetasal, parecoxib, parsalmide, pelubiprofen, pemedolac, phenylbutazone, pirazolac, pirprofen, pranoprofen, salicin, salicylamide, salicylsalicylic acid, satigrel, sudoxicam, suprofen, talmetacin, talniflumate tazofelone, tebufelone, tenidap, tenoxicam, tepoxalin, tiaprofenic acid, tiaramide, tilmacoxib, tinoridine, tiopinac, tioxaprofen, tolfenamic acid, triflusal, tropesin, ursolic acid, valdecoxib, ximoprofen, zaltoprofen, zidometacin, and zomepirac, and the salts, solvates, analogues, congeners, bioisosteres, hydrolysis products, metabolites, precursors, and prodrugs thereof.

The clinical use of corticosteroids is described in B. P. Schimmer & K. L. Parker, “Adrenocorticotropic Hormone; Adrenocortical Steroids and Their Synthetic Analogs; Inhibitors of the Synthesis and Actions of Adrenocortical Hormones” in Goodman & Gilman's The Pharmacological Basis of Therapeutics (L. L. Brunton, ed., 11^{th} ed., McGraw-Hill, New York, 2006), ch. 59, pp. 1587-1612.

Anti-nausea treatments include, but are not limited to, ondansetron, metoclopramide, promethazine, cyclizine, hyoscine, dronabinol, dimenhydrinate, diphenhydramine, hydroxyzine, medizine, dolasetron, granisetron, palonosetron, ramosetron, domperidone, haloperidol, chlorpromazine, fluphenazine, perphenazine, prochlorperazine, betamethasone, dexamethasone, lorazepam, and thiethylperazine.

Anti-diarrheal treatments include, but are not limited to, diphenoxylate, difenoxin, loperamide, codeine, racecadotril, octreoside, and berberine.

N-acetylcysteine is an antioxidant and mucolytic that also provides biologically accessible sulfur.

Antihistamines include, but are not limited to, acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimetindene, diphenhydramine, ebastine, embramine, fexofenadine, levocabastine, levocetirizine, loratadine, phenindamine, pheniramine, phenyltoloxamine, rupatadine, tripelennamine, and triprolidine.

Agents to limit or prevent mucositis include, but are not limited to, palifermin, episil, and dusquetide.

Agents to limit or prevent graft-versus-host (GVH) reactions or cytokine storm reactions include, but are not limited to, glucocorticoids such as prednisone, betamethasone, or dexamethasone, cyclosporine, tacrolimus, sirolimus, pentostatin, etanercept, alemtuzumab, and ibrutinib.

Antifungal agents include, but are not limited to, ketoconazole, itraconazole, fluconazole, fosfluconazole, voriconazole, posaconazole, isavuconazole, griseofulvin, amphotericin B, candidicin, filipin, hamycin, natamycin, nystatin, rimocidin, bifonazole, butoconazole, clotrimazole, econazole, fenticonazole, isoconazole, luliconazole, miconazole, omoconazole, oxiconazole, sertaconazole, sulconazole, tioconazole, albaconazole, efinaconazole, epoxiconazole, propiconazole, terconazole, abafungin, butenafine, naftifine, terbinafine, anidulafungin, caspofungin, micafungin, ibrexafungerp, acrisorcin, amorolfine, ciclopirox, clioquinol, chlorophetanol, iodoquinol, 5-fluorocytosine, fumagillin, miltefosine, nikkomycin, orotomide, piroctone olamine, pentanenitrile, tolnaftate, and undecylenic acid.

Local anesthetics include, but are not limited to, lidocaine, benzocaine, chloroprocaine, cyclomethycaine, dimethocaine, piperocaine, propoxycaine, procaine, proparacaine, tetracaine, articaine, bupivacaine, cinchocaine, etidocaine, levobupivacaine, mepivacaine, prilocaine, ropivacaine, and trimecaine.

Vasoconstrictors include, but are not limited to, epinephrine, caffeine, ergometrine, naphazoline, oxymetazoline, phenylephrine, propylhexidine, and pseudoephedrine.

Vasodilators include, but are not limited to, methyldopa, clonidine hydrochloride, guanabenz acetate, guanfacine hydrochloride, hydralazine, and minoxidil, as well as angiotensin II receptor blockers, angiotensin converting enzyme inhibitors, and calcium channel blockers.

Cephalosporin antibiotics include, but are not limited to, cefalexin, cefadroxil, cefazolin, cefapirin, cefacetrile, cefaloglycin, cefalonium, cefaloridine, cefatrizine, cefazaflur, cefazedone, cefadrine, cefroxadine, ceftezole, cefuroxime, cefprozil, cefactor, cefonicid, cefuzonam, cefoxitin, cefotetan, cefmetazole, cefminox, cefbuperazone, cefotiam, cefdinir, ceftriaxone, ceftazidime, cefixime, cefpodoxime, ceftiofur, cefotaxime, ceftizoxime, cefditoren, ceftibuten, cefovecin, cefdaloxime, cefcapene, cefetamet, cefmenoxime, cefodizime, cefpimizole, cefteram, ceftiolene, cefoperazone, cefepime, cefiderocol, cefquinome, cefclidine, cefluprenam, cefoselis, cefpirome, ceftaroline, ceftolozane, ceftobiprole, cefaloram, cefaparole, cefcanel, cefedrolor, cefempidone, cefetrizole, cefivitril, cefmatilen, cefmepidium, cefoxazole, cefrotil, cefsumide, cefuracetime, and nitrocefin.

When the improvement is made by pharmacokinetic/pharmacodynamic monitoring, the pharmacokinetic/pharmacodynamic monitoring can be, but is not limited to, a method of pharmacokinetic/pharmacodynamic monitoring selected from the group consisting of:
(a) multiple determinations of drug plasma levels;
(b) multiple determinations of metabolites in the blood or urine;
(c) measurement of polyamines;
(d) determination of density of LAT-1 surface receptors;
(e) use of gene sequencing to determine levels of activation of specific genes;
(f) determination of levels of immune effectors;
(g) determination of level of prodrug conversion of irinotecan to SN-38;
(h) determination of level of glucuronidation of SN-38.

Typically, determination of drug plasma levels or determination of at least one metabolite in blood or urine is carried out by immunoassays. Methods for performing immunoassays are well known in the art, and include radioimmunoassay, ELISA (enzyme-linked immunosorbent assay), competitive immunoassay, immunoassay employing lateral flow test strips, and other assay methods.

When the improvement is made by use of a drug combination, the drug combination can be, but is not limited to, a drug combination selected from the group consisting of:
(a) use with other topoisomerase inhibitors;
(b) use with fraudulent nucleosides;
(c) use with fraudulent nucleotides;
(d) use with thymidylate synthetase inhibitors;
(e) use with signal transduction inhibitors;
(f) use with cisplatin or platinum-containing analogs;
(g) use with alkylating agents such as BCNU, Gliadel wafers, CCNU, bendamustine (Treanda), or temozolomide (Temodar);
(h) use with anti-tubulin agents;
(i) use with antimetabolites;
(j) use with berberine;
(k) use with apigenin;
(l) use with amonafide;
(m) use with colchicine or colchicine analogs;
(n) use with genistein;
(o) use with cytarabine;
(p) use with vinca alkaloids;
(q) use with 5-fluorouracil;
(r) use with curcumin;
(s) use with NF-κB inhibitors;
(t) use with rosmarinic acid;
(u) use with dianhydrogalactitol;
(v) use with dibromodulcitol;
(w) use with biological therapies such as antibodies such as Avastin, Rituxan, Herceptin, Erbitux, PD-1 and PDL-1 inhibitors;
(x) use with prednimustine;
(y) use with DNA and RNA therapeutics;
(z) use with Braf inhibitors;
(aa) use with BTK inhibitors;
(ab) use with 5-azacytidine;
(ac) use with decitabine;
(ad) use with PARP inhibitors;
(ae) use with hypomethylating agents;
(af) use with histone deacetylase inhibitors;
(ag) use with thalidomide;
(ah) use with trifluridine;
(ai) use with tipiracil hydrochloride;
(aj) use with aflibercept;
(ak) use with 5-(5-(2-(3-aminopropoxy)-6-methoxyphenyl)-1H-pyrazol-3-ylamino)pyrazine-2-carbonitrile;
(al) use with EGFR inhibitors;
(am) use with VEGF inhibitors;
(an) use with a humanized anti-EGFR IgG1 antibody;
(ao) use with 4-iodo-3-nitrobenzamide or metabolites thereof;
(ap) use with immunotherapies including: antibodies binding to alpha-PDL1, alpha-44BB, alpha-CTLA4, or alpha-OX40; or atezolizumab, avelimumab, nivolumab, pembrolizumab, ipilimumab, tremelimumab, or durvalumab; Chk1-directed therapeutic agents such as prexasertib; topoisomerase 2-directed therapeutic agents such as aldozurubicin; DNA inhibitors such as lurbinectedin; and Notch ADC-modulating agents such as rovalpituzumab tesirine; use with dilpacimab; and
(aq) use with an MRP inhibitor such as valspodar (SDZ-PSC 833), tert-butyl 2-[(3S,6S,9S,15S,21S,24S,27S,30S)-15,18-bis[(2S)-butan-2-yl]-6-[(4-methoxyphenyl)methyl]-3,10,16,19,22,28-hexamethyl-2,5,8,11,14,17,20,23,26,29-decaoxo-9,24,27-tri(propan-2-yl)-4-oxa-1,7,10,13,16,19,22,25,28-nonazabicyclo[28.4.0]tetratriacontan-21-yl]acetate (SDZ 280-446), sodium 3-[[3-[(E)-2-(7-chloroquinolin-2-yl)ethenyl]phenyl]-[3-(dimethylamino)-3-oxopropyl]sulfanylmethyl]sulfanylpropanoate (MK571), dofequidar (MS209), 2-(4-benzhydrylpiperazin-1-yl)ethyl 5-[(4R,6R)-4,6-dimethyl-2-oxo-1,3,2λ-5-dioxaphosphinan-2-yl]-2,6-dimethyl-4-(3-nitrophenyl)pyridine-3-carboxylate (PAK-104p), verapamil, benzbromarone, dipyridamole, furosemide, gamma-GS(naphthyl)cysteinyl-glycine diethyl ester, genistein, quinidine, rifampicin, mifepristone (RU-486), or sulfinpyrazone.

Topoisomerase inhibitors other than camptothecin-based topoisomerase inhibitors include, but are not limited to, lamellarin D, amsacrine, etoposide, etoposide phosphate, teniposide, doxorubicin, and 4-[2-(3,5-dioxo-1-piperazinyl)-1-methylpropyl]piperazine-2,6-dione (ICRF-193). Etoposide is an anticancer agent that acts primarily as a topoisomerase II inhibitor. Etoposide forms a ternary complex with DNA and the topoisomerase II enzyme, prevents re-ligation of the DNA strands and thus induces DNA strand breakage and promotes apoptosis of the cancer cells.

Fraudulent nucleosides include, but are not limited to, cytosine arabinoside, gemcitabine, and fludarabine; other fraudulent nucleosides are known in the art.

Fraudulent nucleotides include, but are not limited to, tenofovir disoproxil fumarate and adefovir dipivoxil; other fraudulent nucleotides are known in the art.

Thymidylate synthetase inhibitors include, but are not limited to, raltitrexed, pemetrexed, nolatrexed, ZD9331, GS7094L, fluorouracil, and BGC 945.

Signal transduction inhibitors are described in A. V. Lee et al., “New Mechanisms of Signal Transduction Inhibitor Action: Receptor Tyrosine Kinase Down-Regulation and Blockade of Signal Transactivation,” Clin. Cancer Res. 9: 516s (2003).

Platinum-containing analogs of cisplatin include carboplatin, dicycloplatin, lipoplatin, miriplatin, nedaplatin, oxaliplatin, picoplatin, and satraplatin.

Alkylating agents include, but are not limited to, Shionogi 254-S, aldo-phosphamide analogues, altretamine, anaxirone, Boehringer Mannheim BBR-2207, bendamustine, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)₂, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, melphalan, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromustine, Tanabe Seiyaku TA-077, tauromustine, temozolomide, teroxirone, tetraplatin and trimelamol, as described in U.S. Pat. No. 7,446,122 by Chao et al. Alkylating agents can include nitroso-containing alkylating agents.

Anti-tubulin agents include, but are not limited to, colchicine and analogs of colchicine. Colchicine is a tricyclic alkaloid that exerts its activity by binding to the protein tubulin. Analogs of colchicine include, but are not limited to, colchiceinamide, N-desacetylthiocolchicine, demecolcine, N-acetyliodocolchinol, trimethylcolchicinic acid (TMCA) methyl ether, N-acetylcolchinol, TMCA ethyl ether, isocolchicine, isocolchiceinamide, iso-TMCA methyl ether, colchiceine, TMCA, N-benzoyl TMCA, colchicosamide, colchicoside, colchinol and colchinoic acid (M. H. Zweig & C. F. Chignell, “Interaction of Some Colchicine Analogs, Vinblastine and Podophyllotoxin with Rat Brain Microtubule Protein,” Biochem. Pharmacol. 22: 2141-2150 (1973) and B. Yang et al., “Syntheses and Biological Evaluation of Ring C-Modified Colchicine Analogs,” Bioorg. Med. Chem. Lett. 20: 3831-3833 (2010)).

Antimetabolites include, but are not limited to, base analogs such as purine analogs or pyrimidine analogs, nucleoside analogs, nucleotide analogs, and antifolates.

Berberine has antibiotic activity and prevents and suppresses the expression of pro-inflammatory cytokines and E-selectin, as well as increasing adiponectin expression.

Apigenin is a flavone that can reverse the adverse effects of cyclosporine and has chemoprotective activity, either alone or derivatized with a sugar.

Genistein is an isoflavone with the systemic name 5,7-dihydroxy-3-(4-hydroxyphenyl)chromen-4-one. Genistein has a number of biological activities, including activation of PPARs, inhibition of several tyrosine kinases, inhibition of topoisomerase, antioxidative activity, activation of Nrf2 antioxidative response, activation of estrogen receptor beta, and inhibition of the mammalian hexose transporter GLUT2.

Cytarabine is a nucleoside analog replacing the ribose with arabinose. It can be incorporated into DNA and also inhibits both DNA and RNA polymerases and nucleotide reductase. It is particularly useful in the treatment of acute myeloid leukemia and acute lymphocytic leukemia, but can be used for other malignancies and in various drug combinations.

Vinca alkaloids include vinblastine, vincristine, vindesine, and vinorelbine.

The compound 5-fluorouracil is a base analog that acts as a thymidylate synthase inhibitor and thereby inhibits DNA synthesis. When deprived of a sufficient supply of thymidine, rapidly dividing cancer cells die by a process known as thymineless death.

Curcumin is believed to have anti-neoplastic, anti-inflammatory, antioxidant, anti-ischemic, anti-arthritic, and anti-amyloid properties and also has hepatoprotective activity.

NF-κB is a protein complex that controls transcription of DNA, cytokine production, and cell survival. NF-κB is involved in cellular responses to stimuli such as stress, cytokines, free radicals, heavy metals, ultraviolet radiation, oxidized low-density lipoprotein, and antigens of bacterial or viral antigens. NF-κB inhibitors include, but are not limited, to, bortezomib, denosumab, disulfiram, olmesartan, dithiocarbamates, (−)-DHMEQ, PBS-1086, IT-603, IT-901, BAY-11-7082, palmitoylethanolamide, and iguratimod.

Rosmarinic acid is a naturally-occurring phenolic antioxidant that also has anti-inflammatory activity.

Dianhydrogalactitol and dibromodulcitol are epoxy-containing sugar derivatives that are alkylating agents that alkylate DNA and act as anti-neoplastic agents. Dibromodulcitol can act as a prodrug of dianhydrogalactitol.

Avastin (bevacizumab) is a recombinant humanized monoclonal antibody that blocks angiogenesis by inhibiting vascular endothelial growth factor A (VEGF) and that is used to treat a number of malignancies, including colorectal cancer, lung cancer, breast cancer, renal cancers, ovarian cancer, and cervical cancer, as well as a number of non-malignant conditions such as age-related macular degeneration and diabetic retinopathy. Rituxan (rituximab) is a chimeric monoclonal antibody that binds to the B cell surface antigen CD20 and that is used to treat non-Hodgkin's lymphoma, chronic lymphocytic leukemia, and a number of non-malignant conditions including rheumatoid arthritis, vasculitis, and pemphigus vulgaris. Herceptin (trastuzumab) is a monoclonal antibody targeting HER2 that induces an immune-mediated response that causes internalization and recycling of HER2 and may upregulate cell cycle inhibitors; it is used to treat breast cancer. Erbitux (cetuximab) is a chimeric monoclonal antibody that inhibits epidermal growth factor receptor (EGFR) and is used to treat squamous cell carcinoma of the head and neck.

PD-1 inhibitors include pembrolizumab, nivolumab, cemiplimab, JTX-4014, spartalizumab, camrelizumab, sintilimab, tislelizumab, toripalimab, dostarlimab, MGA012, AMP-224, and AMP-514. PD-L1 inhibitors include atezolizumab, avelumab, durvalumab, KN035, AUNP12, CA-170, and BMS-986189. PL-1 and PDL-1 inhibitors are checkpoint inhibitors and can be used to treat malignancies by preventing the malignancy from evading the immune system.

Avastin (bevacizumab) is a recombinant humanized monoclonal antibody that blocks angiogenesis by inhibiting vascular endothelial growth factor A (VEGF) and that is used to treat a number of malignancies, including colorectal cancer, lung cancer, breast cancer, renal cancers, ovarian cancer, and cervical cancer, as well as a number of non-malignant conditions such as age-related macular degeneration and diabetic retinopathy. Rituxan (rituximab) is a chimeric monoclonal antibody that binds to the B cell surface antigen CD20 and that is used to treat non-Hodgkin's lymphoma, chronic lymphocytic leukemia, and a number of non-malignant conditions including rheumatoid arthritis, vasculitis, and pemphigus vulgaris. Herceptin (trastuzumab) is a monoclonal antibody targeting HER2 that induces an immune-mediated response that causes internalization and recycling of HER2 and may upregulate cell cycle inhibitors; it is used to treat breast cancer. Erbitux (cetuximab) is a chimeric monoclonal antibody that inhibits epidermal growth factor receptor (EGFR) and is used to treat squamous cell carcinoma of the head and neck.

Prednimustine is an alkylating agent that is an ester formed from prednisolone and chlorambucil and is used in the treatment of leukemias and lymphomas.

Braf inhibitors include vemurafenib, GDC-0879, PLX-4720, sorafenib, dabrafenib, and LGX818 and are used to treat metastatic melanoma.

BTK inhibitors include ibrutinib, acalabrutinib, zanubrutinib, tirabrutinib, tolebrutinib, evobrutinib, ABBV-105, fenebrutinib, pirtobrutinib, GS-4059, spebrutinib, and HM71224.

5-azacytidine and decitabine are antimetabolites that are analogs of cytidine or 2′-deoxycytidine and are used in the treatment of myelodysplastic syndrome.

Agents inducing hypomethylation include 5-azacytidine and decitabine, as well as pseudoisocytidine and 5-fluoro-2′-deoxycytidine. Histone deacetylase inhibitors include vorinostat and romidepsin. The use of histone deacetylase inhibitors is also described in United States Patent Application Publication No. 2011/0105474 by Thaler et al. These histone deacetylase inhibitors include, but are not limited to, (E)-N-hydroxy-3-{4-[(E)-3-(4-methyl-piperazin-1-yl)-3-oxo-propenyl]-phenyl}-acrylamide; (E)-N-hydroxy-3-{3-[(E)-3-(4-methyl-piperazin-1-yl)-3-oxo-propenyl]-phenyl}-acrylamide; (E)-N-hydroxy-3-{3-[(E)-3-oxo-3-(4-phenyl-piperazin-1-yl)-propenyl]-phenyl}-acrylamide; and (E)-3-[3-((E)-3-[1,4′]bipiperidinyl-1′-yl-3-oxo-propenyl)-phenyl]-N-hydroxy-acrylamide. Additional histone deacetylase inhibitors, including spirocyclic derivatives, are described in United States Patent Application Publication No. 2011/039840 by Varasi et al. Prodrugs of histone deacetylase inhibitors are described in U.S. Pat. No. 8,227,636 to Miller et al. Histone deacetylase inhibitors are described in U.S. Pat. No. 8,222,451 to Kozikowski et al. Histone deacetylase inhibitors, including disubstituted aniline compounds, are also described in U.S. Pat. No. 8,119,685 to Heidebrecht et al. Histone deacetylase inhibitors, including aryl-fused spirocyclic compounds, are also described in U.S. Pat. No. 8,119,852 to Hamblett et al.

Leucovorin, also known as folinic acid, is a 5-formyl derivative of tetrahydrofolic acid and functions as an equivalent to folic acid by conversion to reduced folic acid derivatives; its conversion is not dependent on the catalytic activity of dihydrofolate reductase and thus is not prevented by administration of dihydrofolate reductase inhibitors such as methotrexate.

Trifluridine is a nucleoside analog that has antiviral and anti-neoplastic activity.

Tipiracil hydrochloride is a thymidine phosphorylase inhibitor that is typically used as an anti-neoplastic agent in combination with trifluridine.

Aflibercept is a recombinant fusion protein consisting of vascular endothelial growth factor (VEGF)-binding portions from the extracellular domains of human VEGF receptors 1 and 2, that are fused to the Fc portion of the human IgG1 immunoglobulin and is a VEGF inhibitor; it has anti-neoplastic activity.

EGFR inhibitors include, but are not limited to, gefitinib, erlotinib, afatinib, brigatinib, icotinib, cetuximab, osimertinib, panitumumab, zalutumumab, nimotuzumab, matuzumab, and lapatinib. These inhibitors include both monoclonal antibodies or their derivatives and small molecules.

VEGF inhibitors include, but are not limited to, bevacizumab, ranibizumab, sunitinib, axitinib, pazopanib, and pegaptanib. These inhibitors include both monoclonal antibodies or their derivatives and small molecules.

Inhibitors of the enzyme poly-ADP ribose polymerase (PARP) have been developed for multiple indications, especially for treatment of malignancies. Several forms of cancer are more dependent on the activity of PARP than are non-malignant cells.

The enzyme PARP catalyzes the polymerization of poly-ADP ribose chains, typically attached to a single-strand break in cellular DNA. The coenzyme NAD⁺ is required as a substrate for generating ADP-ribose monomers to be polymerized, nicotinamide is the leaving group during polymerization, in contrast to pyrophosphate which is the leaving group during normal DNA or RNA synthesis, which leaves a pyrophosphate as the linking group between adjacent ribose sugars in the chain rather than phosphate as occurs in normal DNA or RNA. The PARP enzyme comprises four domains: a DNA-binding domain, a caspase-cleaved domain, an auto-modification domain, and a catalytic domain. The DNA-binding domain comprises two zinc finger motifs. In the presence of damaged DNA, the DNA-binding domain will bind the DNA and induce a conformational shift. PARP can be inactivated by caspase-3 cleavage, which is a step that occurs in programmed cell death (apoptosis).

Several PARP enzymes are known, including PARP1 and PARP2. Of these two enzymes, PARP1 is responsible for most cellular PARP activity. The binding of PARP1 to single-strand breaks in DNA through the amino-terminal zinc finger motifs recruits XRCC1, DNA ligase III, DNA polymerase p, and a kinase to the nick. This is known as base excision repair (BER). PARP2 has been shown to oligomerize with PARP1, and the oligomerization stimulates catalytic activity. PARP2 is also therefore implicated in BER.

PARP1 inhibitors inhibit the activity of PARP1 and thus inhibit the repair of single-strand breaks in DNA. When such breaks are unrepaired, subsequent DNA replication can induce double-strand breaks. The proteins BRCA1, BRCA2, and PALB2 can repair double-strand breaks in DNA by the error-free homologous recombinational repair (HRR) pathway. In tumors with mutations in the genes BRCA1, BRCA2, or PALB1, these double-strand breaks cannot be efficiently repaired, leading to cell death. Normal cells do not replicate their DNA as frequently as tumor cells, and normal cells that lack mutated BRCA1 or BRCA2 proteins can still repair these double-strand breaks through homologous repair. Therefore, normal cells are less sensitive to the activity of PARP inhibitors than tumor cells.

Some tumor cells that lack the tumor suppressor PTEN may be sensitive to PARP inhibitors because of downregulation of Rad51, a critical homologous recombination component. Tumor cells that are low in oxygen are also sensitive to PARP inhibitors.

PARP inhibitors are also considered potential treatments for other life-threatening diseases, including stroke and myocardial infarction, as well as for long-term neurodegenerative diseases (G. Graziani & C. Szabó, “Clinical Perspectives of PARP Inhibitors,” Pharmacol. Res. 52: 109-118 (2005)).

A number of PARP inhibitors are known in the art. PARP inhibitors include, but are not limited to, iniparib, talazoparib, olaparib, rucaparib, veliparib, CEP-9722 (a prodrug of CEP-8983 (11-methoxy-4,5,6,7-tetrahydro-1H-cyclopenta[a]pyrrolo[3,4-c]carbazole-1,3(2H)-dione), MK 4827 ((S)-2-(4-(piperidin-3-yl)phenyl)-2H-indazole-7-carboxamide), and BGB-290. Other PARP inhibitors are described below.

U.S. Pat. No. 9,073,893 to Papeo et al. discloses 3-oxo-2,3-dihydro-1H-indazole-4-carboxamide derivatives as PARP inhibitors.

U.S. Pat. No. 9,062,061 by Honda et al. discloses a PARP inhibitor of Formula (PA-I):

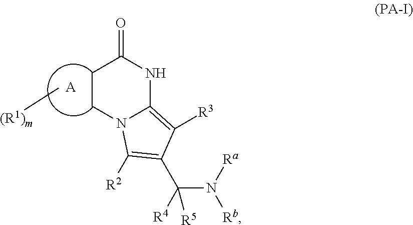

wherein:
(1) R¹ represents a halogen atom, a lower alkyl group, a hydroxy group, a lower alkoxy group, an amino group, a nitro group or a cyano group;
(2) R² and R³ may be the same or different and each represent a hydrogen atom, a halogen atom or a lower alkyl group;
(3) R⁴ and R⁵ may be the same or different and each represent a hydrogen atom, a deuterium atom or a lower alkyl group, or R⁴ and R⁵ may form an oxo group; R^{a} and R^{b} may be the same or different and each represent a hydrogen atom, a lower alkyl group optionally having a substituent or an aryl group optionally having a substituent; R^{a} and R^{b} may bind to each other to form a nitrogen-containing heterocyclic ring which may be substituted by one or plural R^{c};
(4) R^{c} represents a lower alkyl group optionally having a substituent, a lower cycloalkyl group optionally having a substituent, an aryl group optionally having a substituent, a heterocyclic group optionally having a substituent, a hydroxy group, a lower alkoxy group optionally having a substituent, a lower alkylcarbonyl group optionally having a substituent, a lower cycloalkylcarbonyl group optionally having a substituent, a lower alkylaminocarbonyl group optionally having a substituent, a lower cycloalkylaminocarbonyl group optionally having a substituent, a lower alkoxycarbonyl group optionally having a substituent, an amino group, a lower alkylamino group or a carboxyl group;
(5) ring A represents a benzene ring or an unsaturated heteromonocyclic ring; and
(6) m represents 0, 1 or 2.

U.S. Pat. No. 9,062,043 to Chua et al. discloses fused tricyclic PARP inhibitors, including a compound of Formula (PA-II):

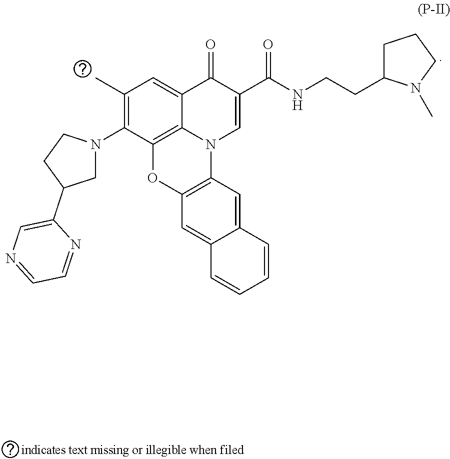 [SMILES: Cc1cc2c(=O)c(C(=O)NCCC3CCCN3C)cn3c2c(c1N1CCC(c2cnccn2)C1)Oc1cc2ccccc2cc1-3]

U.S. Pat. No. 9,018,201 to Chu et al discloses dihydropyridophthalazinone inhibitors of PARP.

U.S. Pat. No. 8,993,594 to Papeo et al. discloses substituted isoquinolin-1(2H)-one derivatives as inhibitors of PARP.

U.S. Pat. No. 8,980,902 to Brown et al. discloses substituted benzamide PARP inhibitors.

U.S. Pat. No. 8,946,221 to Mevellec et al. discloses phthalazine derivatives as PARP inhibitors.

U.S. Pat. No. 8,889,866 to Angibaud et al. discloses tetrahydrophenanthridinones and tetrahydrocyclopentaquinolinones as PARP inhibitors.

U.S. Pat. No. 8,883,787 to Xu et al. discloses diazabenzo[de]anthracen-3-one derivatives as PARP inhibitors.

U.S. Pat. No. 8,877,944 to Papeo et al. discloses substituted 3-oxo-2,3-dihydro-1H-isoindole-4-carboxamide derivatives as PARP inhibitors.

U.S. Pat. No. 8,778,966 to Vialard et al. discloses substituted quinolinone derivatives as PARP inhibitors.

U.S. Pat. No. 8,697,736 to Penning et al. discloses 1H-benzimidazole-4-carboxamides as PARP inhibitors.

U.S. Pat. No. 8,669,249 to Brown et al. discloses PARP inhibitors including: 2-methyl-6-((4-phenylpiperidin-1-yl)methyl)-2H-benzo[b][1,4]oxazin-3(4H)-one; 2-methyl-6-((4-phenylpiperazin-1-yl)methyl)-2H-benzo[b][1,4]oxazin-3(4H)-one; and 6-((4-(4-fluorophenyl)-5,6-dihydropyridin-1(2H)-yl)methyl)-2-methyl-2H-benzo[b][1,4]oxazin-3(4H)-one, as well as additional compounds.

U.S. Pat. No. 8,663,884 to Kennis et al. discloses quinazolinedione derivatives as PARP inhibitors.

U.S. Pat. No. 8,623,872 to Guillemont et al. discloses quinazolinone derivatives as PARP inhibitors.

U.S. Pat. No. 8,546,368 to Penning et al. discloses pyrazoquinolones as PARP inhibitors, including 7,9-dimethyl-1,2,3,4,6,7-hexahydro-5H-pyrazolo[3,4-h]-1,6-naphthyridin-5-one.

U.S. Pat. No. 8,541,417 to Brown et al. discloses PARP inhibitors, including: 3-(hydroxymethyl)pyrido[2,3-e]pyrrolo[1,2-c]pyrimidin-6(5H)-one; N-ethyl-4-(4-((6-oxo-5,6-dihydropyrido[2,3-e]pyrrolo[1,2-c]pyrimidin-3-yl)methyl)piperazin-1-yl)benzamide; and N-methyl-4-(4-((6-oxo-5,6-dihydropyrido[2,3-e]pyrrolo[1,2-c]pyrimidin-3-yl)methyl)piperazin-1-yl)benzamide, as well as additional compounds.

U.S. Pat. No. 8,541,403 to Chu et al. discloses dihydropyridophthalazinone derivatives as PARP inhibitors.

U.S. Pat. No. 8,513,433 to Panicker et al. discloses inhibitors of PARP, including benzyl 2-(4-carbamoyl-1H-benzo[d]imidazol-2-yl)indoline-1-carboxylate; 2-(indolin-2-yl)-1H-benzo[d]imidazole-4-carboxamide; tert-butyl 2-(4-carbamoyl-1H-benzo[d]imidazol-2-yl)-3,4-dihydroquinoline-1(2H)-carboxylate; and 2-(1,2,3,4-tetrahydroquinolin-2-yl)-1H-benzo[d]imidazole-4-carboxamide, as well as additional compounds.

U.S. Pat. No. 8,470,825 to Xu et al. discloses substituted diazabenzo[de]anthracen-3-one compounds as PARP inhibitors.

U.S. Pat. No. 8,420,650 to Wang et al. discloses dihydropyridophthalazinone inhibitors of PARP.

U.S. Pat. No. 8,362,030 to Ingenito et al. discloses tricyclic PARP inhibitors, including: N-methyl[4-(6-oxo-3,4,5,6-tetrahydro-2H-azepino[5,4,3-cd]indazol-2-yl)phenyl]methanaminium trifluoroacetate; N,N-dimethyl[4-(6-oxo-3,4,5,6-tetrahydro-2H-azepino[5,4,3-cd]indazol-2-yl)phenyl]methanaminium trifluoroacetate; and N²,N²-dimethyl-N-[4-(1-oxo-1,2,3,4-tetrahydroazepino[3,4,5-hi]indolizin-5-yl)phenyl]glycinamide, as well as additional compounds.

U.S. Pat. No. 8,354,413 to Jones et al. discloses quinolin-4-one and 4-oxodihydrocinnoline derivatives as PARP inhibitors, including: 1-[3-(8-aza-1-azoniaspiro[4.5]dec-8-ylcarbonyl)-4-fluorobenzyl]-4-oxo-1,4-dihydroquinolinium bis(trifluoroacetate); 1-[4-fluoro-3-({4-[2-(4-fluorobenzyl)prolyl]piperazin-1-yl}carbonyl)benzyl]quinolin-4(1H)-one; and 1-[3-(8-aza-1-azoniaspiro[4.5]dec-8-ylcarbonyl)-4-fluorobenzyl]-4-oxo-1,4-dihydrocinnolin-1-ium bis(trifluoroacetate), as well as additional compounds.

U.S. Pat. No. 8,268,827 to Branca et al. discloses pyridazinone derivatives as PARP inhibitors, including: 6-{4-fluoro-3-[(3-oxo-4-phenylpiperazin-1-yl)carbonyl]benzyl}-4,5-dimethyl-3-oxo-2,3-dihydropyridazin-1-ium trifluoroacetate; 6-{3-[(4-cyclohexyl-3-oxopiperazin-1-yl)carbonyl]-4-fluorobenzyl}-4,5-dimethyl-3-oxo-2,3-dihydropyridazin-1-ium trifluoroacetate; 6-{3-[(4-cyclopentyl-3-oxopiperazin-1-yl)carbonyl]-4-fluorobenzyl}-4,5-dimethylpyridazin-3(2H)-one; and 6-{4-fluoro-3-[(3-oxo-4-phenylpiperazin-1-yl)carbonyl]benzyl}-4,5-dimethylpyridazin-3(2H)-one hydrochloride, as well as additional compounds.

U.S. Pat. No. 8,217,070 to Zhu et al. discloses 2-substituted-1H-benzimidazole-4-carboxamides as PARP inhibitors, including: 2-(1-aminocyclopropyl)-1H-benzimidazole-4-carboxamide; 2-[1-(isopropylamino)cyclopropyl]-1H-benzimidazole-4-carboxamide; 2-[1-(cyclobutylamino)cyclopropyl]-1H-benzimidazole-4-carboxamide; and 2-{1-[(3,5-dimethylbenzyl)amino]cyclopropyl}-1H-benzimidazole-4-carboxamide, as well as additional compounds.

U.S. Pat. No. 8,188,103 to Van der Aa et al. discloses substituted 2-alkyl quinazolinone derivatives as PARP inhibitors.

U.S. Pat. No. 8,173,682 to Weintraub et al. discloses 2,3,5-substituted pyridone derivatives as PARP inhibitors, including: 5-(5-ethyl-2-methyl-6-oxo-1,6-dihydro-pyridin-3-yl)-thiophene-2-sulfonic acid [3-(3-hydroxy-pyrrolidin-1-yl)-propyl]-amide hydrochloride; and 5-(5-ethyl-2-methyl-6-oxo-1,6-dihydropyridin-3-yl)thiophene-2-sulfonic acid [2-(1-methylpyrrolidin-2-yl)ethyl]amide hydrochloride, as well as additional compounds.

U.S. Pat. No. 8,129,382 to Kalish et al. discloses PARP inhibitors of Formula (PA-III)

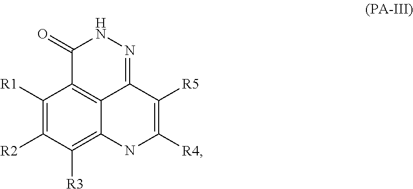

wherein:
(1) R1 is H, halogen, alkoxy, or lower alkyl;
(2) R2 is H, halogen, alkoxy, or lower alkyl;
(3) R3 is independently H, amino, hydroxy, —N—N, halogen-substituted amino, —O-alkyl, —O-aryl, or an optionally substituted alkyl, alkenyl, alkynyl, cycloalkyl, heterocycloalkyl, aryl, heteroaryl, —COR8, where R8 is H, —OH an optionally substituted alkyl, alkenyl, alkynyl, cycloalkyl, heterocycloalkyl, aryl or heteroaryl, or —OR6 or —NR6R7 where R6 and R7 are each independently hydrogen or an optionally substituted alkyl, alkenyl, alkynyl, cycloalkyl, heterocycloalkyl, aryl or heteroaryl;
(4) R4 is independently H, amino, hydroxy, —N—N, —CO—N—N, halogen-substituted amino, —O-alkyl, —O-aryl, or an optionally substituted alkyl, alkenyl, alkynyl, cycloalkyl, heterocycloalkyl, aryl, heteroaryl, —COR8, where R8 is H, —OH an optionally substituted alkyl, alkenyl, alkynyl, cycloalkyl, heterocycloalkyl, aryl or heteroaryl, or —OR6 or —NR6R7 where R6 and R7 are each independently hydrogen or an optionally substituted alkyl, alkenyl, alkynyl, cycloalkyl, heterocycloalkyl, aryl or heteroaryl; and
(5) R5 is independently H, amino, hydroxy, —N—N, —CO—N—N, halogen-substituted amino, —O-alkyl, —O-aryl, or an optionally substituted alkyl, alkenyl, alkynyl, cycloalkyl, heterocycloalkyl, aryl, heteroaryl, —COR8, where R8 is H, —OH an optionally substituted alkyl, alkenyl, alkynyl, cycloalkyl, heterocycloalkyl, aryl or heteroaryl, or —OR6 or —NR6R7 where R6 and R7 are each independently hydrogen or an optionally substituted alkyl, alkenyl, alkynyl, cycloalkyl, heterocycloalkyl, aryl or heteroaryl.

U.S. Pat. No. 8,088,760 to Chu et al. discloses benzoxazole carboxamide inhibitors of PARP, including: 2-(4-((methylamino)methyl)phenyl)benzo[d]oxazole-4-carboxamide; 2-(2-methylpyrrolidin-2-yl)benzo[d]oxazole-4-carboxamide; 2-(4-((methylamino)methyl)phenyl)benzo[d]oxazole-7-carboxamide; 2-(2-methylpyrrolidin-2-yl)benzo[d]oxazole-7-carboxamide; and 2-(pyrrolidin-2-yl)benzo[d]oxazole-4-carboxamide, as well as additional compounds.

U.S. Pat. No. 8,071,623 to Jones et al. discloses amide-substituted indazoles as PARP inhibitors, including: 2-(4-piperidin-3-ylphenyl)-2H-indazole-7-carboxamide; 2-{4-[(3R)-piperidin-3-yl]phenyl}-2H-indazole-7-carboxamide; 2-{4-[(3S)-piperidin-3-yl]phenyl}-2H-indazole-7-carboxamide; 5-fluoro-2-(4-piperidin-3-ylphenyl)-2H-indazole-7-carboxamide; and 5-fluoro-2-{4-[(3S)-piperidin-3-yl]phenyl}-2H-indazole-7-carboxamide, as well as additional compounds.

U.S. Pat. No. 8,058,275 to Xu et al. discloses diazabenzo[de]anthracen-3-one compounds as PARP inhibitors.

U.S. Pat. No. 8,012,976 to Wang et al. discloses dihydropyridophthalazinone compounds as PARP inhibitors, including 5-fluoro-8-(4-fluorophenyl)-9-(1-methyl-1H-1,2,4-triazol-5-yl)-8,9-dihydro-2H-pyrido[4,3,2-de]phthalazin-3(7H)-one.

U.S. Pat. No. 8,008,491 to Jiang et al. discloses substituted aza-indole derivatives as PARP inhibitors, including: 1-phenyl-2-(piperazin-1-yl)-1,3-dihydropyrrolo[2,3-b]pyridine-3-carboxaldehyde, 1-phenyl-2-(piperazin-1-yl)-1H-pyrrolo[2,3-c]pyridine-3-carboxaldehyde, 2-[1,4]diazepan-1-yl-1-phenyl-1H-pyrrolo[2,3-b]pyridine-3-carbaldehyde trifluoroacetic acid salt, and 2-piperazin-1-yl-1-pyridin-3-yl-1H-pyrrolo[2,3-b]pyridine-3-carbaldehyde bis-trifluoroacetic acid salt, as well as additional compounds.

U.S. Pat. No. 7,999,117 to Giranda et al. discloses 1H-benzimidazole-4-carboxamides as PARP inhibitors, including: 6-fluoro-2-[4-((S)-2-hydroxymethylpyrrolidin-1-ylmethyl)phenyl]-1H-benzimidazole-4-carboxamide; 6-fluoro-2-[4-(2-trifluoromethylpyrrolidin-1-ylmethyl)phenyl]-1H-benzimidazole-4-carboxamide; 6-fluoro-2-[4-((R)-2-hydroxymethylpyrrolidin-1-ylmethyl)phenyl]-1H-benzimidazole-4-carboxamide; 2-[4-((S)-2-hydroxymethylpyrrolidin-1-ylmethyl)phenyl]-1H-benzimidazole-4-carboxamide; and 2-[4-(2-trifluoromethylpyrrolidin-1-ylmethyl)phenyl]-1H-benzimidazole-4-carboxamide, as well as additional compounds.

U.S. Pat. No. 7,994,182 to Sumegi et al. discloses quinazolinone derivatives as PARP inhibitors of Formula (PA-IV):

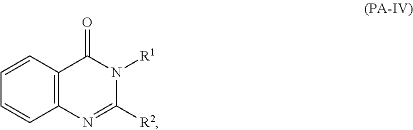

wherein:
(1) R¹ is hydrogen or a moiety of Subformula (PA-IV(a)):

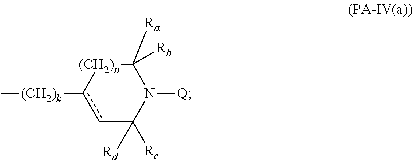

(2) k is 1, 2, 3, or 4;
(3) n is 0 or 1;
(4) Q is an oxyl group or hydrogen;
(5) Rₐ and R_{b} are independently hydrogen or C₁-C₆ alkyl;
(6) R_{b} and R_{d} are independently C₁-C₆ alkyl;
(7) the broken line in the six-membered ring is an optional valence bond (the bond is either a single or a double bond);
(8) R² is either:
(8a) if R¹ is other than hydrogen, hydrogen or C₁-C₆ alkyl;
(8b) if R¹ is hydrogen, a group of Subformula (PA-IV(b)), Subformula (PA-IV(c)), or Subformula (PA-IV(d)):

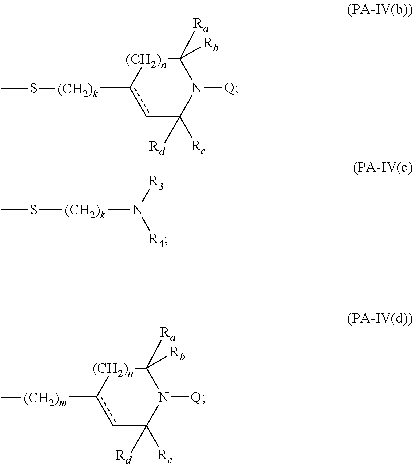

wherein:
(9) in Subformula (PA-IV(b)), k, n, Rₐ, R_{b}, R_{c}, R_{d}, and the broken line are as defined above in (2), (3), (5), (6), and (7);
(10) in Subformula (PA-IV(c)), k is 1, 2, or 3, and R³ and R⁴ are independently C₁-C₆ alkyl;
(11) or together with the attached nitrogen form a group of Subformula (P-IV(e)), wherein p is 0 or 1 and R_{a′}, R_{b′}, R_{c′}, and R_{d′} are independently hydrogen or C₁-C₆ alkyl;

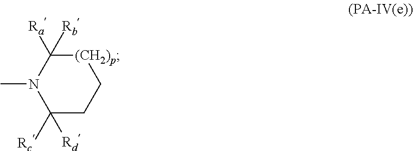

and
(12) in Subformula (P-IV(d), n, Rₐ, R_{b}, R_{c}, R_{d}, and the broken line are as defined above in (3), (5), (6), and (7).

U.S. Pat. No. 7,834,015 to Jones et al. discloses pyrrolo[1,2-a] pyrazin-1(2H)-one and pyrrolo[1,2-d][1,2,4]triazin-1(2H)-one derivatives as PARP inhibitors.

U.S. Pat. No. 7,825,129 to Pellicciari et al. discloses thieno[2,3-c]quinolones as PARP inhibitors, including compounds of Formula (PA-V):

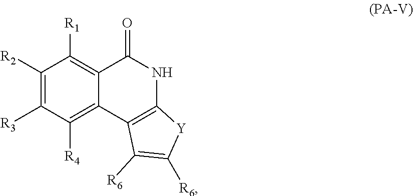

wherein:
(1) Y is selected from sulfur, nitrogen, and oxygen;
(2) R₁, R₂, R₃, R₄, R₅ and R₆ are the same or different, and each represent hydrogen, hydroxy, OR₇, COOR₇, carboxy, amino, NHR₇ or halogen, or R₅ and R₆ taken together form a fused non-aromatic 5- or 6-membered carbocylic ring; and
(3) R₇ is C₁-C₆ alkyl, C₂-C₆ alkenyl or C₃-C₇ cycloalkyl optionally substituted with one or more group selected from hydroxyl, C₁-C₄ alkoxy, carboxy, C₁-C₆ alkoxycarbonyl, amino, C₁-C₆ mono-alkylamino, C₁-C₆ di-alkylamino and halogen.

U.S. Pat. No. 7,820,668 to Xu et al. discloses diazabenzo[de]anthracen-3-one compounds as PARP inhibitors.

U.S. Pat. No. 7,732,491 to Sherman et al. discloses 4-iodo-3-nitrobenzamide as a PARP inhibitor.

U.S. Pat. No. 7,728,026 to Zhu et al. discloses 2-substituted 1H-benzimidazole-4-carboxamides as PARP inhibitors, including 2-(1-amino-1-methylethyl)-1H-benzimidazole-4-carboxamide; 2-[1-methyl-1-(propylamino)ethyl]-1H-benzimidazole-4-carboxamide; 2-[1-(butylamino)-1-methylethyl]-1H-benzimidazole-4-carboxamide; and 2-{1-methyl-1-[(2-phenylethyl)amino]ethyl}-1H-benzimidazole-4-carboxamide, as well as additional compounds.

U.S. Pat. No. 7,595,406 to Penning et al. discloses substituted 1H-benzimidazole-4-carboxamides as PARP inhibitors, including 2-{4-[1-(cyclohexylmethylamino)ethyl]phenyl}-1H-benzimidazole-4-carboxamide; 2-[4-(1-cyclobutylaminoethyl)phenyl]-1H-benzimidazole-4-carboxamide; 2-[3-(2-cyclopropylaminoethyl)phenyl]-1H-benzimidazole-4-carboxamide; and 2-(4-cyclopropylaminomethylphenyl)-1H-benzimidazole-4-carboxamide, as well as additional compounds.

U.S. Pat. No. 7,550,603 to Zhu et al. discloses 1H-benzimidazole-4-carboxamides substituted with a quaternary carbon at the 2-position as PARP inhibitors, including 2-(2-methylpyrrolidin-2-yl)-1H-benzimidazole-4-carboxamide; 2-[(2R)-2-methylpyrrolidin-2-yl]-1H-benzimidazole-4-carboxamide; 2-[(2S)-2-methylpyrrolidin-2-yl]-1H-benzimidazole-4-carboxamide; 2-(1,2-dimethylpyrrolidin-2-yl)-1H-benzimidazole-4-carboxamide; 2-(1-ethyl-2-methylpyrrolidin-2-yl)-1H-benzimidazole-4-carboxamide; and 2-(2-methyl-1-propylpyrrolidin-2-yl)-1H-benzimidazole-4-carboxamide, as well as additional compounds.

U.S. Pat. No. 7,405,300 to Jiang et al. discloses substituted indoles as PARP inhibitors, including 2-(piperazin-1-yl)-1-(3-nitrophenyl)-1H-indole-3-carboxaldehyde; 2-(piperazin-1-yl)-1-(4-methoxyphenyl)-1H-indole-3-carboxaldehyde; 2-(piperazin-1-yl)-1-(4-tert-butylphenyl)-1H-indole-3-carboxaldehyde; 2-(piperazin-1-yl)-1-(4-bromophenyl)-1H-indole-3-carboxaldehyde; and 2-(piperazin-1-yl)-1-(4-chlorophenyl)-1H-indole-3-carboxaldehyde, as well as additional compounds.

U.S. Pat. No. 7,087,637 to Grandel et al. discloses indole derivatives as PARP inhibitors, including: 2-(4(4-n-propyl-piperazin-1-yl)-phenyl)-1H-indol-4-carboxamide; 2-(4-piperazin-1-yl-phenyl)-1H-indol-4-carboxamide; 2-(4(4-isopropyl-piperazin-1-yl)-phenyl)-1H-indol-4-carboxamide; 2-(4(4-benzyl-piperazin-1-yl)-phenyl)-1H-indol-4-carboxamide; 2-(4(4-n-butyl-piperazin-1-yl)-phenyl)-1H-indol-4-carboxamide; and 2-(4(4-ethyl-piperazin-1-yl)-phenyl)-1H-indol-4-carboxamide, as well as additional compounds.

U.S. Pat. No. 7,041,675 to Lubisch et al. discloses substituted pyridine carboxamides as PARP inhibitors, including 2-phenylimidazo[1,2-a]pyridine-8-carboxamide; 2-(4-nitrophenyl)imidazo[1,2-a]pyridine-8-carboxamide; 2-(4-aminophenyl)imidazo[1,2-a]pyridine-8-carboxamide; 2-(2-benzothienyl)imidazo[1,2-a]pyridine-8-carboxamide; 2-(4-bromophenyl)-imidazo[1,2-a]pyridine-8-carboxamide; and 2-(4-imidazol-1-ylphenyl)imidazo[1,2-a]pyridine-8-carboxamide, as well as additional compounds.

U.S. Pat. No. 6,924,284 to Beaton et al. discloses substituted bicyclic aryl PARP inhibitors, including: N-[3-(4-oxo-3,4-dihydro-phthalazin-1-ylamino)-propyl]-3-[3-(1H-pyrrol-2-yl)-[1,2,4]oxadiaol-5-yl]propionamide; N-[3-(4-oxo-3,4-dihydro-phthalazin-1-ylamino)-propyl]-3-(3-thiophen-3-yl-[1,2,4]oxadiazol-5-yl)propionamide; 3-(3-furan-2-yl-[1,2,4]oxadiazol-5-yl)-N-[3-(4-oxo-3,4-dihydro-phthalazin-1-ylamino)-propyl]-propionamide; and N-[3-(4-oxo-3,4-dihydro-phthalazin-1-ylamino)-propyl]-3-(3-thiophen-2-yl-[1,2,4]oxadiazol-5-yl)-propionamide, as well as additional compounds.

U.S. Pat. No. 6,635,642 to Jackson et al. discloses phthalazinone derivatives as PARP inhibitors, including 4-(3-nitro-4-(piperidin-1-yl)phenyl-phthalazin-1(2H)-one; 4-(4-(dimethylamino)-3-nitrophenyl)-phthalazin-1(2H)-one; 4-(3-amino-4-(dimethylamino)phenyl)-phthalazin-1(2H)-one; 4-(4-phenylpiperazin-1-yl)-phthalazin-1(2H)-one; and 4-(4-(4-chlorophenyl)-piperazin-1-yl)-phthalazin-1 (2H)-one, as well as additional compounds.

U.S. Pat. No. 6,448,271 to Lubisch et al. discloses substituted benzimidazoles as PARP inhibitors, including 2-(piperidin-4-yl)benzimidazole-4-carboxamide dihydrochloride; 2-(N-acetylpiperidin-4-yl)benzimidazole-4-carboxamide; 2-(N-propylpiperidin-4-yl)benzimidazole-4-carboxamide; 2-piperidin-3-ylbenzimidazole-4-carboxamide dihydrochloride; and 2-(N—(O-t-butoxycarbonyl)piperidin-3-yl)benzimidazole-4-carboxamide; 2-(N-benzylpiperidin-3-yl)benzimidazole-4-carboxamide, as well as additional compounds.

U.S. Pat. No. 6,426,415 to Jackson et al. discloses alkoxy-substituted PARP inhibitors, including 1-(benzyloxy)-5-methylphthalazine; 1-(methoxy)-5-methyl-phthalazine; 1-(ethoxy)-5-methylphthalazine; 1-(propoxy)-5-methylphthalazine; 1-(butoxy)-5-methyl-phthalazine; 1-(methoxy)-5-hydroxyphthalazine; 1-(ethoxy)-5-hydroxyphthalazine; 1-(propoxyoxy)-5-hydroxy-phthalazine; and 1-(butoxy)-5-hydroxyphthalazine, as well as additional compounds.

U.S. Pat. No. 6,395,749 to Li et al. discloses substituted carboxamides as PARP inhibitors, including 5-carbamoylquinoline-4-carboxylic acid.

U.S. Pat. No. 6,387,902 to Zhang et al. discloses substituted phenazines as PARP inhibitors, including compounds of Formula (PA-VI):

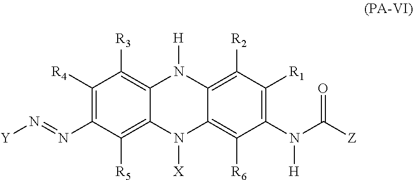

wherein:
(1) R₁-R₉ and Z are independently hydrogen, hydroxy, halo, haloalkyl, thiocarbonyl, cyano, nitro, amino, imino, alkylamino, aminoalkyl, sulfhydryl, thioalkyl, alkylthio, sulfonyl, alkylsulfonyl, C₁-C₉ straight or branched chain alkyl, C₂-C₉ straight or branched chain alkenyl, C₂-C₉ straight or branched chain alkynyl, C₁-C₆ straight or branched chain alkoxy, C₂-C₆ straight or branched chain alkenoxy, C₂-C₆ straight or branched chain alkynoxy, aryl, carbocycle, heterocycle, aralkyl, alkylaryl, alkylaryloxy, aryloxy, aralkyloxy, aralkylsulfonyl, aralkylamino, arylamino, arylazo, arylthio, or aralkylthio; or
(2) Z is a moiety of Subformula (PA-VI(a))

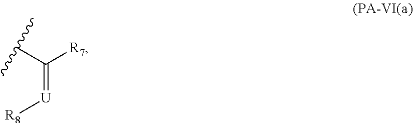

wherein U is C or N; R₇ and R₈ are as defined in (1); and X and Y are independently aryl, carbocycle, or heterocycle.

U.S. Pat. No. 6,380,211 to Jackson et al. discloses alkoxy-substituted PARP inhibitors, including 1-(methoxy)-5-methylisoquinoline, 1-(ethoxy)-5-methyl-isoquinoline, 1-(propoxy)-5-methylisoquinoline, 1-(butoxy)-5-methylisoquinoline, 1-(ethoxy)-5-hydroxy-isoquinoline, 1-(propoxy)-5-hydroxyisoquinoline, 1-(butoxy)-5-hydroxyisoquinoline, 1-(benzyloxy)-5-methylphthalazine and 1-(benzyloxy)-5-methylisoquinoline, as well as additional compounds.

U.S. Pat. No. 6,358,975 to Eliasson et al. discloses PARP inhibitors, including 6(5H)-phenanthridinone, 2-nitro-6(5H)-phenanthridinone, 4-hydroxyquinazoline, 2-methyl-4(3H)-quinazoline, 2-mercapto-4(3H)-quinazoline, benzoyleneurea, 6-amino-1,2-benzopyrone, trp-P-1(3-amino-1,4-dimethyl-5H-pyrido[4,3-b]indole), juglone, luminol, 1(2H)-phthalazinone, phthalhydrazide, and chlorothenoxazin.

U.S. Pat. No. 6,235,748 to Li et al. discloses oxo-substituted compounds containing at least one ring nitrogen as PARP inhibitors.

U.S. Pat. No. 6,201,020 to Zhang et al. discloses ortho-diphenol compounds as PARP inhibitors, including compounds of Formula (PA-VII):

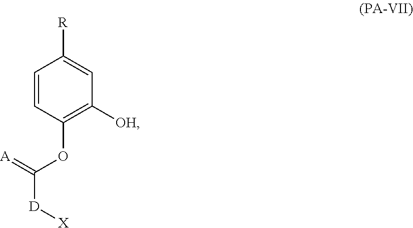

wherein:
(1) A is O or S;
(2) R is C₁-C₁₀ straight or branched chain alkyl, C₂-C₁₀ straight or branched chain alkenyl, C₂-C₁₀ straight or branched chain alkynyl, aryl, heteroaryl, carbocycle, or heterocycle;
(3) D is a bond, or a C₁-C₃ straight or branched chain alkyl, C₂-C₃ straight or branched chain alkenyl, C₂-C₃ straight or branched chain alkynyl, wherein any of the carbon atoms of said alkyl, alkenyl, or alkynyl of D are optionally replaced with oxygen, nitrogen, or sulfur; and
(4) X is aryl, heteroaryl, carbocycle, or heterocycle.

U.S. Pat. No. 5,756,510 to Griffin et al. discloses benzamide analogs that are PARP inhibitors, including: 3-benzyloxybenzamide; 3-(4-methoxybenzyloxy)benzamide; 3-(4-nitrobenzyloxy)benzamide; 3-(4-azidobenzyloxy)benzamide; 3-(4-bromobenzyloxy)benzamide; 3-(4-fluorobenzyloxy)benzamide; 3-(4-aminobenzyloxy)benzamide; 3-(3-nitrobenzyloxy)benzamide; 3-(3,4-methylenedioxyphenylmethyloxy)benzamide; 3-(piperonyloxy)benzamide; 3-(N-acetyl-4-aminobenzyloxy)benzamide; and 3-(4-trifluoromethylbenzyloxy)benzamide; and 3-(4-cyanobenzyloxy)benzamide, as well as additional compounds.

United States Patent Application Publication No. 2015/0175617 by Zhou et al. discloses fused tetra or penta-cyclic dihydrodiazepinocarbazolones as PARP inhibitors, including: 2,3,5,10-tetrahydro-[1,2]diazepino[3,4:5,6-def]carbazol-6(1H)-one; 5,6,7,8-tetrahydro-4H-4,9,10-triazaindeno[2,1,7-kla]heptalen-11(10H)-one; 2-methyl-2,3,5,10-tetrahydro-[1,2]diazepino[3,4:5,6-def]carbazol-6(1H)-one; and 3,3-dimethyl-2,3,5,10-tetrahydro-[1,2]diazepino[3,4:5,6-def]carbazol-6(1H)-one, as well as additional compounds.

United States Patent Application Publication No. 2015/0152118 by Jana et al. discloses tetrahydroquinazolinone derivatives as PARP inhibitors, including: 2′-(3-(4-(4-fluorophenyl)piperazin-1-yl)propyl)-6′,7′-dihydro-3′H-spiro[cyclopropane-1,8′-quinazolin]-4′(5′H)-one; 2′-(3-(4-(4-chlorophenyl)piperazin-1-yl)propyl)-6′,7′-dihydro-3′H-spiro[cyclopropane-1,8′-quinazolin]-4′(5′H)-one; 2′-(3-(4-phenyl-5,6-dihydropyridin-1(2H)-yl)propyl)-6′,7′-dihydro-3′H-spiro[cyclopropane-1,8′-quinazolin]-4′(5′H)-one; and 2′-(3-(3-(4-fluorophenyl)-3,8-diazabicyclo[3.2.1]octan-8-yl)propyl)-4a′,5′,6′,7′-tetrahydro-3′H-spiro[cyclopropane-1,8′-quinazolin]-4′(8a′H)-one, as well as additional compounds.

United States Patent Application Publication No. 2015/0031652 by Gangloff et al. discloses substituted 1,2,3,4-tetrahydropyrido[2,3-b]pyrazines as PARP inhibitors, including (S)-3-((4-(4-chlorophenyl)piperazin-1-yl)methyl)-6a,7,8,9-tetrahydropyrido[3,2-e]pyrrolo[1,2-a]pyrazin-6(5H)-one; (S)-3-((4-(4-chlorophenyl)-5,6-dihydropyridin-1(2H)-yl)methyl)-6a,7,8,9-tetrahydropyrido[3,2-e]pyrrolo[1,2-a]pyrazin-6(5H)-one; (S)-3-((4-(4-chlorophenyl)piperidin-1-yl)methyl)-6a,7,8,9-tetrahydropyrido[3,2-e]pyrrolo[1,2-a]pyrazin-6(5H)-one; and (S)-4-(4-((6-oxo-5,6,6a,7,8,9-hexahydropyrido[3,2-e]pyrrolo[1,2-a]pyrazin-3-yl)methyl)piperazin-1-yl)benzonitrile, as well as additional compounds.

United States Patent Application Publication No. 2015/0025071 by Buchstaller et al. discloses tetrahydroquinazolinone derivatives as PARP inhibitors, including: 2-[4-(4-methoxy-phenyl)-piperazin-1-yl]-5,6,7,8-tetrahydro-3H-quinazolin-4-one; 2-[4-(3-fluorophenyl)piperazin-1-yl]-5,6,7,8-tetrahydro-3H-quinazolin-4-one; 2-[4-(4-fluorophenyl)piperazin-1-yl]-5,6,7,8-tetrahydro-3H-quinazolin-4-one; and 2-[4-(3-methoxyphenyl)piperazin-1-yl]-5,6,7,8-tetrahydro-3H-quinazolin-4-one, as well as additional compounds.

United States Patent Application Publication No. 2015/0018356 by Zhou et al. discloses fused tetra- or pentacyclic pyridophthalazinones as PARP inhibitors.

United States Patent Application Publication No. 2014/0336192 to Papeo et al. discloses substituted 3-phenyl-isoquinolin-1(2H)-one derivatives as PARP inhibitors, including: 4-(2-amino-ethoxy)-3-(4-bromo-phenyl)-7-fluoro-2H-isoquinolin-1-one; 4-(2-amino-ethoxy)-7-fluoro-3-(3-trifluoromethyl-phenyl)-2H-isoquinolin-1-one; 4-(2-amino-ethoxy)-7-fluoro-3-(4-morpholin-4-yl-phenyl)-2H-isoquinolin-1-one; 4-(2-amino-ethoxy)-3-(3-bromo-4-morpholin-4-yl-phenyl)-7-fluoro-2H-isoquinolin-1-one; 4-(2-amino-ethoxy)-3-(3-bromo-phenyl)-7-fluoro-2H-isoquinolin-1-one; and 4-[4-(2-amino-ethoxy)-7-fluoro-1-oxo-1,2-dihydro-isoquinolin-3-yl]-benzonitrile, as well as additional compounds.

United States Patent Application Publication No. 2014/0235675 by Papeo et al. discloses 3-oxo-2,3-dihydro-1H-indazole-4-carboxamide derivatives as PARP inhibitors, including: 3-oxo-2-(piperidin-4-yl)-2,3-dihydro-1H-indazole-4-carboxamide; 2-(1-cyclopentylpiperidin-4-yl)-3-oxo-2,3-dihydro-1H-indazole-4-carboxamide; 2-(1-cyclohexylpiperidin-4-yl)-3-oxo-2,3-dihydro-1H-indazole-4-carboxamide; and 2-[1-(4,4-difluorocyclohexyl)piperidin-4-yl]-3-oxo-2,3-dihydro-1H-indazole-4-carboxamide, as well as additional compounds.

United States Patent Application Publication No. 2014/0023642 by Cai et al. discloses 1-(arylmethyl)quinazoline-2,4(1H,3H)-diones as PARP inhibitors, including: 1-(3-methoxycarbonylbenzyl)quinazoline-2,4(1H,3H)-dione; 1-(3-carboxybenzyl)quinazoline-2,4(1H,3H)-dione; 1-(3-(4-(pyridin-2-yl)piperazine-1-carbonyl)benzyl)quinazoline-2,4(1H,3H)-dione; 1-(3-(4-(pyrimidin-2-yl)piperazine-1-carbonyl)benzyl)quinazoline-2,4(1H,3H)-dione; and 1-(3-(4-cyclohexylpiperazine-1-carbonyl)benzyl)quinazoline-2,4(1H,3H)-dione, as well as additional compounds.

United States Patent Application Publication No. 2013/0225647 by Donawho et al. discloses PARP inhibitors of Formula (PA-VIII):

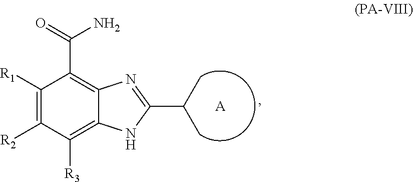

wherein:
(1) R₁, R₂, and R₃ are independently selected from the group consisting of hydrogen, alkenyl, alkoxy, alkoxycarbonyl, alkyl, alkynyl, cyano, haloalkoxy, haloalkyl, halogen, hydroxy, hydroxyalkyl, nitro, NR_{A}R_{B}, and (NR_{A}R_{B})carbonyl;
(2) A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, heterocycle, heterocyclylalkyl, heteroaryl, heteroarylalkyl, hydroxy, hydroxyalkyl, nitro, NR_{C}R_{D}, (NR_{C}R_{D})alkyl, (NR_{C}R_{D})carbonyl, (NR_{C}R_{D})carbonylalkyl, and (NR_{C}R_{D})sulfonyl; and;
(3) R_{A}, R_{B}, R_{C}, and R_{D} are independently selected from the group consisting of hydrogen, alkyl, and alkylcarbonyl.

United States Patent Application Publication No. 2013/0129841 by Ciavolella et al. discloses PARP inhibitors including 2-[1-(4,4-difluorocyclohexyl)piperidin-4-yl]-3-oxo-2,3-dihydro-1H-isoindole-4-carboxamide; 2-[1-(4,4-difluorocyclohexy)piperidin-4-yl]-6-fluoro-3-oxo-2,3-dihydro-1H-isoindole-4-carboxamide; 6-fluoro-3-oxo-2-[1-(4-oxocyclohexy)piperidin-4-yl]-2,3-dihydro-1H-isoindole-4-carboxamide, and 2-[1-(4,4-dichlorocyclohexyl)piperidin-4-yl]-6-fluoro-3-oxo-2,3-dihydro-1-H-isoindole-4 carboxamide, as well as additional compounds.

Other PARP inhibitors are known in the art and can be employed in methods and compositions according to the present invention. These additional PARP inhibitors include, but are not limited to: (1) derivatives of tetracycline as described in U.S. Pat. No. 8,338,477 to Duncan et al.; (2) 3,4-dihydro-5-methyl-1(2H)-isoquinoline, 3-aminobenzamide, 6-aminonicotinamide, and 8-hydroxy-2-methyl-4(3H)-quinazolinone, as described in U.S. Pat. No. 8,324,282 by Gerson et al.; (3) 6-(5H)-phenanthridinone and 1,5-isoquinolinediol, as described in U.S. Pat. No. 8,324,262 by Yuan et al.; (4) (R)-3-[2-(2-hydroxymethylpyrrolidin-1-yl)ethyl]-5-methyl-2H-isoquinolin-1-one, as described in U.S. Pat. No. 8,309,573 to Fujio et al.; (5) 6-alkenyl-substituted 2-quinolinones, 6-phenylalkyl-substituted quinolinones, 6-alkenyl-substituted 2-quinoxalinones, 6-phenylalkyl-substituted 2-quinoxalinones, substituted 6-cyclohexylalkyl substituted 2-quinolinones, 6-cyclohexylalkyl substituted 2-quinoxalinones, substituted pyridones, quinazolinone derivatives, phthalazine derivatives, quinazolinedione derivatives, and substituted 2-alkyl quinazolinone derivatives, as described in U.S. Pat. No. 8,299,256 to Vialard et al.; (6) 5-bromoisoquinoline, as described in U.S. Pat. No. 8,299,088 to Mateucci et al.; (7) 5-bis-(2-chloroethyl)amino]-1-methyl-2-benzimidazolebutyric acid, 4-iodo-3-nitrobenzamide, 8-fluoro-5-(4-((methylamino)methyl)phenyl)-3,4-dihydro-2H-azepino[5,4,3-cd]indol-1(6H)-one phosphoric acid, and N-[3-(3,4-dihydro-4-oxo-1-phthalazinyl)phenyl]-4-morpholinebutanamide methanesulfonate, as described in U.S. Pat. No. 8,227,807 to Gallagher et al.; (8) pyridazinone derivatives, as described in U.S. Pat. No. 8,268,827 to Branca et al.; (9) 4-[3-(4-cyclopropanecarbonyl-piperazine-1-carbonyl)-4-fluorobenzyl]-2H-phthalazin-1-one, as described in U.S. Pat. No. 8,247,416 to Menear et al.; (10) tetraaza phenalen-3-one compounds, as described in U.S. Pat. No. 8,236,802 to Xu et al.; (11) 2-substituted-1H-benzimidazole-4-carboxamides, as described in U.S. Pat. No. 8,217,070 to Zhu et al.; (12) substituted 2-alkyl quinazolinones, as described in U.S. Pat. No. 8,188,103 to Van der Aa et al.; (13) 1H-benzimidazole-4-carboxamides, as described in U.S. Pat. No. 8,183,250 to Penning et al.; (14) indenoisoquinolinone analogs, as described in U.S. Pat. No. 8,119,654 to Jagtap et al.; (15) benzoxazole carboxamides, described in U.S. Pat. No. 8,088,760 to Chu et al; (16) diazabenzo[de] anthracen-3-one compounds, described in U.S. Pat. No. 8,058,075 to Xu et al.; (17) dihydropyridophthalazinones, described in U.S. Pat. No. 8,012,976 to Wang et al., (18) substituted azaindoles, described in U.S. Pat. No. 8,008,491 to Jiang et al.; (19) fused tricyclic compounds, described in U.S. Pat. No. 7,956,064 to Chua et al.; (20) substituted 6a,7,8,9-tetrahydropyrido[3,2-e]pyrrolo[1,2-a]pyrazin-6(5H)-ones, described in U.S. Pat. No. 7,928,105 to Gangloff et al.; and (21) thieno[2,3-c] isoquinolines, described in U.S. Pat. No. 7,825,129, all of which patents are incorporated herein by this reference. Still other PARP inhibitors are known in the art. Additionally, derivatives and analogs of PARP inhibitors described above that have PARP-inhibiting activity that is sufficiently great that they could replace the PARP inhibitors described above in methods or compositions according to the present invention can also be employed in methods and compositions according to the present invention.

When the improvement is made by chemosensitization, the chemosensitization can be, but is not limited to, use of irinotecan, topotecan, or a derivative or analog of irinotecan or topotecan as a chemosensitizer in combination with a therapeutic agent selected from the group consisting of:
(a) fraudulent nucleosides;
(b) fraudulent nucleotides;
(c) thymidylate synthetase inhibitors;
(d) signal transduction inhibitors;
(e) cisplatin or platinum analogs;
(f) alkylating agents such as BCNU, Gliadel wafers, CCNU, bendamustine (Treanda), or temozolomide (Temodar);
(g) anti-tubulin agents;
(h) antimetabolites;
(i) berberine;
(j) apigenin;
(k) amonafide;
(l) colchicine or analogs of colchicine;
(m) genistein;
(n) etoposide;
(o) cytarabine;
(p) vinca alkaloids;
(q) 5-fluorouracil;
(r) curcumin;
(s) NF-κB inhibitors;
(t) rosmarinic acid;
(u) dianhydrogalactitol;
(v) dibromodulcitol;
(w) biological therapies such as antibodies such as Avastin, Rituxan, Herceptin, Erbitux, PD-1 and PDL-1 inhibitors;
(x) prednimustine;
(y) DNA and RNA therapeutics;
(z) Braf inhibitors;
(aa) BTK inhibitors;
(ab) 5-azacytidine;
(ac) decitabine;
(ad) PARP inhibitors;
(ae) hypomethylating agents;
(af) histone deacetylase inhibitors; and
(ag) vincristine.

When the improvement is made by chemopotentiation, the chemopotentiation can be, but is not limited to, use of irinotecan, topotecan, or a derivative or analog of irinotecan or topotecan as a chemopotentiator in combination with a therapeutic agent selected from the group consisting of:
(a) fraudulent nucleosides;
(b) fraudulent nucleotides;
(c) thymidylate synthetase inhibitors;
(d) signal transduction inhibitors;
(e) cisplatin or platinum analogs;
(f) alkylating agents such as BCNU, Gliadel wafers, CCNU, bendamustine (Treanda), or temozolomide (Temodar);
(g) anti-tubulin agents;
(h) antimetabolites;
(i) berberine;
(j) apigenin;
(k) amonafide;
(l) colchicine or analogs of colchicine;
(m) genistein;
(n) etoposide;
(o) cytarabine;
(p) vinca alkaloids;
(q) 5-fluorouracil;
(r) curcumin;
(s) NF-κB inhibitors;
(t) rosmarinic acid;
(u) dianhydrogalactitol;
(v) dibromodulcitol;
(w) biological therapies such as antibodies such as Avastin, Rituxan, Herceptin, Erbitux, PD-1 and PDL-1 inhibitors;
(x) prednimustine;
(y) DNA and RNA therapeutics;
(z) Braf inhibitors;
(aa) BTK inhibitors;
(ab) 5-azacytidine;
(ac) decitabine;
(ad) PARP inhibitors;
(ae) hypomethylating agents;
(af) histone deacetylase inhibitors; and
(ag) vincristine.

When the improvement is made by post-treatment management, the post-treatment management can be, but is not limited to, a method for post-treatment management selected from the group consisting of:
(a) use with therapies associated with pain management;
(b) nutritional support;
(c) anti-emetics;
(d) anti-nausea therapies;
(e) anti-anemia therapy;
(f) anti-inflammatories;
(g) antipyretics;
(h) immune stimulants;
(i) anti diarrhea medicines;
(j) famotidine;
(k) antihistamines;
(l) suppository lubricants;
(m) soothing agents;
(n) lidocaine; and
(o) hydrocortisone.

When the improvement is made by alternative medicine/therapeutic support, the alternative medicine/therapeutic support can be, but is not limited to, a method for alternative medicine/therapeutic support selected from the group consisting of:
(a) NF-κB inhibitors;
(b) natural anti-inflammatories;
(c) immunostimulants; and
(d) flavonoids or flavones.

Flavonoids and flavones include, but are not limited to, apigenin, genistein, apigenenin, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, or 6-O-acetylglycitin.

When the improvement is made by bulk drug product improvements, the bulk drug product improvement can be, but is not limited to, a bulk drug product improvement selected from the group consisting of:
(a) salt formation;
(b) homogenous crystalline structure;
(c) pure isomers, such as stereoisomers;
(d) increased purity;
(e) lower residual solvents; and
(f) lower residual heavy metals.

When the improvement is made by diluent systems, the diluent system can be, but is not limited to, a diluent system selected from the group consisting of:
(a) emulsions;
(b) dimethyl sulfoxide (DMSO);
(c) N-methyl formamide (NMF);
(d) dimethylformamide (DMF);
(e) dimethylacetamide (DMA);
(f) ethanol;
(g) benzyl alcohol;
(h) dextrose containing water for injection;
(i) Cremophor;
(j) cyclodextrins;
(k) PEG;
(l) agents to sweeten such as saccharin, sucralose, or aspartame;
(m) agents to thicken an oral dosage form such as glycerin;
(n) taste-masking effectors such as menthol, rum flavor fruit flavorings, or chocolate; and
(o) buffers to yield a pH value as buffered of less than 4.

When the improvement is made by solvent systems, the solvent system can be, but is not limited to, a solvent system selected from the group consisting of:
(a) emulsions;
(b) DMSO;
(c) NMF;
(d) DMF;
(e) DMA;
(f) ethanol;
(g) benzyl alcohol;
(h) dextrose-containing water for injection;
(i) Cremophor;
(j) PEG;
(k) glycerin; and
(l) cocoa butter for suppositories.

When the improvement for use is excipients, the excipient can be, but is not limited to, an excipient selected from the group consisting of:
(a) mannitol;
(b) albumin;
(c) EDTA;
(d) sodium bisulfite;
(e) benzyl alcohol;
(f) carbonate buffers;
(g) phosphate buffers;
(h) benzoate preservatives;
(i) glycerin;
(j) sweeteners;
(k) taste-masking agents such as rum flavor;
(m) menthol substituted celluloses;
(n) sodium azide as a preservative; and
(o) flavors for oral dosage forms.

When the improvement is made by use of a dosage form, the dosage form can be, but is not limited to, a dosage form selected from the group consisting of:
(a) liquid in gel capsules;
(b) tablets;
(c) capsules;
(d) topical gels;
(e) topical creams;
(f) patches;
(g) suppositories;
(h) lyophilized dosage fills;
(i) suppositories with quick release (<15 minutes) or long melt times (>15 minutes) leading to extended release time;
(j) temperature-adjusted suppositories;
(k) oral solutions; and
(l) suspensions of varying concentrations of active therapeutic agent or prodrug, such as 1-100 mg/mL.

Formulation of pharmaceutical compositions in tablets, capsules, and topical gels, topical creams or suppositories is well known in the art and is described, for example, in United States Patent Application Publication No. 2004/0023290 by Griffin et al. Formulation of pharmaceutical compositions as patches such as transdermal patches is well known in the art and is described, for example, in U.S. Pat. No. 7,728,042 to Eros et al.

Lyophilized dosage fills are also well known in the art. One general method for the preparation of such lyophilized dosage fills, applicable to many therapeutic agents, comprises the following steps:
(1) Dissolve the drug in water for injection precooled to below 10° C. Dilute to final volume with cold water for injection to yield a 40 mg/mL solution.
(2) Filter the bulk solution through an 0.2-μm filter into a receiving container under aseptic conditions. The formulation and filtration should be completed in 1 hour.
(3) Fill nominal 1.0 mL filtered solution into sterilized glass vials in a controlled target range under aseptic conditions.
(4) After the filling, all vials are placed with rubber stoppers inserted in the “lyophilization position” and loaded in the prechilled lyophilizer. For the lyophilizer, shelf temperature is set at +5° C. and held for 1 hour; shelf temperature is then adjusted to −5° C. and held for one hour, and the condenser, set to −60° C., turned on.
(5) The vials are then frozen to 30° C. or below and held for no less than 3 hours, typically 4 hours.
(6) Vacuum is then turned on, the shelf temperature is adjusted to −5° C., and primary drying is performed for 8 hours; the shelf temperature is again adjusted to −5° C. and drying is carried out for at least 5 hours.
(7) Secondary drying is started after the condenser (set at −60° C.) and vacuum are turned on. In secondary drying, the shelf temperature is controlled at +5° C. for 1 to 3 hours, typically 1.5 hours, then at 25° C. for 1 to 3 hours, typically 1.5 hours, and finally at 35-40° C. for at least 5 hours, typically for 9 hours, or until the product is completely dried.
(8) Break the vacuum with filtered inert gas (e.g., nitrogen). Stopper the vials in the lyophilizer.
(9) Vials are removed from the lyophilizer chamber and sealed with aluminum flip-off seals. All vials are visually inspected and labeled with approved labels.

When the improvement is made by dosage kits and packaging, the dosage kits and packaging can be, but are not limited to, dosage kits and packaging selected from the group consisting of:
(a) amber vials to protect from light;
(b) stoppers with specialized coatings to improve shelf-life stability;
(c) specialized dropper measuring devices;
(d) single-use or multiple-use container closure systems;
(e) dosage forms suitable for testing for allergies;
(f) suppository delivery devices;
(g) epinephrine pens for side effect management;
(h) physician and nurse assistance gloves;
(i) measuring devices;
(j) metered syringes;
(k) dosage cups configured to deliver defined doses; and
(l) two-component oral solution systems where therapeutic is added to an oral diluent.

When the improvement is drug delivery systems, the drug delivery system can be, but is not limited to, a drug delivery system selected from the group consisting of:
(a) nanocrystals;
(b) bioerodible polymers;
(c) liposomes;
(d) slow-release injectable gels;
(e) microspheres;
(f) suspensions with glycerin;
(g) meltable drug release suppositories with polymers such as cocoa butter alone or in combination with PEG, lecithin, or polylactide/polyglycolide;
(h) rectal plugs for drug delivery;
(i) micro- or nano-emulsions;
(j) cyclodextrins; and
(k) topical delivery systems.

Nanocrystals are described in U.S. Pat. No. 7,101,576 to Hovey et al.

Bioerodible polymers, also known as biodegradable polymers, are disclosed in N. Kamaly et al., “Degradable Controlled-Release Polymers and Polymeric Nanoparticles: Mechanisms of Controlling Drug Release,” Chem. Rev. 116: 2602-2663 (2016). Bioerodible polymers are also described in U.S. Pat. No. 7,318,931 to Okumu et al. A bioerodible polymer decomposes when placed inside an organism, as measured by a decline in the molecular weight of the polymer over time. Polymer molecular weights can be determined by a variety of methods including size exclusion chromatography (SEC), and are generally expressed as weight averages or number averages. A polymer is bioerodible if, when in phosphate buffered saline (PBS) of pH 7.4 and a temperature of 37° C., its weight-average molecular weight is reduced by at least 25% over a period of 6 months as measured by SEC. Useful bioerodible polymers include polyesters, such as poly(caprolactone), poly(glycolic acid), poly(lactic acid), and poly(hydroxybutryate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

Liposomes are well known as drug delivery vehicles. Liposome preparation is described in European Patent Application Publication No. EP 1332755 by Weng et al. Liposomes can incorporate short oligopeptide sequences capable of targeting the EGFR receptor, as described in United States Patent Application Publication 2012/0213844 by Huang et al. Alternatively, liposomes can include nuclear localization signal/fusogenic peptide conjugates and form targeted liposome complexes, as described in United States Patent Application Publication 2012/0183596 by Boulikas. Additional liposome formulations suitable for use with irinotecan, topotecan, and derivatives and analogs thereof are described herein.

The use of microspheres for drug delivery is known in the art and is described, for example, in H. Okada & H. Taguchi, “Biodegradable Microspheres in Drug Delivery,” Crit. Rev. Ther. Drug Carrier Sys. 12: 1-99 (1995).

When the improvement is made by drug conjugate forms, the drug conjugate form can be, but is not limited to, a drug conjugate form selected from the group consisting of:
(a) polyethylene glycols;
(b) polylactides;
(c) polyglycolides;
(d) amino acids;
(e) peptides; and
(f) multivalent linkers.

Polylactide conjugates are well known in the art and are described, for example, in R. Tong & C. Cheng, “Controlled Synthesis of Camptothecin-Polylactide Conjugates and Nanoconjugates,” Bioconjugate Chem. 21: 111-121 (2010).

Polyglycolide conjugates are also well known in the art and are described, for example, in PCT Patent Application Publication No. WO 2003/070823 by Elmaleh et al.

Multivalent linkers are known in the art and are described, for example, in United States Patent Application Publication No. 2007/0207952 by Silva et al. For example, multivalent linkers can contain a thiophilic group for reaction with a reactive cysteine, and multiple nucleophilic groups (such as NH₂ or OH) or electrophilic groups (such as activated esters) that permit attachment of a plurality of biologically active moieties to the linker.

Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the 8-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the 8-amino group of the side chain of lysine. Many other electrophilic reagents are known that will react with the 8-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154. Furthermore, electrophilic reagents are known that will react with hydroxyl groups such as those in the side chains of serine and threonine, including reactive haloalkane derivatives. These are described in G. T. Hermanson, “Bioconjugate Techniques” (Academic Press, San Diego, 1996), pp. 154-158. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail.

Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996).

When the improvement is made by compound analogs, the compound analog can be, but is not limited to a compound analog selected from the group consisting of:
(a) alteration of side chains to increase or decrease lipophilicity;
(b) additional chemical functionalities to alter reactivity, electron affinity, or binding capacity; and
(c) preparation of salt forms.

When the improvement is made by prodrug systems, the prodrug system can be, but is not limited to a prodrug system selected from the group consisting of:
(a) enzyme sensitive esters;
(b) dimers;
(c) Schiff bases;
(d) pyridoxal complexes;
(e) caffeine complexes;
(f) gastrointestinal system transporters; and
(g) permeation enhancers.

As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24), incorporated herein by this reference. A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987). Exemplary advantages of a prodrug can include, but are not limited to, its physical properties, such as enhanced water solubility for parenteral administration at physiological pH compared to the parent compound, enhanced absorption from the digestive tract, or enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl, C(Y⁴)Y⁵ wherein Y⁴ is H or methyl and Y⁵ is mono-N or di-N,N(C₁-C₆)alkylamino, morpholino, piperidin-1-yl or pyrrolidin-1-yl.

The use of prodrug systems is described in T. Järvinen et al., “Design and Pharmaceutical Applications of Prodrugs” in Drug Discovery Handbook (S. C. Gad, ed., Wiley-Interscience, Hoboken, N J, 2005), ch. 17, pp. 733-796. This publication describes the use of enzyme sensitive esters as prodrugs. The use of dimers as prodrugs is described in U.S. Pat. No. 7,879,896 to Allegretti et al. The use of peptides in prodrugs is described in S. Prasad et al., “Delivering Multiple Anticancer Peptides as a Single Prodrug Using Lysyl-Lysine as a Facile Linker,” J. Peptide Sci. 13: 458-467 (2007). The use of Schiff bases as prodrugs is described in U.S. Pat. No. 7,619,005 to Epstein et al. The use of caffeine complexes as prodrugs is described in U.S. Pat. No. 6,443,898 to Unger et al. The use of nitric oxide-releasing prodrugs is described in N. Nath et al., “JS-K, a Nitric Oxide-Releasing Prodrug, Modulates β-Catenin/TCF Signaling in Leukemic Jurkat Cells: Evidence of an S-Nitrosylated Mechanism,” Biochem. Pharmacol. 80: 1641-1649 (2010). The use of prodrugs that are subject to redox activation is described in S. H. van Rijt & P. J. Sadler, “Current Applications and Future Potential for Bioinorganic Chemistry in the Development of Anticancer Drugs,” Drug Discov. Today 14: 1089-1097 (2009).

Gastrointestinal system transporters are described in J. Xie et al., “Solute Carrier Transporters: Potential Targets for Digestive System Neoplasms,” Cancer Management Res. 10: 153-156 (2018).

Permeation enhancers are described in S. Maher et al., Application of Permeation Enhancers in Oral Delivery of Macromolecules: An Update,” Pharmaceutics 11: 41 (2019) and in A. Kovecik et al., “Permeation Enhancers in Transdermal Drug Delivery: Benefits and Limitations,” Exp. Opin. Drug Deliv. 17: 145-155 (2020).

When the improvement is made by multiple drug systems, the multiple drug system can be, but is not limited to a multiple drug system selected from the group consisting of:
(a) inhibitors of multi-drug resistance;
(b) specific drug resistance inhibitors;
(c) specific inhibitors of selective enzymes;
(d) signal transduction inhibitors;
(e) repair inhibition;
(f) topoisomerase inhibitors with non-overlapping side effects;
(g) multiple agents with different therapeutic mechanisms as in MIME chemotherapy for Hodgkin's disease;
(h) temozolomide;
(i) substituted hexitols;
(j) cephalosporin antibiotics;
(k) caffeine; and
(l) PARP inhibitors.

Multi-drug resistance inhibitors are described in U.S. Pat. No. 6,011,069 to Inomata et al.

Specific drug resistance inhibitors are described in T. Hideshima et al., “The Proteasome Inhibitor PS-341 Inhibits Growth, Induces Apoptosis, and Overcomes Drug Resistance in Human Multiple Myeloma Cells,” Cancer Res. 61: 3071-3076 (2001).

Selective inhibitors of specific enzymes are described in D. Leung et al., “Discovering Potent and Selective Reversible Inhibitors of Enzymes in Complex Proteomes,” Nature Biotechnol. 21: 687-691 (2003).

Repair inhibition is described in N. M. Martin, “DNA Repair Inhibition and Cancer Therapy,” J. Photochem. Photobiol. B 63: 162-170 (2001).

When the improvement is made by biotherapeutic enhancement, the biotherapeutic enhancement can be, but is not limited to, the use of irinotecan, topotecan, or a derivative or analog of irinotecan or topotecan in combination as sensitizers/potentiators with biological response modifiers, wherein the biological response modifier is selected from the group consisting of:
(a) cytokines;
(b) lymphokines;
(c) therapeutic antibodies such as Avastin, Herceptin, Rituxan, and Erbitux;
(d) antisense therapies;
(e) gene therapies;
(f) ribozymes;
(g) RNA interference; and
(h) cell-based therapeutics such as CAR-T.

Antisense therapies are described, for example, in B. Weiss et al., “Antisense RNA Gene Therapy for Studying and Modulating Biological Processes,” Cell. Mol. Life Sci. 55: 334-358 (1999).

Ribozymes are described, for example, in S. Pascolo, “RNA-Based Therapies” in Drug Discovery Handbook (S. C. Gad, ed., Wiley-Interscience, Hoboken, N J, 2005), ch. 27, pp. 1273-1278.

RNA interference is described, for example, in S. Pascolo, “RNA-Based Therapies” in Drug Discovery Handbook (S. C. Gad, ed., Wiley-Interscience, Hoboken, N J, 2005), ch. 27, pp. 1278-1283.

CAR-T therapeutics are described in H. Zhang et al., “Engineering Better Chimeric Antigen Receptor T Cells,” Exp. Hematol. Oncol. 9: 34 (2020) and in S. Srivastava & S. R. Riddell, “Engineering CAR-T Cells: Design Concepts,” Trends Immunol. 36: 494-502 (2015).

When the improvement is made by biotherapeutic resistance modulation, the biotherapeutic resistance modulation can be, but is not limited to, use of irinotecan, topotecan, or a derivative or analog of irinotecan or topotecan to overcome developing or complete resistance to a biotherapeutic agent for tumor treatment, wherein the biotherapeutic agent is selected from the group consisting of:
(a) biological response modifiers;
(b) cytokines;
(c) lymphokines;
(d) therapeutic antibodies such as Avastin, Rituxan, Herceptin, Erbitux;
(e) antisense therapies;
(f) gene therapies:
(g) ribozymes;
(h) RNA interference; and
(i) CAR-T therapies.

When the improvement is made by radiation therapy enhancement, the radiation therapy enhancement can be, but is not limited to, the use of irinotecan, topotecan, or a derivative or analog of irinotecan or topotecan in combination with ionizing radiation, phototherapies, heat therapies, or radio-frequency generated therapies selected from the group consisting of:
(a) use with hypoxic cell sensitizers;
(b) use with radiation sensitizers/protectors;
(c) use with photosensitizers;
(d) use with radiation repair inhibitors;
(e) use with agents for thiol depletion;
(f) use with vaso-targeted agents;
(g) use with radioactive seeds;
(h) use with radionuclides;
(i) use with radiolabeled antibodies; and
(j) use with brachytherapy.

Hypoxic cell sensitizers are described in C. C. Ling et al., “The Effect of Hypoxic Cell Sensitizers at Different Irradiation Dose Rates,” Radiation Res. 109: 396-406 (1987). Radiation sensitizers are described in T. S. Lawrence, “Radiation Sensitizers and Targeted Therapies,” Oncology 17 (Suppl. 13) 23-28 (2003). Radiation protectors are described in S. B. Vuyyuri et al., “Evaluation of D-Methionine as a Novel Oral Radiation Protector for Prevention of Mucositis,” Clin. Cancer Res. 14: 2161-2170 (2008). Photosensitizers are described in R. R. Allison & C. H. Sibata, “Oncologic Photodynamic Therapy Photosensitizers: A Clinical Review,” Photodiagnosis Photodynamic Ther. 7: 61-75 (2010). Radiation repair inhibitors and DNA repair inhibitors are described in M. Hingorani et al., “Evaluation of Repair of Radiation-Induced DNA Damage Enhances Expression from Replication-Defective Adenoviral Vectors,” Cancer Res. 68: 9771-9778 (2008). Thiol depleters are described in K. D. Held et al., “Postirradiation Sensitization of Mammalian Cells by the Thiol-Depleting Agent Dimethyl Fumarate,” Radiation Res. 127: 75-80 (1991). Vaso-targeted agents are described in A. L. Seynhaeve et al., “Tumor Necrosis Factor α Mediates Homogeneous Distribution of Liposomes in Murine Melanoma that Contributes to a Better Tumor Response,” Cancer Res. 67: 9455-9462 (2007).

When the improvement is made by novel mechanisms of action, the novel mechanism of action can be, but is not limited to, a novel mechanism of action selected from the group consisting of:
(a) inhibitors of poly-ADP ribose polymerase (PARP);
(b) agents that affect vasculature;
(c) agents that affect vasodilation;
(d) oncogenic targeted agents;
(e) signal transduction inhibitors;
(f) EGFR inhibitors;
(g) protein kinase C inhibitors;
(h) phospholipase C downregulating agents;
(i) jun downregulating agents;
(j) downregulating agents for histone genes,
(k) downregulating agents for VEGF,
(l) agents that modulate the activity of ornithine decarboxylase;
(m) agents that modulate the activity of jun D;
(n) agents that modulate the activity of v-jun;
(o) agents that modulate the activity of GPCRs;
(p) agents that modulate the activity of protein kinase A;
(q) agents that modulate the activity of telomerase;
(r) agents that modulate the activity of prostate specific genes;
(s) agents that modulate the activity of protein kinases; and
(t) agents that modulate the activity of histone deacetylase.

EGFR inhibition is described in G. Giaccone & J. A. Rodriguez, “EGFR Inhibitors: What Have We Learned from the Treatment of Lung Cancer,” Nat. Clin. Pract. Oncol. 11: 554-561 (2005). Protein kinase C inhibition is described in H. C. Swannie & S. B. Kaye, “Protein Kinase C Inhibitors,” Curr. Oncol. Rep. 4: 37-46 (2002). Phospholipase C downregulation is described in A. M. Martelli et al., “Phosphoinositide Signaling in Nuclei of Friend Cells: Phospholipase C p Downregulation Is Related to Cell Differentiation,” Cancer Res. 54: 2536-2540 (1994). Downregulation of Jun (specifically, c-Jun) is described in A. A. P. Zada et al., “Downregulation of c-Jun Expression and Cell Cycle Regulatory Molecules in Acute Myeloid Leukemia Cells Upon CD44 Ligation,” Oncogene 22: 2296-2308 (2003). The role of histone genes as a target for therapeutic intervention is described in B. Calabretta et al., “Altered Expression of G1-Specific Genes in Human Malignant Myeloid Cells,” Proc. Natl. Acad. Sci. USA 83: 1495-1498 (1986). The role of VEGF as a target for therapeutic intervention is described in A. Zielke et al., “VEGF-Mediated Angiogenesis of Human Pheochromocytomas Is Associated to Malignancy and Inhibited by anti-VEGF Antibodies in Experimental Tumors,” Surgery 132: 1056-1063 (2002). The role of ornithine decarboxylase as a target for therapeutic intervention is described in J. A. Nilsson et al., “Targeting Ornithine Decarboxylase in Myc-Induced Lymphomagenesis Prevents Tumor Formation,” Cancer Cell 7: 433-444 (2005). The role of ubiquitin C as a target for therapeutic intervention is described in C. Aghajanian et al., “A Phase I Trial of the Novel Proteasome Inhibitor PS341 in Advanced Solid Tumor Malignancies,” Clin. Cancer Res. 8: 2505-2511 (2002). The role of Jun D as a target for therapeutic intervention is described in M. M. Caffarel et al., “JunD Is Involved in the Antiproliferative Effect of Δ⁹-Tetrahydrocannibinol on Human Breast Cancer Cells,” Oncogene 27: 5033-5044 (2008). The role of v-Jun as a target for therapeutic intervention is described in M. Gao et al., “Differential and Antagonistic Effects of v-Jun and c-Jun,” Cancer Res. 56: 4229-4235 (1996). The role of protein kinase A as a target for therapeutic intervention is described in P. C. Gordge et al., “Elevation of Protein Kinase A and Protein Kinase C in Malignant as Compared With Normal Breast Tissue,” Eur. J. Cancer 12: 2120-2126 (1996). The role of telomerase as a target for therapeutic intervention is described in E. K. Parkinson et al., “Telomerase as a Novel and Potentially Selective Target for Cancer Chemotherapy,” Ann. Med. 35: 466-475 (2003). The role of histone deacetylase as a target for therapeutic intervention is described in A. Melnick & J. D. Licht, “Histone Deacetylases as Therapeutic Targets in Hematologic Malignancies,” Curr. Opin. Hematol. 9: 322-332 (2002).

When the improvement is made by selective target cell population therapeutics, the selective target cell population therapeutics can be, but is not limited to, selective target cell population therapeutics selected from the group consisting of:
(a) use against radiation sensitive cells;
(b) use against radiation resistant cells;
(c) use against energy depleted cells; and
(d) use against endothelial cells.

When the improvement is made by use of liposomes for drug delivery, the liposome can be, but is not limited to, a liposomal formulation for the delivery of irinotecan, topotecan, or a derivative or analog thereof selected from the group consisting of:
(a) a liposomal formulation comprising a first liposome-forming material comprising cardiolipin and a second liposome-forming material, wherein the composition comprises from about 1 weight percent to about 50 weight percent irinotecan, about 1 weight percent to about 95 weight percent of phosphatidylcholine, and about 0.001 to about 5 weight percent of α-tocopherol for the delivery of irinotecan;
(b) a liposomal formulation wherein the liposome comprises a liposome formed by a membrane of a lipid bilayer containing a phospholipid as a membrane component, wherein only the outer surface of the liposome is modified with a surface-modifying agent containing a polyethylene glycol, in which irinotecan and/or a salt thereof is encapsulated at a concentration of at least 0.1 mol/mol (drug mol/membrane total lipid mol) by an ion gradient between an inner aqueous phase and an outer aqueous phase of the liposome for the delivery of irinotecan;
(c) a liposome comprising irinotecan or irinotecan hydrochloride, neutral phospholipid, and cholesterol, wherein the weight ratio of the cholesterol to the neutral phospholipid is about 1:3 to about 1:5, and in which the liposome can comprise irinotecan hydrochloride, hydrogenated soybean phosphatidylcholine, polyethylene glycol 2000-distearoyl phosphatidyl ethanolamine, cholesterol, and ethylenediaminetetraacetic acid disodium, wherein the weight ratio of the cholesterol to the hydrogenated soybean phosphatidylcholine is about 1:4 for the delivery of irinotecan;
(d) a liposomal formulation comprising irinotecan sucrose octasulfate 1,2-distearoyl-sn-glycero-3-phosphocholine (DSPC), cholesterol, and a N-(carbonylmethoxypolyethylene glycol-2000)-1,2-distearoyl-sn-glycero-3-phosphoethanolamine for the delivery of irinotecan;
(e) a liposomal formulation wherein the interior of the liposome includes a substituted ammonium moiety of Formula (AM-I):

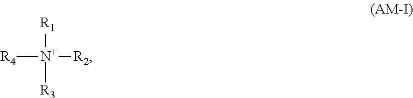

wherein each of R₁, R₂, R₃, and R₄ is independently a hydrogen or an organic group having, inclusively, in totality up to 18 carbon atoms, wherein at least one of R₁, R₂, R₃, and R₄ is an organic group, wherein the organic group is independently a hydrocarbon group having up to 8 carbon atoms, and is an alkyl, alkylidene, heterocyclic alkyl, cycloalkyl, aryl, alkenyl, or cycloalkenyl group or a hydroxy-substituted derivative thereof, optionally including within its hydrocarbon chain a S, O, or N atom, forming an ether, ester, thioether, amine, or amide bond, wherein at least three of R₁, R₂, R₃, and R₄ are organic groups, or the substituted ammonium is a sterically hindered ammonium, such as, for example, where at least one of the organic groups has a secondary or tertiary carbon atom directly linked to the ammonium nitrogen atom for the delivery of irinotecan;
(f) a liposomal formulation wherein the inner space of the liposome contains a polyanion and wherein the polyanion is a polyanionized polyol or a polyanionized sugar, in which suitable substituted ammonium compounds include isopropylethylammonium, isopropylmethylammonium, diisopropylammonium, t-butylethylammonium, dicychohexylammonium, protonized forms of morpholine, pyridine, piperidine, pyrrolidine, piperazine, t-butylamine, 2-amino-2-methylpropanol-1,2-amino-2-methyl-propandiol-1,3, tris-(hydroxyethyl)-aminomethane, trimethylammonium, triethylammonium, tributyl ammonium, diethylmethylammonium, diisopropylethyl ammonium, triisopropylammonium, N-methylmorpholinium, N-hydroxyethylpiperidinium, N-methylpyrrolidinium, N,N′-dimethylpiperazinium, tetramethylammonium, tetraethylammonium, and tetrabutylammonium, and in which the membrane of the liposome can constitute a polymer-conjugated ligand for delivery of irinotecan;
(g) a liposomal formulation wherein the liposome comprises cardiolipin and a second liposome-forming material that is a lipid selected from the group consisting of phosphatidylcholine, cholesterol, α-tocopherol, dipalmitoyl phosphatidylcholine and phosphatidylserine for delivery of irinotecan;
(h) a liposomal formulation wherein the lipid phase comprises cardiolipin and at least one additional lipid component selected from the group consisting of phosphatidylcholine, phosphatidylethanolamine, phosphatidylserine, phosphatidylglycerol, phosphatidic acid, phosphatidylinositol, sphingomyelin, sterol, tocopherol, fatty acid, and mixtures thereof for delivery of irinotecan;
(i) a liposomal formulation wherein the liposomal composition comprises comprising irinotecan sucrose octasulfate (SOS) encapsulated in liposomes comprising one or more phospholipids with a ratio corresponding to a total of 500 grams irinotecan moiety (±10% by weight) per mol total phospholipids, the liposomal irinotecan composition stabilized to have less than 20 mol % (with respect to total phospholipids) lysophosphatidylcholine during the first 6 months of storage of the liposomal irinotecan composition at about 4° C. for delivery of irinotecan; and
(j) a liposomal formulation suspension having selected liposome sizes in the size range between 0.05 and 0.25 μm, and between about 85%-100% liposome-entrapped topotecan, wherein the liposomes can further comprise a cryoprotectant such as sucrose, trehalose, lactose, maltose, cyclodextrin, polyethylene glycol, dextran, polyvinylpyrrolidone, and hydroxyethyl starch, and can comprise lipids such as cholesterol, phosphatidylcholines, sphingomyelins, phosphatidylglycerols, phosphatidic acids, phosphatidylethanolamines, phosphatidylinositols, phosphatidylserines, cholesterol sulfate, or cholesterol hemisuccinate; the lipid used may be conjugated to a hydrophilic polymer such as polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and polyglycerol for the delivery of topotecan.

When the improvement is made by use of a crystalline polymorph, the crystalline polymorph can be, but is not limited to, a crystalline polymorph of irinotecan, topotecan, or a derivative or analog of irinotecan or topotecan selected from the group consisting of:
(a) a crystalline polymorph of irinotecan hydrochloride having a powder X-ray diffraction pattern with 20 peaks at 20.3956 degrees, 22.2950 degrees, 12.0744 degrees, 8.4800 degrees, and 11.8306 degrees;
(b) a crystalline polymorph of irinotecan hydrochloride having a powder X-ray diffraction pattern with 20 peaks at 23.9600 degrees, 20.9200 degrees, and 21.0800 degrees;
(c) a crystalline polymorph of irinotecan hydrochloride having a powder X-ray diffraction pattern with 20 peaks at 12.3406 degrees, 24.7913 degrees, 10.9438 degrees, 8.2056 degrees, 27.6750 degrees, 22.7206 degrees, and 21.2350 degrees;
(d) a crystalline polymorph of irinotecan hydrochloride having a powder X-ray diffraction pattern with 20 peaks at 9.1912 degrees, 9.9800 degrees, 18.8937 degrees, 15.2725 degrees, 16.1681 degrees, 25.7400 degrees, and 27.0662 degrees;
(e) a crystalline polymorph of irinotecan hydrochloride having a powder X-ray diffraction pattern with 20 peaks at 9.15 degrees, 10.00 degrees, 11.80 degrees, 12.20 degrees, 13.00 degrees, and 13.40 degrees;
(f) a crystalline polymorph of irinotecan hydrochloride having a powder X-ray diffraction pattern with 20 peaks at 8.5300 degrees, 9.0400 degrees, 10.23 degrees, 11.65 degrees, 17.01 degrees, 18.08 degrees, 19.17 degrees, and 24.30 degrees;
(g) a crystalline polymorph of irinotecan free base having a powder X-ray diffraction pattern with 20 peaks at 8.70 degrees, 13.10 degrees, 14.50 degrees, 17.40 degrees, 18.40 degrees, 20.90 degrees, 24.00 degrees, and 27.50 degrees;
(h) a crystalline polymorph of irinotecan free base having a powder X-ray diffraction pattern with 20 peaks at 7.10 degrees, 10.60 degrees, 12.40 degrees, 21.60 degrees, and 24.20 degrees;
(i) a crystalline polymorph of irinotecan hydrochloride having a powder X-ray diffraction pattern with 20 peaks at 7.60 degrees, 8.30 degrees, 9.55 degrees, 11.00 degrees, and 12.40 degrees;
(j) a crystalline polymorph of topotecan hydrochloride having a powder X-ray diffraction pattern with 20 peaks at 5.90 degrees, 13.90 degrees, 22.60 degrees, 23.20 degrees, and 26.50 degrees;
(k) a crystalline polymorph of topotecan hydrochloride having a powder X-ray diffraction pattern with 20 peaks at 14.00 degrees, 18.80 degrees, 22.50 degrees, 25.40 degrees, and 25.70 degrees;
(l) a crystalline polymorph of topotecan hydrochloride having a powder X-ray diffraction pattern with 20 peaks at 6.10 degrees, 12.00 degrees, 14.30 degrees, 15.30 degrees, 16.80 degrees, 18.20 degrees, 21.50 degrees, and 23.00 degrees; and
(m) a crystalline polymorph of topotecan hydrochloride having a powder X-ray diffraction pattern with 20 peaks at 5.30 degrees, 11.70 degrees, 13.10 degrees, 15.50 degrees, 16.00 degrees, 16.60 degrees, 17.20 degrees, and 25.40 degrees.

When the improvement is made by use of a stereoisomer, the use can be, but is not limited to, use of a stereoisomeric form of irinotecan, topotecan, or a derivative or analog of irinotecan or topotecan selected from the group consisting of:
(a) specific enantiomers;
(b) racemates; and
(c) preparations enhanced in one specific enantiomer, such as preparations comprising 60%, 65%, 70%, 75%, 80%, 85%, 90%, 95%, 96%, 97%, 98%, or 99% of a specific enantiomer.

Still another aspect of the present invention is a composition to improve the efficacy or reduce the side effects of treatment with irinotecan, topotecan, or a derivative, analog, salt, solvate or prodrug of irinotecan or topotecan wherein the composition comprises:
(a) an alternative selected from the group consisting of:
   (i) a therapeutically effective quantity of irinotecan, topotecan, or a derivative, analog, prodrug, salt, or solvate of irinotecan or topotecan;
   (ii) two or more therapeutically active ingredients comprising:
      (A) a therapeutically effective quantity of irinotecan, topotecan, or a derivative, analog, salt, or solvate of irinotecan or topotecan; and
      (B) at least one additional therapeutic agent, therapeutic agent subject to chemosensitization, therapeutic agent subject to chemopotentiation, or component of a multiple drug system;
   (iii) a therapeutically effective quantity of irinotecan, topotecan, or a derivative, analog, prodrug, salt, or solvate of irinotecan or topotecan that is incorporated into a dosage form;
   (iv) a therapeutically effective quantity of irinotecan, topotecan, or a derivative, analog, prodrug, salt, or solvate of irinotecan or topotecan that is incorporated into a dosage kit and packaging;
   (v) a therapeutically effective quantity of irinotecan, topotecan, or a derivative, analog, prodrug, salt, or solvate of irinotecan or topotecan that is subjected to a bulk drug product improvement;
   (vi) a therapeutically effective quantity of irinotecan, topotecan, or a derivative, analog, prodrug, salt, or solvate of irinotecan or topotecan that is incorporated into a drug delivery system;
   (vii) a therapeutically effective quantity of a prodrug of irinotecan or topotecan or a derivative or analog of irinotecan or topotecan; and
   (viii) a therapeutically effective quantity of irinotecan, topotecan, or a derivative, analog, prodrug, salt, or solvate of irinotecan or topotecan that is incorporated into a liposomal formulation; and
(b) at least one pharmaceutically acceptable diluent, solvent or excipient.

Typically, the irinotecan, topotecan, or a derivative, analog, salt, or solvate of irinotecan or topotecan is irinotecan or topotecan. Suitable derivatives and analogs of irinotecan and topotecan are described above.

Typically, the composition is formulated for treatment of a malignancy. Malignancies treatable by administration of compositions are described above. In another alternative, the composition is formulated for a treatment of a disease or condition selected from the group consisting of: angiogenic diseases; benign prostate hypertrophy; psoriasis; gout; autoimmune conditions; transplantation rejection; restenosis prevention in cardiovascular disease; bone marrow transplantation; infection; AIDS; and Barrett's esophagus.

Suitable additional therapeutic agents, therapeutic agents subject to chemosensitization, or therapeutic agents subject to chemopotentiation are also described above. These additional therapeutic agents, therapeutic agents subject to chemosensitization, and therapeutic agents subject to chemopotentiation, in order to be incorporated in a single pharmaceutical composition with the irinotecan, topotecan, or a derivative, analog, salt, or solvate of irinotecan or topotecan, do not interact negatively with the irinotecan, topotecan, or a derivative, analog, salt, or solvate of irinotecan or topotecan in such a manner that the therapeutic activity or the bioavailability of either agent is significantly reduced.

Suitable dosage forms, dosage kits and packaging, bulk drug product improvements, and drug delivery systems are as stated above.

In compositions according to the present invention, diluents, solvents, or excipients can include, in addition to components described above, components generally described as pharmaceutically acceptable carriers. Such pharmaceutically acceptable carriers can include, but are not limited to, phosphate buffered saline solution, water, emulsions, such as oil/water or water/oil emulsions), and various types of wetting agents, any and all solvents, dispersion media, coatings, sodium lauryl sulfate, isotonic and absorption delaying agents, disintegrants such as potato starch or sodium starch glycolate), and the like, depending on the physical form of the pharmaceutical composition. The carriers also can include stabilizers and preservatives. Still other pharmaceutical excipients and carriers are known in the art, and include, but are not limited to: preservatives; sweetening agents for oral administration; thickening agents; buffers; liquid carriers; wetting, solubilizing, or emulsifying agents; acidifying agents; antioxidants; alkalinizing agents; carrying agents; chelating agents; colorants; complexing agents; suspending or viscosity-increasing agents; flavors or perfumes; oils; penetration enhancers; polymers; stiffening agents; proteins; carbohydrates; bulking agents; and lubricating agents. The use of such agents for pharmaceutically active substances is well known in the art, and suitable agents for inclusion into dosage forms can be chosen according to factors such as the quantity of irinotecan, topotecan, or a derivative, analog, salt, or solvate of irinotecan or topotecan, and, if present, other active agent or agents to be included per unit dose, the intended route of administration, the physical form of the dosage form, and optimization of patient compliance with administration. Except insofar as any conventional medium, carrier, or agent is incompatible with the active ingredient or ingredients, its use in a composition according to the present invention is contemplated.

Pharmaceutical compositions according to the present invention can be formulated for oral, sustained-release oral, buccal, sublingual, inhalation, insufflation, or parenteral administration. Suitable routes for administration of pharmaceutical compositions according to the present invention can be chosen based on factors known to one of skill in the art including the unit dose of the irinotecan, topotecan, or a derivative, analog, salt, or solvate of irinotecan or topotecan, and, if present, the other active agent or agents, the particular carriers or excipients included in the composition, the intended route of administration, the disease or condition to be treated, its severity, other diseases or conditions affecting the and other factors known in the art.

If a pharmaceutical composition according to the present invention is intended for oral administration, it is typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alternative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms. The tablets, pills, troches, capsules, wafers, or other conventional dosage forms can also contain the following: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as coatings or to otherwise modify the physical form and properties of the dosage unit. For instance, tablets, pills, or capsules can be coated with shellac, sugar, or both. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

In one alternative, a sustained-release formulation is used. Sustained-release formulations are well-known in the art. For example, they can include the use of polysaccharides such as xanthan gum and locust bean gum in conjunction with carriers such as dimethylsiloxane, silicic acid, a mixture of mannans and galactans, xanthans, and micronized seaweed, as disclosed in U.S. Pat. No. 6,039,980 to Baichwal. Other sustained-release formulations incorporate a biodegradable polymer, such as the lactic acid-glycolic acid polymer disclosed in U.S. Pat. No. 6,740,634 to Saikawa et al. Still other sustained-release formulations incorporate an expandable lattice that includes a polymer based on polyvinyl alcohol and polyethylene glycol, as disclosed in U.S. Pat. No. 4,428,926 to Keith. Still other sustained-release formulations are based on the Eudragit™ polymers of Rohm & Haas that include copolymers of acrylate and methacrylates with quaternary ammonium groups as functional groups as well as ethylacrylate methylmethacrylate copolymers with a neutral ester group.

Oral liquid preparations can be in the form of, for example, aqueous or oily suspensions, solutions, emulsions, syrups, tinctures, or elixirs, or can be presented as a dry product for reconstitution with water or other suitable vehicles before use. Such liquid preparations can contain conventional additives such as suspending agents, for example, sorbitol syrup, methylcellulose, glucose/sugar syrup, gelatin, hydroxymethylcellulose, carboxymethylcellulose, aluminum stearate gel, or hydrogenated edible fats; emulsifying agents, such as lecithin, sorbitan monooleate, or acacia; non-aqueous vehicles (which may include edible oils), for example, almond oil, fractionated coconut oil, oily esters, propylene glycol, or ethyl alcohol; or preservatives, for example, methylparaben, propylparaben, or sorbic acid. The preparations can also contain buffer salts, flavoring, coloring, or sweetening agents (e.g., mannitol) as appropriate.

When compositions according to the present invention are formulated for parenteral administration, e.g., formulated for injection via the intravenous, intramuscular, subcutaneous, intralesional, or intraperitoneal routes or other routes known in the art, many options are possible. The preparation of an aqueous composition as described above will be known to those of skill in the art. Typically, such compositions can be prepared as injectables, either as liquid solutions and/or suspensions. Solid forms suitable for use to prepare solutions and/or suspensions upon the addition of a liquid prior to injection can also be prepared. The preparations can also be emulsified. The pharmaceutical forms suitable for injectable use include sterile aqueous solutions and/or dispersions; formulations including sesame oil, peanut oil, synthetic fatty acid esters such as ethyl oleate, triglycerides, and/or aqueous propylene glycol; and/or sterile powders for the extemporaneous preparation of sterile injectable solutions and/or dispersions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In all cases the form must be sterile and/or must be fluid to the extent that the solution will pass readily through a syringe and needle of suitable diameter for administration. It must be stable under the conditions of manufacture and storage and must be preserved against the contaminating action of microorganisms, such as bacteria or fungi.

For administration of the irinotecan, topotecan, or a derivative, analog, salt, or solvate of irinotecan or topotecan or of a pharmaceutical composition containing the irinotecan, topotecan, or a derivative, analog, salt, or solvate of irinotecan or topotecan, various factors must be taken into account in setting suitable dosages. These factors include other medications being administered to the subject, which, in some cases, may alter the pharmacokinetics of the irinotecan, topotecan, or a derivative, analog, salt, or solvate of irinotecan or topotecan, either causing it to be degraded more rapidly or more slowly. These medications can, for example, affect either liver or kidney function or may induce the synthesis of one or more cytochrome P450 enzymes that can metabolize the irinotecan, topotecan, or a derivative, analog, salt, or solvate of irinotecan or topotecan.

The exact formulation, route of administration and dosage can be chosen by the individual physician in view of the patient's condition. (See, e.g., Fingl et al., in The Pharmacological Basis of Therapeutics, 1975, ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated, such as, but not limited to, a malignancy, and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

### Advantages of the Invention

The present invention provides improved methods and compositions for treatment of malignancies and other diseases and conditions, including, but not limited to, benign hyperproliferative diseases and conditions, infections, inflammatory diseases and conditions, and immunological diseases and conditions. Irinotecan and topotecan function by inhibiting topoisomerase I, particularly in cancer cells. Methods and compositions according to the present invention are well-tolerated and can be used together with other methods and therapeutic agents for treating malignancy, as well as other diseases.

As used herein in the specification and claims, the transitional phrase “comprising” and equivalent language also encompasses the transitional phrases “consisting essentially of” and “consisting of” with respect to the scope of any claims presented herein, unless the narrower transitional phrases are explicitly excluded. As used herein in the specification and claims, recitation of a method of medical treatment also encompasses use of a compound or composition recited in connection with the method for treatment of the specific diseases or conditions recited in connection with the method.

Methods according to the present invention possess industrial applicability for the preparation of a medicament for the treatment of diseases or conditions described herein, including, but not limited to, malignancy. Methods according to the present invention also possess industrial applicability for use in treating such diseases and conditions, including, but not limited to, malignancy. Compositions according to the present invention possess industrial applicability as pharmaceutical compositions, particularly for the treatment of malignancy, as well as for other diseases and conditions described above.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted to by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. A method to improve the efficacy and/or reduce the side effects of the administration of irinotecan, topotecan, or a derivative or analog of irinotecan or topotecan for treatment of benign or neoplastic hyperproliferative diseases, infections, inflammatory disease or conditions, or immunological diseases or conditions comprising the steps of:
(a) identifying at least one factor or parameter associated with the efficacy and/or occurrence of side effects of the administration of the irinotecan, topotecan, or the derivative or analog of irinotecan or topotecan for the treatment of benign or neoplastic hyperproliferative diseases, infections, inflammatory disease or conditions, or immunological diseases; and
(b) modifying the factor or parameter to improve the efficacy and/or reduce the side effects of the administration of the irinotecan, topotecan, or the derivative or analog of irinotecan or topotecan for the treatment of benign or neoplastic hyperproliferative diseases, infections, inflammatory disease or conditions, or immunological diseases, wherein the factor or parameter is selected from the group consisting of:
(1) dose modification;
(2) route of administration;
(3) schedule of administration;
(4) indications for use;
(5) disease stages;
(6) other indications;
(7) patient selection;
(8) patient or disease phenotype;
(9) patient or disease genotype;
(10) pre-post/treatment preparation;
(11) toxicity management;
(12) pharmacokinetic/pharmacodynamic monitoring;
(13) drug combinations;
(14) chemosensitization;
(15) chemopotentiation;
(16) post-treatment management;
(17) alternative medicine/therapeutic support;
(18) bulk drug product improvements;
(19) diluent systems;
(20) solvent systems;
(21) excipients;
(22) dosage forms;
(23) dosage kits and packaging;
(24) drug delivery systems, wherein the drug delivery system is selected from the group consisting of nanocrystals, liposomes, slow-release injectable gels, microspheres, suspensions with glycerol, meltable drug release suppositories with cocoa butter alone or in combination with PEG, lecithin, or polylactide/polyglycolide, rectal plugs for drug delivery, micro- or nano-emulsions, cyclodextrins, and topical delivery systems;
(25) drug conjugate forms, wherein the drug conjugate form is selected from the group consisting of polyethylene glycols, polylactides, polyglycolides, amino acids, and peptides;
(26) compound analogs;
(27) prodrug systems, wherein the prodrug system is selected from the group consisting of enzyme sensitive esters, dimers, Schiff bases, pyridoxal complexes, caffeine complexes, gastrointestinal system transporters, and permeation enhancers;
(28) multiple drug systems;
(29) biotherapeutic enhancement;
(30) biotherapeutic resistance modulation;
(31) radiation therapy enhancement;
(32) novel mechanisms of action;
(33) selective target cell population therapeutics;
(34) use of liposomes for drug delivery;
(35) use of crystalline polymorphisms; and
(36) use of stereoisomers.

2. (canceled)

3. The method of claim 1 wherein the irinotecan, topotecan, or the derivative or analog of irinotecan or topotecan is selected from the group consisting of: irinotecan; a derivative or analog of irinotecan; topotecan; and a derivative or analog of topotecan.

4.-6. (canceled)

7. The method of claim 1 wherein the method treats a neoplastic hyperproliferative disease.

8. The method of claim 7 wherein the neoplastic hyperproliferative disease is selected from the group consisting of colorectal cancer, pancreatic cancer, lung cancer, breast cancer, gastric cancer, locally advanced or metastatic breast cancer, ovarian cancer, rhabdomyosarcoma, cervical cancer, neuroblastoma, glioblastoma multiforme, Ewing's sarcoma, non-Hodgkin's lymphoma, endometrial cancer, and oligodendroglioma.

9.-10. (canceled)

11. The method of claim 1 wherein the improvement is made by dose modification, wherein the dose modification is selected from the group consisting of:
(a) intravenous infusion for hours to days;
(b) biweekly, tri-weekly, or monthly administration;
(c) doses greater than 100 mg/m 2/day;
(d) progressive escalation of dosing from 100 mg/m 2/day based on patient tolerance;
(e) doses less than 2 mg/m 2 for greater than 14 days;
(f) dose modification associated with use of polyamine to modulate metabolism;
(g) dose modification associated with use of eflornithine to modulate metabolism;
(h) selected and intermittent boost dose administration;
(i) bolus single and multiple doses escalating from 100 mg/m 2;
(j) oral doses below 30 or above 130 mg/m 2;
(k) low potency (1-10 mg/mL) oral solutions or suspensions; and
(l) medium potency (10-200 mg/mL) oral solutions or suspensions.

12.-14. (canceled)

15. The method of claim 1 wherein the improvement is made by schedule of administration, wherein the schedule of administration is selected from the group consisting of:
(a) daily administration;
(b) weekly administration for three weeks;
(c) weekly administration for two weeks;
(d) biweekly administration;
(e) biweekly administration for three weeks with a 1-2 week rest period;
(f) intermittent boost dose administration;
(g) administration daily for one week then once per week for multiple weeks; and
(h) administration daily on days 1-5, 8-12 every three weeks, 2-5 times per day.

16.-22. (canceled)

23. The method of claim 1 wherein the improvement is made by patient selection, wherein the patient selection is selected from the group consisting of:
(a) patients with disease conditions with high levels of metabolic enzymes, histone deacetylase, protein kinases, or ornithine decarboxylase;
(b) patients with disease conditions with low levels of metabolic enzymes, histone deacetylase, protein kinases, or ornithine decarboxylase;
(c) patients with low or high susceptibility to thrombocytopenia or neutropenia;
(d) patients intolerant of GI toxicities;
(e) patients with over- or under-expression of jun, GPCR's and signal transduction proteins, VEGF, prostate specific genes, protein kinases, or telomerases;
(f) patients with high or low levels of activity of UDP-glucuronosyltransferase (UGT);
(g) patients with results of liquid biopsy suggesting variations in treatment;
(h) patients with results of genomic analysis suggesting variations in treatment;
(i) patients with results of proteomic analysis suggesting variations in treatment;
(j) patients with results of BRCA1 or BRCA2 gene analysis suggesting variations in treatment;
(k) patients with wild-type or methylated MGMT promoter;
(l) patients with mutations in IDH1; and
(m) patients with mutations in HER2.

24. (canceled)

25. The method of claim 1 wherein the improvement is made by consideration of patient or disease phenotype, wherein the consideration of patient or disease phenotype is selected from the group consisting of:
(a) diagnostic tools, techniques, kits and assays to confirm a patient's particular phenotype and for the measurement of metabolism-associated enzymes, specific metabolites, level or expression of histone deacetylase, level or expression of protein kinases, ornithine decarboxylase, VEGF, prostate specific genes, protein kinases, telomerase, jun, or GPCR's;
(b) surrogate compound dosing;
(c) detection or analysis of circulating tumor proteins;
(d) low dose drug pre-testing for enzymatic status;
(e) upregulation of protein expression for ERBB2, GRB7, JNK1 kinase, BCL2, MK167, phospho-Akt, CD-68, or BAG1 as associated with responsiveness to treatment of colorectal cancer by irinotecan;
(f) downregulation of protein expression for Erk1 kinase, phospho-GSK-313, MMP11, CTSL2, CCNB1, BIRC5, STK6, MRP14 and GSTM1 as associated with responsiveness to treatment of colorectal cancer by irinotecan;
(g) protein expression for AMD1, CTSC, EIF1AX, C12orf30, DDX54, PTPN2, and TBX3 as affecting therapeutic efficacy of irinotecan;
(h) expression level of topoisomerase I;
(i) activity of carboxylesterase;
(j) activity of ABC transporter genes, including genes for MRP-1, MRP-2, and ABCG2;
(k) plasma level of tissue inhibitor of metalloproteinase-1 (TIMP-1); and
(l) the level of a marker that is one or more of 5-aminoimidazole-4-carboxamide ribotide, alanine, aspartic acid, cysteine, cysteine-glutathione disulfide, glycerol-3-phosphate, histidine, isoleucine, leucine, lysine, methionine sulfoxide, N⁶,N⁶,N⁶-trimethyllysine, N⁶-acetyllysine, octanoic acid, serine, taurocholic acid, threonine, tryptophan, tyrosine, and valine.

26. (canceled)

27. The method of claim 1 where the improvement is made by consideration of patient or disease genotype, wherein the consideration of patient or disease genotype is selected from the group consisting of:
(a) diagnostic tools, techniques, kits and assays to confirm a patient's particular genotype;
(b) gene/protein expression chips and analysis;
(c) single nucleotide polymorphism (SNP) assessment;
(d) SNPs for histone deacetylase, ornithine decarboxylase, S-adenosyl methionine, GPCR's, protein kinases, telomerase, or jun;
(e) identification and measurement of metabolism enzymes and metabolites;
(f) mutation in specific wild-type and mutated genes;
(g) epigenetics via methylation and acetylation;
(h) mutations in genes for UGT, MGMT, BRCA, IDH, He 2, or EGFR;
(i) determination of expression for wild-type or mutated genes;
(j) detection or analysis of circulating tumor DNA or RNA;
(k) use of genome-wide sequencing;
(l) determination of the presence of A or G at genotypic marker −3156 of the UGT1A1 gene or at any position in linkage equilibrium with this genotypic marker wherein A positively correlates with irinotecan toxicity and G correlates with the absence of irinotecan toxicity, such that homozygosity for A indicates increased toxicity;
(m) a genotypic marker associated with polymorphisms in the TATA box within the promoter region for the UGT1A1 gene such that the presence of 7 TA repeats in the TATA box reduces expression of UGT1A1 and predisposes to increased toxicity;
(n) occurrence of variant alleles of MRP1;
(o) existence of single nucleotide polymorphisms in a region encoding APCDD1L, R3HCC1, OR5112, MKKS, EDEM3, or ACOX1;
(p) a polymorphism that is (G/G) for rs1792689, (C/T) or (C/C) for rs2268753; (G/G) for rs17776182, (NA) for rs7570532, or (NG) or (G/G) for rs4946935 which is favorable for efficacy of irinotecan when administered together with bevacizumab;
(q) a polymorphism that is (G/G) for rs1792689, (C/T) or (C/C) for rs2268753, (G/G) for rs17776182, (A/A) for rs7570532, and (A/G) or (G/G) for rs4946935, which is unfavorable for efficacy of irinotecan when administered together with bevacizumab; and
(r) the occurrence of a polymorphism rs1980576 in APCDD1L which is A in the wild-type and G in the mutant and where irinotecan has the strongest therapeutic effect when the genome is homozygous for A.

28.-34. (canceled)

35. The method of claim 1 wherein the improvement is made by use of a drug combination, wherein the drug combination is selected from the group consisting of:
(a) use with other topoisomerase inhibitors;
(b) use with fraudulent nucleosides;
(c) use with fraudulent nucleotides;
(d) use with thymidylate synthetase inhibitors;
(e) use with signal transduction inhibitors;
(f) use with cisplatin or platinum-containing analogs;
(g) use with alkylating agents selected from the group consisting of BCNU, Gliadel wafers, CCNU, bendamustine (Treanda), and temozolomide (Temodar);
(h) use with anti-tubulin agents;
(i) use with antimetabolites;
(j) use with berberine;
(k) use with apigenin;
(l) use with amonafide;
(m) use with colchicine or colchicine analogs;
(n) use with genistein;
(o) use with cytarabine;
(p) use with vinca alkaloids;
(q) use with 5-fluorouracil;
(r) use with curcumin;
(s) use with NF-κB inhibitors;
(t) use with rosmarinic acid;
(u) use with dibromodulcitol;
(v) use with biological therapies selected from the group consisting of Avastin, Rituxan, Herceptin, Erbitux, PD-1 and PDL-1 inhibitors;
(w) use with prednimustine;
(x) use with DNA or RNA therapeutics;
(y) use with Braf inhibitors;
(z) use with BTK inhibitors;
(aa) use with 5-azacytidine;
(ab) use with decitabine;
(ac) use with PARP inhibitors;
(ad) use with hypomethylating agents;
(ae) use with histone deacetylase inhibitors;
(af) use with thalidomide;
(ag) use with trifluridine;
(ah) use with tipiracil hydrochloride;
(ai) use with aflibercept;
(aj) use with 5-(5-(2-(3-aminopropoxy)-6-methoxyphenyl)-1H-pyrazol-3-ylamino)pyrazine-2-carbonitrile;
(ak) use with EGFR inhibitors;
(al) use with VEGF inhibitors;
(am) use with a humanized anti-EGFR IgG1 antibody;
(an) use with 4-iodo-3-nitrobenzamide or metabolites thereof;
(ao) use with immunotherapies selected from the group consisting of: antibodies binding to alpha-PDL1, alpha-44BB, alpha-CTLA4, or alpha-OX40; atezolizumab, avelimumab, nivolumab, pembrolizumab, ipilimumab, tremelimumab, and durvalumab; prexasertib; aldozurubicin; lurbinectedin; and Notch ADC-modulating agents such as rovalpituzumab tesirine; and dilpacimab; and
(ap) use with an MRP inhibitor selected from the group consisting of: valspodar (SDZ-PSC 833), tert-butyl 2-[(3S,6S,9S,15S,21S,24S,27S,30S)-15,18-bis[(2S)-butan-2-yl]-6-[(4-methoxyphenyl)methyl]-3,10,16,19,22,28-hexamethyl-2,5,8,11,14,17,20,23,26,29-decaoxo-9,24,27-tri(propan-2-yl)-4-oxa-1,7,10,13,16,19,22,25,28-nonazabicyclo[28.4.0]tetratriacontan-21-yl]acetate (SDZ 280-446), sodium 3-[[3-[(E)-2-(7-chloroquinolin-2-yl)ethenyl]phenyl]-[3-(dimethylamino)-3-oxopropyl]sulfanylmethyl]sulfanylpropanoate (MK571), dofequidar (MS209), 2-(4-benzhydrylpiperazin-1-yl)ethyl 5-[(4R,6R)-4,6-dimethyl-2-oxo-1,3,2λ-5-dioxaphosphinan-2-yl]-2,6-dimethyl-4-(3-nitrophenyl)pyridine-3-carboxylate (PAK-104p), verapamil, benzbromarone, dipyridamole, furosemide, gamma-GS(naphthyl)cysteinyl-glycine diethyl ester, genistein, quinidine, rifampicin, mifepristone (RU-486), and sulfinpyrazone.

36.-56. (canceled)

57. The method of claim 1 wherein the improvement is made by drug delivery systems, wherein the drug delivery system is selected from the group consisting of:
(a) nanocrystals;
(b) liposomes;
(c) slow-release injectable gels;
(d) microspheres;
(e) suspensions with glycerol;
(f) meltable drug release suppositories with cocoa butter alone or in combination with PEG, lecithin, or polylactide/polyglycolide;
(g) rectal plugs for drug delivery;
(h) micro- or nano-emulsions;
(i) cyclodextrins; and
(j) topical delivery systems.

58. (canceled)

59. The method of claim 1 wherein the improvement is made by drug conjugate forms, wherein the drug conjugate form is selected from the group consisting of:
(a) polyethylene glycols;
(b) polylactides;
(c) polyglycolides;
(d) amino acids; and
(e) peptides.

60.-62. (canceled)

63. The method of claim 1 wherein the improvement is made by prodrug systems, wherein the prodrug system is selected from the group consisting of:
(a) enzyme sensitive esters;
(b) dimers;
(c) Schiff bases;
(d) pyridoxal complexes;
(e) caffeine complexes;
(f) gastrointestinal system transporters; and
(g) permeation enhancers.

64.-76. (canceled)

77. The method of claim 1 wherein the improvement is made by use of liposomes for drug delivery, wherein the liposome is a liposomal formulation for the delivery of irinotecan, topotecan, or a derivative or analog thereof selected from the group consisting of:
(a) a liposomal formulation comprising a first liposome-forming material comprising cardiolipin and a second liposome-forming material, wherein the composition comprises from about 1 weight percent to about 50 weight percent irinotecan, about 1 weight percent to about 95 weight percent of phosphatidylcholine, and about 0.001 to about 5 weight percent of α-tocopherol for the delivery of irinotecan;
(b) a liposomal formulation wherein the liposome comprises a liposome formed by a membrane of a lipid bilayer containing a phospholipid as a membrane component, wherein only the outer surface of the liposome is modified with a surface-modifying agent containing a polyethylene glycol, in which irinotecan and/or a salt thereof is encapsulated at a concentration of at least 0.1 mol/mol (drug mol/membrane total lipid mol) by an ion gradient between an inner aqueous phase and an outer aqueous phase of the liposome for the delivery of irinotecan;
(c) a liposome comprising irinotecan or irinotecan hydrochloride, neutral phospholipid, and cholesterol, wherein the weight ratio of the cholesterol to the neutral phospholipid is about 1:3 to about 1:5, and in which the liposome can comprise irinotecan hydrochloride, hydrogenated soybean phosphatidylcholine, polyethylene glycol 2000-distearoyl phosphatidyl ethanolamine, cholesterol, and ethylenediaminetetraacetic acid disodium, wherein the weight ratio of the cholesterol to the hydrogenated soybean phosphatidylcholine is about 1:4 for the delivery of irinotecan;
(d) a liposomal formulation comprising irinotecan sucrose octasulfate 1,2-distearoyl-sn-glycero-3-phosphocholine (DSPC), cholesterol, and a N-(carbonylmethoxypolyethylene glycol-2000)-1,2-distearoyl-sn-glycero-3-phosphoethanolamine for the delivery of irinotecan;
(e) a liposomal formulation wherein the interior of the liposome includes a substituted ammonium moiety of Formula (AM-I): 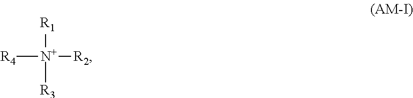
wherein each of R₁, R₂, R₃, and R₄ is independently a hydrogen or an organic group having, inclusively, in totality up to 18 carbon atoms, wherein at least one of R₁, R₂, R₃, and R₄ is an organic group, wherein the organic group is independently a hydrocarbon group having up to 8 carbon atoms, and is an alkyl, alkylidene, heterocyclic alkyl, cycloalkyl, aryl, alkenyl, or cycloalkenyl group or a hydroxy-substituted derivative thereof, optionally including within its hydrocarbon chain a S, O, or N atom, forming an ether, ester, thioether, amine, or amide bond, wherein at least three of R₁, R₂, R₃, and R₄ are organic groups, or the substituted ammonium is a sterically hindered ammonium, such as, for example, where at least one of the organic groups has a secondary or tertiary carbon atom directly linked to the ammonium nitrogen atom for the delivery of irinotecan;
(f) a liposomal formulation wherein the inner space of the liposome contains a polyanion and wherein the polyanion is a polyanionized polyol or a polyanionized sugar, in which suitable substituted ammonium compounds include isopropylethylammonium, isopropylmethylammonium, diisopropylammonium, t-butylethylammonium, dicychohexylammonium, protonized forms of morpholine, pyridine, piperidine, pyrrolidine, piperazine, t-butylamine, 2-amino-2-methylpropanol-1,2-amino-2-methyl-propandiol-1,3, tris-(hydroxyethyl)-aminomethane, trimethylammonium, triethylammonium, tributyl ammonium, diethylmethylammonium, diisopropylethyl ammonium, triisopropylammonium, N-methylmorpholinium, N-hydroxyethylpiperidinium, N-methylpyrrolidinium, N,N′-dimethylpiperazinium, tetramethylammonium, tetraethylammonium, and tetrabutylammonium, and in which the membrane of the liposome can constitute a polymer-conjugated ligand for delivery of irinotecan;
(g) a liposomal formulation wherein the liposome comprises cardiolipin and a second liposome-forming material that is a lipid selected from the group consisting of phosphatidylcholine, cholesterol, α-tocopherol, dipalmitoyl phosphatidylcholine and phosphatidylserine for delivery of irinotecan;
(h) a liposomal formulation wherein the lipid phase comprises cardiolipin and at least one additional lipid component selected from the group consisting of phosphatidylcholine, phosphatidylethanolamine, phosphatidylserine, phosphatidylglycerol, phosphatidic acid, phosphatidylinositol, sphingomyelin, sterol, tocopherol, fatty acid, and mixtures thereof for delivery of irinotecan;
(i) a liposomal formulation wherein the liposomal composition comprises comprising irinotecan sucrose octasulfate (SOS) encapsulated in liposomes comprising one or more phospholipids with a ratio corresponding to a total of 500 grams irinotecan moiety (±10% by weight) per mol total phospholipids, the liposomal irinotecan composition stabilized to have less than 20 mol % (with respect to total phospholipids) lysophosphatidylcholine during the first 6 months of storage of the liposomal irinotecan composition at about 4° C. for delivery of irinotecan; and
(j) a liposomal formulation suspension having selected liposome sizes in the size range between 0.05 and 0.25 lam, and between about 85%-100% liposome-entrapped topotecan, wherein the liposomes can further comprise a cryoprotectant such as sucrose, trehalose, lactose, maltose, cyclodextrin, polyethylene glycol, dextran, polyvinylpyrrolidone, and hydroxyethyl starch, and can comprise lipids such as cholesterol, phosphatidylcholines, sphingomyelins, phosphatidylglycerols, phosphatidic acids, phosphatidylethanolamines, phosphatidylinositols, phosphatidylserines, cholesterol sulfate, or cholesterol hemisuccinate; wherein the lipid used may be conjugated to a hydrophilic polymer such as polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and polyglycerol for the delivery of topotecan.

78. (canceled)

79. The method of claim 1 wherein the improvement is made by crystalline polymorphs, wherein the crystalline polymorph is a crystalline polymorph of irinotecan, topotecan, or a derivative or analog of irinotecan or topotecan selected from the group consisting of:
(a) a crystalline polymorph of irinotecan hydrochloride having a powder X-ray diffraction pattern with 20 peaks at 20.3956 degrees, 22.2950 degrees, 12.0744 degrees, 8.4800 degrees, and 11.8306 degrees;
(b) a crystalline polymorph of irinotecan hydrochloride having a powder X-ray diffraction pattern with 20 peaks at 23.9600 degrees, 20.9200 degrees, and 21.0800 degrees;
(c) a crystalline polymorph of irinotecan hydrochloride having a powder X-ray diffraction pattern with 20 peaks at 12.3406 degrees, 24.7913 degrees, 10.9438 degrees, 8.2056 degrees, 27.6750 degrees, 22.7206 degrees, and 21.2350 degrees;
(d) a crystalline polymorph of irinotecan hydrochloride having a powder X-ray diffraction pattern with 20 peaks at 9.1912 degrees, 9.9800 degrees, 18.8937 degrees, 15.2725 degrees, 16.1681 degrees, 25.7400 degrees, and 27.0662 degrees;
(e) a crystalline polymorph of irinotecan hydrochloride having a powder X-ray diffraction pattern with 20 peaks at 9.15 degrees, 10.00 degrees, 11.80 degrees, 12.20 degrees, 13.00 degrees, and 13.40 degrees;
(f) a crystalline polymorph of irinotecan hydrochloride having a powder X-ray diffraction pattern with 20 peaks at 8.5300 degrees, 9.0400 degrees, 10.23 degrees, 11.65 degrees, 17.01 degrees, 18.08 degrees, 19.17 degrees, and 24.30 degrees;
(g) a crystalline polymorph of irinotecan free base having a powder X-ray diffraction pattern with 20 peaks at 8.70 degrees, 13.10 degrees, 14.50 degrees, 17.40 degrees, 18.40 degrees, 20.90 degrees, 24.00 degrees, and 27.50 degrees;
(h) a crystalline polymorph of irinotecan free base having a powder X-ray diffraction pattern with 20 peaks at 7.10 degrees, 10.60 degrees, 12.40 degrees, 21.60 degrees, and 24.20 degrees;
(i) a crystalline polymorph of irinotecan hydrochloride having a powder X-ray diffraction pattern with 20 peaks at 7.60 degrees, 8.30 degrees, 9.55 degrees, 11.00 degrees, and 12.40 degrees;
(j) a crystalline polymorph of topotecan hydrochloride having a powder X-ray diffraction pattern with 20 peaks at 5.90 degrees, 13.90 degrees, 22.60 degrees, 23.20 degrees, and 26.50 degrees;
(k) a crystalline polymorph of topotecan hydrochloride having a powder X-ray diffraction pattern with 20 peaks at 14.00 degrees, 18.80 degrees, 22.50 degrees, 25.40 degrees, and 25.70 degrees;
(l) a crystalline polymorph of topotecan hydrochloride having a powder X-ray diffraction pattern with 20 peaks at 6.10 degrees, 12.00 degrees, 14.30 degrees, 15.30 degrees, 16.80 degrees, 18.20 degrees, 21.50 degrees, and 23.00 degrees; and
(m) a crystalline polymorph of topotecan hydrochloride having a powder X-ray diffraction pattern with 20 peaks at 5.30 degrees, 11.70 degrees, 13.10 degrees, 15.50 degrees, 16.00 degrees, 16.60 degrees, 17.20 degrees, and 25.40 degrees.

80. (canceled)

81. The method of claim 1 wherein the improvement is made by use of a stereoisomer, wherein the stereoisomer is a stereoisomeric form of irinotecan, topotecan, or a derivative or analog of irinotecan or topotecan selected from the group consisting of:
(a) specific enantiomers;
(b) racemates; and
(c) preparations enhanced in one specific enantiomer comprising 60%, 65%, 70%, 75%, 80%, 85%, 90%, 95%, 96%, 97%, 98%, or 99% of a specific enantiomer.

82. (canceled)

83. A composition to improve the efficacy or reduce the side effects of treatment with irinotecan, topotecan, or a derivative, analog, prodrug, salt, solvate or prodrug of irinotecan or topotecan wherein the composition comprises:
(a) an alternative selected from the group consisting of:
(i) a therapeutically effective quantity of irinotecan, topotecan, or a derivative, analog, prodrug, salt, or solvate of irinotecan or topotecan;
(ii) two or more therapeutically active ingredients comprising:
(A) a therapeutically effective quantity of irinotecan, topotecan, or a derivative, analog, prodrug, salt, or solvate of irinotecan or topotecan; and
(B) at least one additional therapeutic agent, therapeutic agent subject to chemosensitization, therapeutic agent subject to chemopotentiation, or component of a multiple drug system;
(iii) a therapeutically effective quantity of irinotecan, topotecan, or a derivative, analog, prodrug, salt, or solvate of irinotecan or topotecan that is incorporated into a dosage form;
(iv) a therapeutically effective quantity of irinotecan, topotecan, or a derivative, analog, prodrug, salt, or solvate of irinotecan or topotecan that is incorporated into a dosage kit and packaging;
(v) a therapeutically effective quantity of irinotecan, topotecan, or a derivative, analog, prodrug, salt, or solvate of irinotecan or topotecan that is subjected to a bulk drug product improvement;
(vi) a therapeutically effective quantity of irinotecan, topotecan, or a derivative, analog, prodrug, salt, or solvate of irinotecan or topotecan that is incorporated into a drug delivery system, wherein the drug delivery system is selected from the group consisting of nanocrystals, liposomes, slow-release injectable gels, microspheres, suspensions with glycerol, meltable drug release suppositories with cocoa butter alone or in combination with PEG, lecithin, or polylactide/polyglycolide, rectal plugs for drug delivery, micro- or nano-emulsions, cyclodextrins, and topical delivery systems;
(vii) a therapeutically effective quantity of irinotecan, topotecan, or a derivative, analog, prodrug, salt, or solvate of irinotecan or topotecan that is in a drug conjugate form, wherein the drug conjugate form is selected from the group consisting of polyethylene glycols, polylactides, polyglycolides, amino acids, and peptides; and
(viii) a therapeutically effective quantity of a prodrug of irinotecan or topotecan or a derivative or analog of irinotecan or topotecan, wherein the prodrug is selected from the group consisting of enzyme sensitive esters, dimers, Schiff bases, pyridoxal complexes, caffeine complexes, gastrointestinal system transporters, and permeation enhancers; and
(b) at least one pharmaceutically acceptable diluent, solvent or excipient.

84. The composition of claim 83 wherein the irinotecan, topotecan, or the derivative or analog of irinotecan or topotecan is selected from the group consisting of: irinotecan; a derivative or analog of irinotecan; topotecan; and a derivative or analog of topotecan.

85.-87. (canceled)

88. The composition of claim 83 wherein the composition is formulated to treat a neoplastic hyperproliferative disease.

89.-91. (canceled)

92. The composition of claim 83 wherein the composition comprises two or more active ingredients comprising:
(a) a therapeutically effective quantity of irinotecan, topotecan, or a derivative, analog, prodrug, salt, or solvate of irinotecan or topotecan; and
(b) at least one additional therapeutic agent, wherein the at least one additional therapeutic agent is selected from the group consisting of:
(i) other topoisomerase inhibitors;
(ii) fraudulent nucleosides;
(iii) fraudulent nucleotides;
(iv) thymidylate synthetase inhibitors;
(v) signal transduction inhibitors;
(vi) cisplatin or platinum-containing analogs;
(vii) alkylating agents selected from the group consisting of BCNU, Gliadel wafers, CCNU, bendamustine (Treanda), and temozolomide (Temodar);
(viii) anti-tubulin agents;
(ix) antimetabolites;
(X) berberine;
(xi) apigenin;
(xii) amonafide;
(xiii) colchicine or colchicine analogs;
(xiv) genistein;
(xv) cytarabine;
(xvi) vinca alkaloids;
(xvii) 5-fluorouracil;
(xviii) curcumin;
(xix) NF-κB inhibitors;
(xx) rosmarinic acid;
(xxi) dibromodulcitol;
(xxii) biological therapies selected from the group consisting of Avastin, Rituxan, Herceptin, Erbitux, PD-1 and PDL-1 inhibitors;
(xxiii) prednimustine;
(xxiv) DNA or RNA therapeutics;
(xxv) Braf inhibitors;
(xxvi) BTK inhibitors;
(xxvii) 5-azacytidine;
(xxviii) decitabine;
(xxix) PARP inhibitors;
(xxx) hypomethylating agents;
(xxxi) histone deacetylase inhibitors;
(xxxii) thalidomide;
(xxxiii) trifluridine;
(xxxiv) tipiracil hydrochloride;
(xxxv) with aflibercept;
(xxxvi) 5-(5-(2-(3-aminopropoxy)-6-methoxyphenyl)-1H-pyrazol-3-ylamino)pyrazine-2-carbonitrile;
(xxxvii) EGFR inhibitors;
(xxxviii) use with VEGF inhibitors;
(xxxix) a humanized anti-EGFR IgG1 antibody;
(xl) 4-iodo-3-nitrobenzamide or metabolites thereof;
(xli) immunotherapies selected from the group consisting of: antibodies binding to alpha-PDL1, alpha-44BB, alpha-CTLA4, or alpha-OX40; atezolizumab, avelimumab, nivolumab, pembrolizumab, ipilimumab, tremelimumab, and durvalumab; prexasertib; aldozurubicin; lurbinectedin; and Notch ADC-modulating agents such as rovalpituzumab tesirine; and dilpacimab; and
(xlii) an MRP inhibitor selected from the group consisting of: valspodar (SDZ-PSC 833), tert-butyl 2-[(3S,6S,9S,15S,21S,24S,27S,30S)-15,18-bis[(2S)-butan-2-yl]-6-[(4-methoxyphenyl)methyl]-3,10,16,19,22,28-hexamethyl-2,5,8,11,14,17,20,23,26,29-decaoxo-9,24,27-tri(propan-2-yl)-4-oxa-1,7,10,13,16,19,22,25,28-nonazabicyclo[28.4.0]tetratriacontan-21-yl]acetate (SDZ 280-446), sodium 3-[[3-[(E)-2-(7-chloroquinolin-2-yl)ethenyl]phenyl]-[3-(dimethylamino)-3-oxopropyl]sulfanylmethyl]sulfanylpropanoate (MK571), dofequidar (MS209), 2-(4-benzhydrylpiperazin-1-yl)ethyl 5-[(4R,6R)-4,6-dimethyl-2-oxo-1,3,2λ-5-dioxaphosphinan-2-yl]-2,6-dimethyl-4-(3-nitrophenyl)pyridine-3-carboxylate (PAK-104p), verapamil, benzbromarone, dipyridamole, furosemide, gamma-GS(naphthyl)cysteinyl-glycine diethyl ester, genistein, quinidine, rifampicin, mifepristone (RU-486), and sulfinpyrazone.

93.-103. (canceled)

104. The composition of claim 83 wherein the therapeutically effective quantity of irinotecan, topotecan, or a derivative, analog, prodrug, salt, or solvate of irinotecan or topotecan is incorporated into a drug delivery system, wherein the drug delivery system is selected from the group consisting of:
(i) nanocrystals;
(ii) liposomes;
(iii) slow-release injectable gels;
(iv) microspheres;
(v) suspensions with glycerin;
(vi) meltable drug release suppositories with cocoa butter alone or in combination with PEG, lecithin, or polylactide/polyglycolide;
(vii) rectal plugs for drug delivery;
(viii) micro- or nano-emulsions;
(ix) cyclodextrins; and
(x) topical delivery systems.

105. (canceled)

106. The composition of claim 83 wherein the composition comprises a therapeutically effective quantity of a prodrug of irinotecan or topotecan or a derivative or analog of irinotecan or topotecan, wherein the prodrug comprises:
(i) enzyme sensitive esters;
(ii) dimers;
(iii) Schiff bases;
(iv) pyridoxal complexes;
(v) caffeine complexes;
(vi) gastrointestinal system transporters; and
(vii) permeation enhancers.

107. (canceled)

108. The composition of claim 83 wherein the composition comprises a therapeutically effective quantity of irinotecan, topotecan, or a derivative, analog, prodrug, salt, or solvate of irinotecan or topotecan that is in a drug conjugate form, wherein the drug conjugate form is selected from the group consisting of:
(i) polyethylene glycols;
(ii) polylactides;
(iii) polyglycolides;
(iv) amino acids; and
(v) peptides.

109. (canceled)

110. The composition of claim 83 wherein the therapeutically effective quantity of irinotecan, topotecan, or a derivative, analog, prodrug, salt, or solvate of irinotecan or topotecan that is incorporated into a liposomal formulation, wherein the liposomal formulation is selected from the group consisting of:
(i) a liposomal formulation comprising a first liposome-forming material comprising cardiolipin and a second liposome-forming material, wherein the composition comprises from about 1 weight percent to about 50 weight percent irinotecan, about 1 weight percent to about 95 weight percent of phosphatidylcholine, and about 0.001 to about 5 weight percent of α-tocopherol for the delivery of irinotecan;
(ii) a liposomal formulation wherein the liposome comprises a liposome formed by a membrane of a lipid bilayer containing a phospholipid as a membrane component, wherein only the outer surface of the liposome is modified with a surface-modifying agent containing a polyethylene glycol, in which irinotecan and/or a salt thereof is encapsulated at a concentration of at least 0.1 mol/mol (drug mol/membrane total lipid mol) by an ion gradient between an inner aqueous phase and an outer aqueous phase of the liposome for the delivery of irinotecan;
(iii) a liposome comprising irinotecan or irinotecan hydrochloride, neutral phospholipid, and cholesterol, wherein the weight ratio of the cholesterol to the neutral phospholipid is about 1:3 to about 1:5, and in which the liposome can comprise irinotecan hydrochloride, hydrogenated soybean phosphatidylcholine, polyethylene glycol 2000-distearoyl phosphatidyl ethanolamine, cholesterol, and ethylenediaminetetraacetic acid disodium, wherein the weight ratio of the cholesterol to the hydrogenated soybean phosphatidylcholine is about 1:4 for the delivery of irinotecan;
(iv) a liposomal formulation comprising irinotecan sucrose octasulfate 1,2-distearoyl-sn-glycero-3-phosphocholine (DSPC), cholesterol, and a N-(carbonylmethoxypolyethylene glycol-2000)-1,2-distearoyl-sn-glycero-3-phosphoethanolamine for the delivery of irinotecan;
(v) a liposomal formulation wherein the interior of the liposome includes a substituted ammonium moiety of Formula (AM-I): 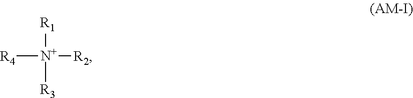
wherein each of R₁, R₂, R₃, and R₄ is independently a hydrogen or an organic group having, inclusively, in totality up to 18 carbon atoms, wherein at least one of R₁, R₂, R₃, and R₄ is an organic group, wherein the organic group is independently a hydrocarbon group having up to 8 carbon atoms, and is an alkyl, alkylidene, heterocyclic alkyl, cycloalkyl, aryl, alkenyl, or cycloalkenyl group or a hydroxy-substituted derivative thereof, optionally including within its hydrocarbon chain a S, O, or N atom, forming an ether, ester, thioether, amine, or amide bond, wherein at least three of R₁, R₂, R₃, and R₄ are organic groups, or the substituted ammonium is a sterically hindered ammonium, such as, for example, where at least one of the organic groups has a secondary or tertiary carbon atom directly linked to the ammonium nitrogen atom for the delivery of irinotecan;
(vi) a liposomal formulation wherein the inner space of the liposome contains a polyanion and wherein the polyanion is a polyanionized polyol or a polyanionized sugar, in which suitable substituted ammonium compounds include isopropylethylammonium, isopropylmethylammonium, diisopropylammonium, t-butylethylammonium, dicychohexylammonium, protonized forms of morpholine, pyridine, piperidine, pyrrolidine, piperazine, t-butylamine, 2-amino-2-methylpropanol-1,2-amino-2-methyl-propandiol-1,3, tris-(hydroxyethyl)-aminomethane, trimethylammonium, triethylammonium, tributyl ammonium, diethylmethylammonium, diisopropylethyl ammonium, triisopropylammonium, N-methylmorpholinium, N-hydroxyethylpiperidinium, N-methylpyrrolidinium, N,N′-dimethylpiperazinium, tetramethylammonium, tetraethylammonium, and tetrabutylammonium, and in which the membrane of the liposome can constitute a polymer-conjugated ligand for delivery of irinotecan;
(vii) a liposomal formulation wherein the liposome comprises cardiolipin and a second liposome-forming material that is a lipid selected from the group consisting of phosphatidylcholine, cholesterol, α-tocopherol, dipalmitoyl phosphatidylcholine and phosphatidylserine for delivery of irinotecan;
(viii) a liposomal formulation wherein the lipid phase comprises cardiolipin and at least one additional lipid component selected from the group consisting of phosphatidylcholine, phosphatidylethanolamine, phosphatidylserine, phosphatidylglycerol, phosphatidic acid, phosphatidylinositol, sphingomyelin, sterol, tocopherol, fatty acid, and mixtures thereof for delivery of irinotecan;
(ix) a liposomal formulation wherein the liposomal composition comprises comprising irinotecan sucrose octasulfate (SOS) encapsulated in liposomes comprising one or more phospholipids with a ratio corresponding to a total of 500 grams irinotecan moiety (±10% by weight) per mol total phospholipids, the liposomal irinotecan composition stabilized to have less than 20 mol % (with respect to total phospholipids) lysophosphatidylcholine during the first 6 months of storage of the liposomal irinotecan composition at about 4° C. for delivery of irinotecan; and
(x) a liposomal formulation suspension having selected liposome sizes in the size range between 0.05 and 0.25 lam, and between about 85%-100% liposome-entrapped topotecan, wherein the liposomes can further comprise a cryoprotectant such as sucrose, trehalose, lactose, maltose, cyclodextrin, polyethylene glycol, dextran, polyvinylpyrrolidone, and hydroxyethyl starch, and can comprise lipids such as cholesterol, phosphatidylcholines, sphingomyelins, phosphatidylglycerols, phosphatidic acids, phosphatidylethanolamines, phosphatidylinositols, phosphatidylserines, cholesterol sulfate, or cholesterol hemisuccinate; wherein the lipid used may be conjugated to a hydrophilic polymer such as polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and polyglycerol for the delivery of topotecan.

111.-112. (canceled)

113. The composition of claim 83 wherein the composition is formulated for oral, sustained-release oral, buccal, sublingual, inhalation, insufflation, or parenteral administration.

114-115. (canceled)
